(19) United States
(12) Patent Application Publication
(10) Pub. No.: US 2020/0071748 A1
(43) Pub. Date: Mar. 5, 2020

(54) METHOD OF DIAGNOSING HEART DISEASE THROUGH BACTERIAL METAGENOME ANALYSIS

(71) Applicant: MD HEALTHCARE INC., Seoul (KR)

(72) Inventor: Yoon-Keun Kim, Gyeonggi-do (KR)

(21) Appl. No.: 16/469,193

(22) PCT Filed: Dec. 15, 2017
(86) PCT No.: PCT/KR2017/014815

§ 371 Date: Jun. 13, 2019

(30) Foreign Application Priority Data
Dec. 16, 2016 (KR) 10-2016-0172414
Dec. 14, 2017 (KR) 10-2017-0172461

(51) Int. Cl. C12Q 1/689; C12Q 1/6883

(52) U.S. Cl. CPC C12Q 1/689; C12Q 2600/112; C12Q 2600/118; C12Q 1/6883

(57) ABSTRACT

The present invention relates to a method of diagnosing heart disease through bacterial metagenome analysis. More specifically, the present invention relates to a method of predicting the causative factor and onset of heart disease, diagnosing heart disease early, and predicting prognosis by analyzing the increase or decrease in the content of an extracellular vesicle derived from specific bacteria by performing bacterial metagenome analysis using a subject-derived sample. Through metagenome analysis of a bacteria-derived extracellular vesicle using a human body-derived sample according to the present invention, the risk of the onset of heart disease is predicted in advance, and thus a risk group for heart disease is diagnosed and predicted early. Accordingly, through suitable management, the moment of onset of disease can be performed even after the onset of disease. Therefore, the rate of onset of heart disease can be reduced, and the effect of treatment can be improved. In addition, since exposure to causative factors is avoided through metagenome analysis in a patient diagnosed with heart disease, the prognosis of heart disease can be improved, or reoccurrence of heart disease can be prevented.

### TECHNICAL FIELD

The present disclosure relates to a method of diagnosing a heart disease through bacterial metagenomic analysis, and more particularly, to a method of diagnosing a risk factor for a heart disease by analyzing an increase or decrease in content of extracellular vesicles derived from specific bacteria by bacterial metagenomic analysis using a sample derived from a subject.

### BACKGROUND ART

Heart disease is a disease occurring in the heart, and examples of major diseases thereof include ischemic heart disease, coronary artery disease, angina, myocardial infarction, arrhythmia, and the like. Among these, coronary artery disease is a group of diseases including angina, myocardial infarction, and the like and is also known as ischemic heart disease. Myocardial infarction is a disease in which an infarction occurs in heart muscles due to blocking of blood vessels that is caused by thrombosis in the coronary arteries. As risk factors for coronary artery disease, hypertension, smoking, diabetes, lack of exercise, obesity, hyperlipidemia, excessive drinking, and the like are known. It has been reported that the above-described disease is prevented when fruit- and vegetable-rich foods are consumed, and a risk for the disease is increased when trans fat-rich foods are consumed.

Dilated cardiomyopathy, which is a disease in which the heart is expanded and unable to contract properly, is known as the most common cause of non-coronary artery disease and cardiomyopathy. Toxins, metabolic disorders, infectious factors, and the like have been suggested as causative factors, but causative factors have not yet been found.

Angina is a disease occurring due to narrowing of the coronary arteries, through which blood is supplied to the heart, causing the volume of blood flowing to heart muscles to fall short of the required amount. When the coronary arteries are narrowed, blood supply is insufficient when in need thereof, and thus chest pain symptoms occur, and when angina remains untreated, it develops into acute myocardial infarction, thus causing sudden death. Angina may be broadly divided into stable angina, unstable angina, and variant angina. Among these three types, while stable angina and unstable angina occur mostly by atherosclerotic plaques, variant angina occurs due to a different cause, which thus has its name. Unlike general angina occurring due to narrowing of blood vessels, variant angina is a disease occurring due to the occurrence of spasms and poor blood circulation although the diameter of blood vessels of the coronary arteries is normal. Variant angina causes chest pain due to a decrease in or blocking of blood flow by spasm-derived contractions, and these symptoms occur early the next morning when drinking or receiving a lot of stress, or the like.

Atrial fibrillation is a condition in which the atria beat continuously and irregularly and a rapid and irregular heart rate occurs. The atria beat 300 to 400 times per minute, while most stimuli are blocked by the atrioventricular (AV) node, and stimuli delivered to the ventricles are approximately 75 to 175 per minute. Causes of the disease include fundamental heart problems, i.e., coronary artery disease, myocardial infarction, hypertension, and mitral valve, and further include pericarditis, pulmonary embolisms, hyperthyroidism or hypothyroidism, septicaemia, diabetes, excessive drinking, pheochromocytoma, and the like.

According to data released by the Korea National Statistical Office in 2013, the three leading causes of death in Korea are malignant neoplasm (cancer) accounting for 28.3%, cerebrovascular disease accounting for 9.6%, and heart disease accounting for 9.5%, and these account for about 50% of all causes of death. It has also been reported that coronary artery disease and cardiomyopathy account for 50% or greater of the causes of death due to heart disease. Modern people face stress caused by conflicts such as economic problems, workplace problems, and the like, and thus blood pressure, blood sugar, and blood fat are increased, and coronary artery disease and cardiomyopathy are very closely associated with diseases such as obesity, smoking, atherosclerosis, and the like, which continue to increase in modern people, and therefore, heart disease is a disease that may occur suddenly by the above-described causes and has a very serious prognosis. Therefore, there is an urgent need to develop a method capable of diagnosing heart disease early and increasing therapeutic efficiency, and prior to this, it is very important to differentiate countermeasures for early diagnosis and treatment by predicting the onset of heart disease, and thus research thereon and technology development thereof are required.

Meanwhile, it is known that the number of microorganisms symbiotically living in the human body is 100 trillion which is 10 times the number of human cells, and the number of genes of microorganisms exceeds 100 times the number of human genes. A microbiota is a microbial community that includes bacteria, archaea, and eukaryotes present in a given habitat. The intestinal microbiota is known to play a vital role in human's physiological phenomena and significantly affect human health and diseases through interactions with human cells. Bacteria co-existing in human bodies secrete nanometer-sized vesicles to exchange information about genes, proteins, and the like with other cells. The mucous membranes form a physical barrier membrane that does not allow particles with the size of 200 nm or more to pass therethrough, and thus bacteria symbiotically living in the mucous membranes are unable to pass therethrough, but bacterial-derived extracellular vesicles have a size of approximately 100 nm or less and thus relatively freely pass through the mucous membranes and are absorbed into the human body.

Metagenomics, also called environmental genomics, may be analytics for metagenomic data obtained from samples collected from the environment, and collectively refers to a total genome of all microbiota in the natural environment in which microorganisms exist and was first used by Jo Handelsman in 1998 (Handelsman et al., 1998 Chem. Biol. 5, R245-249). Recently, the bacterial composition of human microbiota has been listed using a method based on 16s ribosomal RNA (16s rRNA) base sequences, and 16s ribosomal RNA analyzes sequences using a 454FLX titanium platform. In the onset of heart disease, however, identification of causative factors such as heart disease, particularly myocardial infarction, cardiomyopathy, variant angina, atrial fibrillation, and the like through metagenomic analysis of bacteria-derived vesicles isolated from a human-derived substance, such as blood, urine, or the like, and a method of diagnosing the heart disease have never been reported.

### DISCLOSURE

### Technical Problem

To diagnose heart disease such as myocardial infarction, cardiomyopathy, variant angina, atrial fibrillation, and the like, the inventors of the present invention extracted DNA from bacteria-derived vesicles using serum, which is a subject-derived sample, and performed metagenomic analysis on the extracted DNA, and, as a result, identified bacteria-derived extracellular vesicles capable of acting as a causative factor of heart disease, thus completing the present invention.

Therefore, it is an object of the present invention to provide a method of providing information for heart disease diagnosis by metagenomic analysis of bacteria-derived extracellular vesicles.

However, the technical goals of the present invention are not limited to the aforementioned goals, and other unmentioned technical goals will be clearly understood by those of ordinary skill in the art from the following description.

### Technical Solution

According to an aspect of the present invention, there is provided a method of providing information for heart disease diagnosis, comprising the following processes:

(a) extracting DNA from extracellular vesicles isolated from a subject sample;

(b) performing polymerase chain reaction (PCR) on the extracted DNA using a pair of primers having SEQ ID NO:1 and SEQ ID NO: 2; and

(c) comparing an increase or decrease in content of bacteria-derived extracellular vesicles of the subject sample with that of a normal individual-derived sample through sequencing of a product of the PCR.

The present invention also provides a method of diagnosing heart disease, comprising the following processes:

(a) extracting DNA from extracellular vesicles isolated from a subject sample;

(b) performing polymerase chain reaction (PCR) on the extracted DNA using a pair of primers having SEQ ID NO:1 and SEQ ID NO: 2; and

(c) comparing an increase or decrease in content of bacteria-derived extracellular vesicles of the subject sample with that of a normal individual-derived sample through sequencing of a product of the PCR.

The present invention also provides a method of predicting a risk for heart disease, comprising the following processes:

(a) extracting DNA from extracellular vesicles isolated from a subject sample;

(b) performing polymerase chain reaction (PCR) on the extracted DNA using a pair of primers having SEQ ID NO:1 and SEQ ID NO: 2; and

(c) comparing an increase or decrease in content of bacteria-derived extracellular vesicles of the subject sample with that of a normal individual-derived sample through sequencing of a product of the PCR.

In one embodiment of the present invention, the subject sample is blood, and the comparing may comprise comparing an increase or decrease in content of:

extracellular vesicles derived from one or more bacteria selected from the group consisting of the phylum Acidobacteria, the phylum Firmicutes, the phylum Crenarchaeota, the phylum Planctomycetes, the phylum Chloroflexi, the phylum Euryarchaeota, the phylum WS3, the phylum Nitrospirae, the phylum WPS-2, the phylum AD3, the phylum Verrucomicrobia, the phylum Gemmatimonadetes, the phylum Proteobacteria, the phylum TM7, and the phylum Cyanobacteria;

extracellular vesicles derived from one or more bacteria selected from the group consisting of the class Acidobacteriia, the class DA052, the class Methanomicrobia, the class Thaumarchaeota, the class Clostridia, the class Coriobacteriia, the class Betaproteobacteria, the class Ktedonobacteria, the class Planctomycetia, the class Solibacteres, the class Erysipelotrichi, the class Verrucomicrobiae, the class TM7-3, the class Bacteroidia, the class Phycisphaerae, the class MCG, the class Nitrospira, the class Pedosphaerae, the class Thermoleophilia, the class Saprospirae, the class PRR-12, the class Spartobacteria, the class Acidimicrobiia, the class TM1, the class Deltaproteobacteria, the class Anaerolineae, the class Thermoplasmata, the class Chthonomonadetes, the class Acidobacteria-6, the class Fusobacteriia, the class Fimbriimonadia, the class Actinobacteria, the class Flavobacteriia, and the class Chloroplast;

extracellular vesicles derived from one or more bacteria selected from the group consisting of the order Lactobacillales, the order Acidobacteriales, the order Enterobacteriales, the order Xanthomonadales, the order Clostridiales, the order Coriobacteriales, the order Ellin6513, the order Burkholderiales, the order Erysipelotrichales, the order Solibacterales, the order Verrucomicrobiales, the order Rhodospirillales, the order Gemmatales, the order Thermogemmatisporales, the order Saprospirales, the order Acidimicrobiales, the order Pedosphaerales, the order Bifidobacteriales, the order Chthoniobacterales, the order Solirubrobacterales, the order Syntrophobacterales, the order Bacteroidales, the order Nitrospirales, the order Ktedonobacterales, the order WD2101, the order iiil-15, the order Ellin329, the order Thiotrichales, the order Myxococcales, the order Stramenopiles, the order Vibrionales, the order Pseudomonadales, the order Bacillales, the order Sphingomonadales, the order Turicibacterales, the order Fimbriimonadales, the order Deinococcales, the order Pasteurellales, the order Flavobacteriales, the order Actinomycetales, the order Rhodobacterales, and the order Streptophyta;

extracellular vesicles derived from one or more bacteria selected from the group consisting of the family Koribacteraceae, the family Comamonadaceae, the family Enterobacteriaceae, the family Streptococcaceae, the family Coriobacteriaceae, the family Lachnospiraceae, the family Prevotellaceae, the family Ruminococcaceae, the family Xanthomonadaceae, the family Propionibacteriaceae, the family Hyphomicrobiaceae, the family Verrucomicrobiaceae, the family Solibacteraceae, the family Acidobacteriaceae, the family Erysipelotrichaceae, the family Ktedonobacteraceae, the family Thermogemmatisporaceae, the family Moraxellaceae, the family Veillonellaceae, the family Burkholderiaceae, the family Rhodospirillaceae, the family Bifidobacteriaceae, the family Gemmataceae, the family Streptomycetaceae, the family Chitinophagaceae, the family Brucellaceae, the family Rhizobiaceae, the family Chthoniobacteraceae, the family Sinobacteraceae, the family Conexibacteraceae, the family Oxalobacteraceae, the family Isosphaeraceae, the family Ellin515, the family Piscirickettsiaceae, the family Methylocystaceae, the family Pseudomonadaceae, the family Clostridiaceae, the family Sphingomonadaceae, the family Turicibacteraceae, the family Staphylococcaceae, the family Deinococcaceae, the family Methylobacteriaceae, the family Aerococcaceae, the family Fusobacteriaceae, the family Fimbriimonadaceae, the family Bacillaceae, the family Planococcaceae, the family Bacteroidaceae the family Weeksellaceae, the family Desulfovibrionaceae, the family Flavobacteriaceae, the family Rikenellaceae, the family S24-7, the family Pasteurellaceae, the family Rhodobacteraceae, the family Gordoniaceae, and the family Enterococcaceae; or

extracellular vesicles derived from one or more bacteria selected from the group consisting of the genus Delftia, the genus Agrobacterium, the genus Stenotrophomonas, the genus Faecalibacterium, the genus Candidatus Koribacter, the genus Akkermansia, the genus Streptococcus, the genus Salinispora, the genus Candidatus Solibacter, the genus Citrobacter, the genus Collinsella, the genus Burkholderia, the genus Coprococcus, the genus Rhodoplanes, the genus Acinetobacter, the genus Prevotella, the genus Propionibacterium, the genus Lactococcus, the genus Bifidobacterium, the genus Methanobacterium, the genus Micrococcus, the genus Methanocella, the genus Brevibacterium, the genus Streptacidiphilus, the genus Streptomyces, the genus Ochrobactrum, the genus Methanosaeta, the genus Lysinibacillus, the genus Cupriavidus, the genus Pseudomonas, the genus Clostridium, the genus Sphingomonas, the genus Staphylococcus, the genus Thermoanaerobacterium, the genus Neisseria, the genus Enhydrobacter, the genus Actinomyces, the genus Turicibacter, the genus Phascolarctobacterium, the genus Catenibacterium, the genus Bacillus, the genus Deinococcus, the genus Fusobacterium, the genus Adlercreutzia, the genus Chryseobacterium, the genus Enterococcus, the genus Alcanivorax, the genus Psychrobacter, the genus Bacteroides, the genus Geobacillus, the genus Haemophilus, the genus Veillonella, the genus Paracoccus, the genus Kocuria, the genus Halomonas, the genus Ruminococcus, and the genus Porphyromonas.

In one embodiment of the present invention, the heart disease may be myocardial infarction, cardiomyopathy, variant angina, or atrial fibrillation.

In another embodiment of the present invention, in process (c), myocardial infarction may be diagnosed by comparing an increase or decrease in content of extracellular vesicles derived from one or more bacteria selected from the group consisting of the phylum Acidobacteria, the phylum Firmicutes, the phylum Crenarchaeota, the phylum Planctomycetes, the phylum Chlorollexi, the phylum Euryarchaeota, the phylum WS3, the phylum Nitrospirae, the phylum WPS-2, and the phylum AD3.

In another embodiment of the present invention, in process (c), myocardial infarction may be diagnosed by comparing an increase or decrease in content of extracellular vesicles derived from one or more bacteria selected from the group consisting of the class Acidobacteriia, the class DA052, the class Methanomicrobia, the class Thaumarchaeota, the class Clostridia, the class Coriobacteriia, the class Betaproteobacteria, the class Ktedonobacteria, the class Planctomycetia, the class Solibacteres, the class Erysipelotrichi, the class Verrucomicrobiae, the class TM7-3, the class Bacteroidia, the class Phycisphaerae, the class MCG, the class Nitrospira, the class Pedosphaerae, the class Thermoleophilia, the class Saprospirae, the class PRR-12, the class Spartobacteria, the class Acidimicrobiia, the class TM1, the class Deltaproteobacteria, the class Anaerolineae, the class Thermoplasmata, the class Chthonomonadetes, and the class Acidobacteria-6.

In another embodiment of the present invention, in process (c), myocardial infarction may be diagnosed by comparing an increase or decrease in content of extracellular vesicles derived from one or more bacteria selected from the group consisting of the order Lactobacillales, the order Acidobacteriales, the order Enterobacteriales, the order Xanthomonadales, the order Clostridiales, the order Coriobacteriales, the order Ellin6513, the order Burkholderiales, the order Erysipelotrichales, the order Solibacterales, the order Verrucomicrobiales, the order Rhodospirillales, the order Gemmatales, the order Thermogemmatisporales, the order Saprospirales, the order Acidimicrobiales, the order Pedosphaerales, the order Bifidobacteriales, the order Chthoniobacterales, the order Solirubrobacterales, the order Syntrophobacterales, the order Bacteroidales, the order Nitrospirales, the order Ktedonobacterales, the order WD2101, the order iii1-15, the order Ellin329, the order Thiotrichales, the order Myxococcales, the order Stramenopiles, and the order Vibrionales.

In another embodiment of the present invention, in process (c), myocardial infarction may be diagnosed by comparing an increase or decrease in content of extracellular vesicles derived from one or more bacteria selected from the group consisting of the family Koribacteraceae, the family Comamonadaceae, the family Enterobacteriaceae, the family Streptococcaceae, the family Coriobacteriaceae, the family Lachnospiraceae, the family Prevotellaceae, the family Ruminococcaceae, the family Xanthomonadaceae, the family Propionibacteriaceae, the family Hyphomicrobiaceae, the family Verrucomicrobiaceae, the family Solibacteraceae, the family Acidobacteriaceae, the family Erysipelotrichaceae, the family Ktedonobacteraceae, the family Thermogemmatisporaceae, the family Moraxellaceae, the family Veillonellaceae, the family Burkholderiaceae, the family Rhodospirillaceae, the family Bifidobacteriaceae, the family Gemmataceae, the family Streptomycetaceae, the family Chitinophagaceae, the family Brucellaceae, the family Rhizobiaceae, the family Chthoniobacteraceae, the family Sinobacteraceae, the family Conexibacteraceae, the family Oxalobacteraceae, the family Isosphaeraceae, the family Ellin515, the family Piscirickettsiaceae, and the family Methylocystaceae.

In another embodiment of the present invention, in process (c), myocardial infarction may be diagnosed by comparing an increase or decrease in content of extracellular vesicles derived from one or more bacteria selected from the group consisting of the genus Delftia, the genus Agrobacterium, the genus Stenotrophomonas, the genus Faecalibacterium, the genus Candidatus Koribacter, the genus Akkermansia, the genus Streptococcus, the genus Salinispora, the genus Candidatus Solibacter, the genus Citrobacter, the genus Collinsella, the genus Burkholderia, the genus Coprococcus, the genus Rhodoplanes, the genus Acinetobacter, the genus Prevotella, the genus Propionibacterium, the genus Lactococcus, the genus Bifidobacterium, the genus Methanobacterium, the genus Micrococcus, the genus Methanocella, the genus Brevibacterium, the genus Streptacidiphilus, the genus Streptomyces, the genus Ochrobactrum, the genus Methanosaeta, the genus Lysinibacillus, and the genus Cupriavidus.

In another embodiment of the present invention, in process (c), cardiomyopathy may be diagnosed by comparing an increase or decrease in content of extracellular vesicles derived from one or more bacteria selected from the group consisting of the phylum Verrucomicrobia, the phylum Acidobacteria, the phylum Gemmatimonadetes, and the phylum Planctomycetes.

In another embodiment of the present invention, in process (c), cardiomyopathy may be diagnosed by comparing an increase or decrease in content of extracellular vesicles derived from one or more bacteria selected from the group consisting of the class Verrucomicrobiae, the class Fusobacteriia, the class Acidobacteriia, the class Planctomycetia, the class DA052, the class Deltaproteobacteria, and the class Acidimicrobiia.

In another embodiment of the present invention, in process (c), cardiomyopathy may be diagnosed by comparing an increase or decrease in content of extracellular vesicles derived from one or more bacteria selected from the group consisting of the order Pseudomonadales, the order Bacillales, the order Acidobacteriales, the order Sphingomonadales, the order Verrucomicrobiales, the order Turicibacterales, the order Acidimicrobiales, the order Ellin6513, the order Xanthomonadales, and the order Gemmatales.

In another embodiment of the present invention, in process (c), cardiomyopathy may be diagnosed by comparing an increase or decrease in content of extracellular vesicles derived from one or more bacteria selected from the group consisting of the family Pseudomonadaceae, the family Clostridiaceae, the family Comamonadaceae, the family Oxalobacteraceae, the family Moraxellaceae, the family Verrucomicrobiaceae, the family Koribacteraceae, the family Sphingomonadaceae, the family Turicibacteraceae, the family Xanthomonadaceae, the family Gemmataceae, and the family Staphylococcaceae.

In another embodiment of the present invention, in process (c), cardiomyopathy may be diagnosed by comparing an increase or decrease in content of extracellular vesicles derived from one or more bacteria selected from the group consisting of the genus Pseudomonas, the genus Clostridium, the genus Cupriavidus, the genus Acinetobacter, the genus Citrobacter, the genus Sphingomonas, the genus Candidatus Koribacter, the genus Staphylococcus, the genus Thermoanaerobacterium, the genus Micrococcus, the genus Akkermansia, the genus Neisseria, the genus Enhydrobacter, the genus Actinomyces, the genus Turicibacter, the genus Phascolarctobacterium, the genus Lactococcus, the genus Delftia, and the genus Stenotrophomonas.

In another embodiment of the present invention, in process (c), variant angina may be diagnosed by comparing an increase or decrease in content of extracellular vesicles derived from one or more bacteria selected from the group consisting of the phylum Verrucomicrobia, the phylum Acidobacteria, the phylum Planctomycetes, the phylum Gemmatimonadetes, the phylum Chloroflexi, and the phylum Euryarchaeota.

In another embodiment of the present invention, in process (c), variant angina may be diagnosed by comparing an increase or decrease in content of extracellular vesicles derived from one or more bacteria selected from the group consisting of the class Verrucomicrobiae, the class Acidobacteriia, the class Fimbriimonadia, the class Erysipelotrichi, the class Ktedonobacteria, and the class Deltaproteobacteria.

In another embodiment of the present invention, in process (c), variant angina may be diagnosed by comparing an increase or decrease in content of extracellular vesicles derived from one or more bacteria selected from the group consisting of the order Pseudomonadales, the order Erysipelotrichales, the order Fimbriimonadales, the order Acidobacteriales, the order Verrucomicrobiales, the order Xanthomonadales, the order Myxococcales, the order Deinococcales, and the order Rhodospirillales.

In another embodiment of the present invention, in process (c), variant angina may be diagnosed by comparing an increase or decrease in content of extracellular vesicles derived from one or more bacteria selected from the group consisting of the family Koribacteraceae, the family Oxalobacteraceae, the family Comamonadaceae, the family Moraxellaceae, the family Pseudomonadaceae, the family Hyphomicrobiaceae, the family Erysipelotrichaceae, the family Deinococcaceae, the family Clostridiaceae, the family Verrucomicrobiaceae, the family Sinobacteraceae, the family Rhodospirillaceae, the family Methylobacteriaceae, the family Aerococcaceae, the family Fusobacteriaceae, the family Fimbriimonadaceae, the family Bacillaceae, and the family Planococcaceae.

In another embodiment of the present invention, in process (c), variant angina may be diagnosed by comparing an increase or decrease in content of extracellular vesicles derived from one or more bacteria selected from the group consisting of the genus Citrobacter, the genus Acinetobacter, the genus Cupriavidus, the genus Clostridium, the genus Catenibacterium, the genus Pseudomonas, the genus Lactococcus, the genus Stenotrophomonas, the genus Akkermansia, the genus Bacillus, the genus Delftia, the genus Agrobacterium, the genus Deinococcus, the genus Fusobacterium, and the genus Adlercreutzia.

In another embodiment of the present invention, in process (c), atrial fibrillation may be diagnosed by comparing an increase or decrease in content of extracellular vesicles derived from one or more bacteria selected from the group consisting of the phylum Proteobacteria, the phylum TM7, the phylum Chloroflexi, the phylum Acidobacteria, and the phylum Cyanobacteria.

In another embodiment of the present invention, in process (c), atrial fibrillation may be diagnosed by comparing an increase or decrease in content of extracellular vesicles derived from one or more bacteria selected from the class Clostridia, the class Bacteroidia, the class Actinobacteria, the class Flavobacteriia, the class Erysipelotrichi, the class TM7-3, and the class Chloroplast.

In another embodiment of the present invention, in process (c), atrial fibrillation may be diagnosed by comparing an increase or decrease in content of extracellular vesicles derived from one or more bacteria selected from the group consisting of the order Pseudomonadales, the order Clostridiales, the order Bacteroidales, the order Enterobacteriales, the order Xanthomonadales, the order Bifidobacteriales, the order Pasteurellales, the order Flavobacteriales, the order Actinomycetales, the order Rhodobacterales, the order Coriobacteriales, the order Erysipelotrichales, and the order Streptophyta.

In another embodiment of the present invention, in process (c), atrial fibrillation may be diagnosed by comparing an increase or decrease in content of extracellular vesicles derived from one or more bacteria selected from the group consisting of the family Lachnospiraceae, the family Bacillaceae, the family Streptococcaceae, the family Bacteroidaceae, the family Moraxellaceae, the family Ruminococcaceae, the family Weeksellaceae, the family Bifidobacteriaceae, the family Clostridiaceae, the family Desulfovibrionaceae, the family Veillonellaceae, the family Coriobacteriaceae, the family Flavobacteriaceae, the family Rikenellaceae, the family S24-7, the family Pasteurellaceae, the family Rhodobacteraceae, the family Pseudomonadaceae, the family Gordoniaceae, and the family Enterococcaceae.

In another embodiment of the present invention, in process (c), atrial fibrillation may be diagnosed by comparing an increase or decrease in content of extracellular vesicles derived from one or more bacteria selected from the group consisting of the genus Acinetobacter, the genus Stenotrophomonas, the genus Chryseobacterium, the genus Enterococcus, the genus Pseudomonas, the genus Delftia, the genus Alcanivorax, the genus Psychrobacter, the genus Streptococcus, the genus Ochrobactrum, the genus Bifidobacterium, the genus Coprococcus, the genus Bacteroides, the genus Faecalibacterium, the genus Enhydrobacter, the genus Agrobacterium, the genus Citrobacter, the genus Prevotella, the genus Geobacillus, the genus Clostridium, the genus Bacillus, the genus Haemophilus, the genus Veillonella, the genus Actinomyces, the genus Paracoccus, the genus Kocuria, the genus Halomonas, the genus Micrococcus, the genus Ruminococcus, and the genus Porphyromonas.

In another embodiment of the present invention, the blood may be whole blood, serum, or plasma.

### Advantageous Effects

According to the present invention, a risk group for heart disease can be diagnosed early and predicted by diagnosing a causative factor of heart disease through metagenomic analysis of bacteria-derived extracellular vesicles from a human body-derived sample, and thus the onset of heart disease can be delayed or heart disease may be prevented through appropriate management, and, even after heart disease occurs, early diagnosis for heart disease can be implemented, thereby lowering a disease rate and increasing therapeutic effects.

In addition, patients diagnosed with heart disease are able to avoid exposure to causative factors predicted by metagenomic analysis, whereby the progression of heart disease is ameliorated, or recurrence of heart disease can be prevented.

### DESCRIPTION OF DRAWINGS

FIGS. 1A and 1B are views for evaluating the distribution pattern of extracellular vesicles (EVs) derived from bacteria in vivo. FIG. 1A illustrates images showing the distribution pattern of intestinal bacteria and EVs derived from bacteria per time (0 h, 5 min, 3 h, 6 h, and 12 h) after being orally administered to mice. FIG. 1B illustrates images showing the distribution pattern of gut bacteria and EVs derived from bacteria after being orally administered to mice and, after 12 hours, blood and various organs (heart, lung, liver, kidney, spleen, adipose tissue, and muscle) of the mice were extracted.

FIG. 2 illustrates distribution results of bacteria-derived EVs exhibiting significant diagnostic performance at a phylum level, after metagenomic analysis of bacteria-derived EVs isolated from myocardial infarction patient-derived blood and normal individual-derived blood.

FIG. 3 illustrates distribution results of bacteria-derived EVs exhibiting significant diagnostic performance at a class level, after metagenomic analysis of bacteria-derived EVs isolated from myocardial infarction patient-derived blood and normal individual-derived blood.

FIG. 4 illustrates distribution results of bacteria-derived EVs exhibiting significant diagnostic performance at an order level, after metagenomic analysis of bacteria-derived EVs isolated from myocardial infarction patient-derived blood and normal individual-derived blood.

FIG. 5 illustrates distribution results of bacteria-derived EVs exhibiting significant diagnostic performance at a family level, after metagenomic analysis of bacteria-derived EVs isolated from myocardial infarction patient-derived blood and normal individual-derived blood.

FIG. 6 illustrates distribution results of bacteria-derived EVs exhibiting significant diagnostic performance at a genus level, after metagenomic analysis of bacteria-derived EVs isolated from myocardial infarction patient-derived blood and normal individual-derived blood.

FIG. 7 illustrates distribution results of bacteria-derived EVs exhibiting significant diagnostic performance at a phylum level, after metagenomic analysis of bacteria-derived EVs isolated from cardiomyopathy patient-derived blood and normal individual-derived blood.

FIG. 8 illustrates distribution results of bacteria-derived EVs exhibiting significant diagnostic performance at a class level, after metagenomic analysis of bacteria-derived EVs isolated from cardiomyopathy patient-derived blood and normal individual-derived blood.

FIG. 9 illustrates distribution results of bacteria-derived EVs exhibiting significant diagnostic performance at an order level, after metagenomic analysis of bacteria-derived EVs isolated from cardiomyopathy patient-derived blood and normal individual-derived blood.

FIG. 10 illustrates distribution results of bacteria-derived EVs exhibiting significant diagnostic performance at a family level, after metagenomic analysis of bacteria-derived EVs isolated from cardiomyopathy patient-derived blood and normal individual-derived blood.

FIG. 11 illustrates distribution results of bacteria-derived EVs exhibiting significant diagnostic performance at a genus level, after metagenomic analysis of bacteria-derived EVs isolated from cardiomyopathy patient-derived blood and normal individual-derived blood.

FIG. 12 illustrates distribution results of bacteria-derived EVs exhibiting significant diagnostic performance at a phylum level, after metagenomic analysis of bacteria-derived EVs isolated from variant angina patient-derived blood and normal individual-derived blood.

FIG. 13 illustrates distribution results of bacteria-derived EVs exhibiting significant diagnostic performance at a class level, after metagenomic analysis of bacteria-derived EVs isolated from variant angina patient-derived blood and normal individual-derived blood.

FIG. 14 illustrates distribution results of bacteria-derived EVs exhibiting significant diagnostic performance at an order level, after metagenomic analysis of bacteria-derived EVs isolated from variant angina patient-derived blood and normal individual-derived blood.

FIG. 15 illustrates distribution results of bacteria-derived EVs exhibiting significant diagnostic performance at a family level, after metagenomic analysis of bacteria-derived EVs isolated from variant angina patient-derived blood and normal individual-derived blood.

FIG. 16 illustrates distribution results of bacteria-derived EVs exhibiting significant diagnostic performance at a genus level, after metagenomic analysis of bacteria-derived EVs isolated from variant angina patient-derived blood and normal individual-derived blood.

FIG. 17 illustrates distribution results of bacteria-derived EVs exhibiting significant diagnostic performance at a phylum level, after metagenomic analysis of bacteria-derived EVs isolated from atrial fibrillation patient-derived blood and normal individual-derived blood.

FIG. 18 illustrates distribution results of bacteria-derived EVs exhibiting significant diagnostic performance at a class level, after metagenomic analysis of bacteria-derived EVs isolated from atrial fibrillation patient-derived blood and normal individual-derived blood.

FIG. 19 illustrates distribution results of bacteria-derived EVs exhibiting significant diagnostic performance at an order level, after metagenomic analysis of bacteria-derived EVs isolated from atrial fibrillation patient-derived blood and normal individual-derived blood.

FIG. 20 illustrates distribution results of bacteria-derived EVs exhibiting significant diagnostic performance at a family level, after metagenomic analysis of bacteria-derived EVs isolated from atrial fibrillation patient-derived blood and normal individual-derived blood.

FIG. 21 illustrates distribution results of bacteria-derived EVs exhibiting significant diagnostic performance at a genus level, after metagenomic analysis of bacteria-derived EVs isolated from atrial fibrillation patient-derived blood and normal individual-derived blood.

### MODE OF THE INVENTION

The present invention relates to a method of diagnosing heart disease through bacterial metagenomic analysis. The inventors of the present invention extracted genes from bacteria-derived extracellular vesicles present in subject-derived samples, performed metagenomic analysis thereon, and identified bacteria-derived extracellular vesicles capable of acting as a causative factor of heart disease.

Thus, the present invention provides a method of providing information on heart disease diagnosis, the method including:

(a) extracting DNA from extracellular vesicles isolated from a subject sample;

(b) performing polymerase chain reaction (PCR) on the extracted DNA using a pair of primers having SEQ ID NOS: 1 and 2; and

(c) comparing an increase or decrease in content of bacteria-derived extracellular vesicles of the subject sample with that of a normal individual-derived sample through sequencing of a product of the PCR.

The term “prediction of a risk for heart disease” as used herein refers to determining whether a patient has a risk for heart disease, whether the risk for heart disease is relatively high, or whether heart disease has already occurred. The method of the present invention may be used to delay the onset of heart disease through special and appropriate care for a specific patient, which is a patient having a high risk for heart disease or prevent the onset of heart disease. In addition, the method may be clinically used to determine treatment by selecting the most appropriate treatment method through early diagnosis of heart disease.

In the present invention, the heart disease may be myocardial infarction, cardiomyopathy, variant angina, or atrial fibrillation.

The term “metagenome” as used herein refers to the total of genomes including all viruses, bacteria, fungi, and the like in isolated regions such as soil, the intestines of animals, and the like, and is mainly used as a concept of genomes that explains identification of many microorganisms at once using a sequencer to analyze non-cultured microorganisms. In particular, a metagenome does not refer to a genome of one species, but refers to a mixture of genomes, including genomes of all species of an environmental unit. This term originates from the view that, when defining one species in a process in which biology is advanced into omics, various species as well as existing one species functionally interact with each other to form a complete species. Technically, it is the subject of techniques that analyzes all DNAs and RNAs regardless of species using rapid sequencing to identify all species in one environment and verify interactions and metabolism. In the present invention, metagenomic analysis is performed using bacteria-derived extracellular vesicles isolated from blood.

In the present invention, the subject sample may be whole blood, serum, or plasma, but the present invention is not limited therto.

In an embodiment of the present invention, metagenomic analysis was performed on the bacteria-derived extracellular vesicles, and the bacteria-derived extracellular vesicles capable of acting as a cause of the onset of heart disease were actually identified by analysis at phylum, class, order, family, and genus levels.

More particularly, in one embodiment of the present invention, as a result of performing metagenomic analysis on bacteria-derived extracellular vesicles present in myocardial infarction patient-derived blood and normal individual-derived blood at a phylum level, the content of extracelluar vesicles derived from bacteria belonging to the phylum Acidobacteria, the phylum Firmicutes, the phylum Crenarchaeota, the phylum Planctomycetes, the phylum Chlorollexi, the phylum Euryarchaeota, the phylum WS3, the phylum Nitrospirae, the phylum WPS-2, and the phylum AD3 was significantly different between myocardial infarction patients and normal individuals (see Example 4).

More particularly, in one embodiment of the present invention, as a result of performing metagenomic analysis on bacteria-derived extracellular vesicles present in myocardial infarction patient-derived blood and normal individual-derived blood at a class level, the content of extracelluar vesicles derived from bacteria belonging to the class Acidobacteriia, the class DA052, the class Methanomicrobia, the class Thaumarchaeota, the class Clostridia, the class Coriobacteriia, the class Betaproteobacteria, the class Ktedonobacteria, the class Planctomycetia, the class Solibacteres, the class Erysipelotrichi, the class Verrucomicrobiae, the class TM7-3, the class Bacteroidia, the class Phycisphaerae, the class MCG, the class Nitrospira, the class Pedosphaerae, the class Thermoleophilia, the class Saprospirae, the class PRR-12, the class Spartobacteria, the class Acidimicrobiia, the class TM1, the class Deltaproteobacteria, the class Anaerolineae, the class Thermoplasmata, the class Chthonomonadetes, and the class Acidobacteria-6 was significantly different between myocardial infarction patients and normal individuals (see Example 4).

More particularly, in one embodiment of the present invention, as a result of performing metagenomic analysis on bacteria-derived extracellular vesicles present in myocardial infarction patient-derived blood and normal individual-derived blood at an order level, the content of extracelluar vesicles derived from bacteria belonging to the order Lactobacillales, the order Acidobacteriales, the order Enterobacteriales, the order Xanthomonadales, the order Clostridiales, the order Coriobacteriales, the order Ellin6513, the order Burkholderiales, the order Erysipelotrichales, the order Solibacterales, the order Verrucomicrobiales, the order Rhodospirillales, the order Gemmatales, the order Thermogemmatisporales, the order Saprospirales, the order Acidimicrobiales, the order Pedosphaerales, the order Bifidobacteriales, the order Chthoniobacterales, the order Solirubrobacterales, the order Syntrophobacterales, the order Bacteroidales, the order Nitrospirales, the order Ktedonobacterales, the order WD2101, the order iiil-15, the order Ellin329, the order Thiotrichales, the order Myxococcales, the order Stramenopiles, and the order Vibrionales was significantly different between myocardial infarction patients and normal individuals (see Example 4).

More particularly, in one embodiment of the present invention, as a result of performing metagenomic analysis on bacteria-derived extracellular vesicles present in myocardial infarction patient-derived blood and normal individual-derived blood at a family level, the content of extracelluar vesicles derived from bacteria belonging to the family Koribacteraceae, the family Comamonadaceae, the family Enterobacteriaceae, the family Streptococcaceae, the family Coriobacteriaceae, the family Lachnospiraceae, the family Prevotellaceae, the family Ruminococcaceae, the family Xanthomonadaceae, the family Propionibacteriaceae, the family Hyphomicrobiaceae, the family Verrucomicrobiaceae, the family Solibacteraceae, the family Acidobacteriaceae, the family Erysipelotrichaceae, the family Ktedonobacteraceae, the family Thermogemmatisporaceae, the family Moraxellaceae, the family Veillonellaceae, the family Burkholderiaceae, the family Rhodospirillaceae, the family Bifidobacteriaceae, the family Gemmataceae, the family Streptomycetaceae, the family Chitinophagaceae, the family Brucellaceae, the family Rhizobiaceae, the family Chthoniobacteraceae, the family Sinobacteraceae, the family Conexibacteraceae, the family Oxalobacteraceae, the family Isosphaeraceae, the family Ellin515, the family Piscirickettsiaceae, and the family Methylocystaceae was significantly different between myocardial infarction patients and normal individuals (see Example 4).

More particularly, in one embodiment of the present invention, as a result of performing metagenomic analysis on bacteria-derived extracellular vesicles present in myocardial infarction patient-derived blood and normal individual-derived blood at a genus level, the content of extracelluar vesicles derived from bacteria belonging to the genus Delftia, the genus Agrobacterium, the genus Stenotrophomonas, the genus Faecalibacterium, the genus Candidatus Koribacter, the genus Akkermansia, the genus Streptococcus, the genus Salinispora, the genus Candidatus Solibacter, the genus Citrobacter, the genus Collinsella, the genus Burkholderia, the genus Coprococcus, the genus Rhodoplanes, the genus Acinetobacter, the genus Prevotella, the genus Propionibacterium, the genus Lactococcus, the genus Bifidobacterium, the genus Methanobacterium, the genus Micrococcus, the genus Methanocella, the genus Brevibacterium, the genus Streptacidiphilus, the genus Streptomyces, the genus Ochrobactrum, the genus Methanosaeta, the genus Lysinibacillus, and the genus Cupriavidus was significantly different between myocardial infarction patients and normal individuals (see Example 4).

More particularly, in one embodiment of the present invention, as a result of performing metagenomic analysis on bacteria-derived extracellular vesicles present in cardiomyopathy patient-derived blood and normal individual-derived blood at a phylum level, the content of extracelluar vesicles derived from bacteria belonging to the phylum Verrucomicrobia, the phylum Acidobacteria, the phylum Gemmatimonadetes, and the phylum Planctomycetes was significantly different between cardiomyopathy patients and normal individuals (see Example 5).

More particularly, in one embodiment of the present invention, as a result of performing metagenomic analysis on bacteria-derived extracellular vesicles present in cardiomyopathy patient-derived blood and normal individual-derived blood at a class level, the content of extracelluar vesicles derived from bacteria belonging to the class Verrucomicrobiae, the class Fusobacteriia, the class Acidobacteriia, the class Planctomycetia, the class DA052, the class Deltaproteobacteria, and the class Acidimicrobiia was significantly different between cardiomyopathy patients and normal individuals (see Example 5).

More particularly, in one embodiment of the present invention, as a result of performing metagenomic analysis on bacteria-derived extracellular vesicles present in cardiomyopathy patient-derived blood and normal individual-derived blood at an order level, the content of extracelluar vesicles derived from bacteria belonging to the order Pseudomonadales, the order Bacillales, the order Acidobacteriales, the order Sphingomonadales, the order Verrucomicrobiales, the order Turicibacterales, the order Acidimicrobiales, the order Ellin6513, the order Xanthomonadales, and the order Gemmatales was significantly different between cardiomyopathy patients and normal individuals (see Example 5).

More particularly, in one embodiment of the present invention, as a result of performing metagenomic analysis on bacteria-derived extracellular vesicles present in cardiomyopathy patient-derived blood and normal individual-derived blood at a family level, the content of extracelluar vesicles derived from bacteria belonging to the family Pseudomonadaceae, the family Clostridiaceae, the family Comamonadaceae, the family Oxalobacteraceae, the family Moraxellaceae, the family Verrucomicrobiaceae, the family Koribacteraceae, the family Sphingomonadaceae, the family Turicibacteraceae, the family Xanthomonadaceae, the family Gemmataceae, and the family Staphylococcaceae was significantly different between cardiomyopathy patients and normal individuals (see Example 5).

More particularly, in one embodiment of the present invention, as a result of performing metagenomic analysis on bacteria-derived extracellular vesicles present in cardiomyopathy patient-derived blood and normal individual-derived blood at a genus level, the content of extracelluar vesicles derived from bacteria belonging to the genus Pseudomonas, the genus Clostridium, the genus Cupriavidus, the genus Acinetobacter, the genus Citrobacter, the genus Sphingomonas, the genus Candidatus Koribacter, the genus Staphylococcus, the genus Thermoanaerobacterium, the genus Micrococcus, the genus Akkermansia, the genus Neisseria, the genus Enhydrobacter, the genus Actinomyces, the genus Turicibacter, the genus Phascolarctobacterium, the genus Lactococcus, the genus Delftia, and the genus Stenotrophomonas was significantly different between cardiomyopathy patients and normal individuals (see Example 5).

More particularly, in one embodiment of the present invention, as a result of performing metagenomic analysis on bacteria-derived extracellular vesicles present in variant angina patient-derived blood and normal individual-derived blood at a phylum level, the content of extracelluar vesicles derived from bacteria belonging to the phylum Verrucomicrobia, the phylum Acidobacteria, the phylum Planctomycetes, the phylum Gemmatimonadetes, the phylum Chloroflexi, and the phylum Euryarchaeota was significantly different between variant angina patients and normal individuals (see Example 6).

More particularly, in one embodiment of the present invention, as a result of performing metagenomic analysis on bacteria-derived extracellular vesicles present in variant angina patient-derived blood and normal individual-derived blood at a class level, the content of extracelluar vesicles derived from bacteria belonging to the class Verrucomicrobiae, the class Acidobacteriia, the class Fimbriimonadia, the class Erysipelotrichi, the class Ktedonobacteria, and the class Deltaproteobacteria was significantly different between variant angina patients and normal individuals (see Example 6).

More particularly, in one embodiment of the present invention, as a result of performing metagenomic analysis on bacteria-derived extracellular vesicles present in variant angina patient-derived blood and normal individual-derived blood at an order level, the content of extracelluar vesicles derived from bacteria belonging to the order Pseudomonadales, the order Erysipelotrichales, the order Fimbriimonadales, the order Acidobacteriales, the order Verrucomicrobiales, the order Xanthomonadales, the order Myxococcales, the order Deinococcales, and the order Rhodospirillales was significantly different between variant angina patients and normal individuals (see Example 6).

More particularly, in one embodiment of the present invention, as a result of performing metagenomic analysis on bacteria-derived extracellular vesicles present in variant angina patient-derived blood and normal individual-derived blood at a family level, the content of extracelluar vesicles derived from bacteria belonging to the family Koribacteraceae, the family Oxalobacteraceae, the family Comamonadaceae, the family Moraxellaceae, the family Pseudomonadaceae, the family Hyphomicrobiaceae, the family Erysipelotrichaceae, the family Deinococcaceae, the family Clostridiaceae, the family Verrucomicrobiaceae, the family Sinobacteraceae, the family Rhodospirillaceae, the family Methylobacteriaceae, the family Aerococcaceae, the family Fusobacteriaceae, the family Fimbriimonadaceae, the family Bacillaceae, and the family Planococcaceae was significantly different between variant angina patients and normal individuals (see Example 6).

More particularly, in one embodiment of the present invention, as a result of performing metagenomic analysis on bacteria-derived extracellular vesicles present in variant angina patient-derived blood and normal individual-derived blood at a genus level, the content of extracelluar vesicles derived from bacteria belonging to the genus Citrobacter, the genus Acinetobacter, the genus Cupriavidus, the genus Clostridium, the genus Catenibacterium, the genus Pseudomonas, the genus Lactococcus, the genus Stenotrophomonas, the genus Akkermansia, the genus Bacillus, the genus Delftia, the genus Agrobacterium, the genus Deinococcus, the genus Fusobacterium, and the genus Adlercreutzia was significantly different between variant angina patients and normal individuals (see Example 6).

More particularly, in one embodiment of the present invention, as a result of performing metagenomic analysis on bacteria-derived extracellular vesicles present in atrial fibrillation patient-derived blood and normal individual-derived blood at a phylum level, the content of extracelluar vesicles derived from bacteria belonging to the phylum Proteobacteria, the phylum TM7, the phylum Chloroflexi, the phylum Acidobacteria, and the phylum Cyanobacteria was significantly different between atrial fibrillation patients and normal individuals (see Example 7).

More particularly, in one embodiment of the present invention, as a result of performing metagenomic analysis on bacteria-derived extracellular vesicles present in atrial fibrillation patient-derived blood and normal individual-derived blood at a class level, the content of extracelluar vesicles derived from bacteria belonging to the class Clostridia, the class Bacteroidia, the class Actinobacteria, the class Flavobacteriia, the class Erysipelotrichi, the class TM7-3, and the class Chloroplast was significantly different between atrial fibrillation patients and normal individuals (see Example 7).

More particularly, in one embodiment of the present invention, as a result of performing metagenomic analysis on bacteria-derived extracellular vesicles present in atrial fibrillation patient-derived blood and normal individual-derived blood at an order level, the content of extracelluar vesicles derived from bacteria belonging to the order Pseudomonadales, the order Clostridiales, the order Bacteroidales, the order Enterobacteriales, the order Xanthomonadales, the order Bifidobacteriales, the order Pasteurellales, the order Flavobacteriales, the order Actinomycetales, the order Rhodobacterales, the order Coriobacteriales, the order Erysipelotrichales, and the order Streptophyta was significantly different between atrial fibrillation patients and normal individuals (see Example 7).

More particularly, in one embodiment of the present invention, as a result of performing metagenomic analysis on bacteria-derived extracellular vesicles present in atrial fibrillation patient-derived blood and normal individual-derived blood at a family level, the content of extracelluar vesicles derived from bacteria belonging to the family Lachnospiraceae, the family Bacillaceae, the family Streptococcaceae, the family Bacteroidaceae, the family Moraxellaceae, the family Ruminococcaceae, the family Weeksellaceae, the family Bifidobacteriaceae, the family Clostridiaceae, the family Desulfovibrionaceae, the family Veillonellaceae, the family Coriobacteriaceae, the family Flavobacteriaceae, the family Rikenellaceae, the family S24-7, the family Pasteurellaceae, the family Rhodobacteraceae, the family Pseudomonadaceae, the family Gordoniaceae, and the family Enterococcaceae was significantly different between atrial fibrillation patients and normal individuals (see Example 7).

More particularly, in one embodiment of the present invention, as a result of performing metagenomic analysis on bacteria-derived extracellular vesicles present in atrial fibrillation patient-derived blood and normal individual-derived blood at a genus level, the content of extracelluar vesicles derived from bacteria belonging to the genus Acinetobacter, the genus Stenotrophomonas, the genus Chryseobacterium, the genus Enterococcus, the genus Pseudomonas, the genus Delftia, the genus Alcanivorax, the genus Psychrobacter, the genus Streptococcus, the genus Ochrobactrum, the genus Bifidobacterium, the genus Coprococcus, the genus Bacteroides, the genus Faecalibacterium, the genus Enhydrobacter, the genus Agrobacterium, the genus Citrobacter, the genus Prevotella, the genus Geobacillus, the genus Clostridium, the genus Bacillus, the genus Haemophilus, the genus Veillonella, the genus Actinomyces, the genus Paracoccus, the genus Kocuria, the genus Halomonas, the genus Micrococcus, the genus Ruminococcus, and the genus Porphyromonas was significantly different between atrial fibrillation patients and normal individuals (see Example 7).

From the above-described example results, it was confirmed that bacteria-derived extracellular vesicles exhibiting a significant change in content in patients with myocardial infarction, cardiomyopathy, variant angina, or atrial fibrillation compared to normal individuals, were identified by performing metagenomic analysis on extracellular vesicles isolated from blood, and heart disease may be diagnosed by analyzing an increase or decrease in the content of bacteria-derived vesicles at each level through metagenomic analysis.

Hereinafter, the present invention will be described with reference to exemplary examples to aid in understanding of the present invention. However, these examples are provided only for illustrative purposes and are not intended to limit the scope of the present invention.

### EXAMPLES

### Example 1. Analysis of In Vivo Absorption, Distribution, and Excretion Patterns of Intestinal Bacteria and Bacteria-Derived Extracellular Vesicles

To evaluate whether intestinal bacteria and bacteria-derived extracellular vesicles are systematically absorbed through the gastrointestinal tract, an experiment was conducted using the following method. More particularly, 50 μg of each of Pseudomonas panacis, which is an intestinal bacterium, and the bacteria-derived extracellular vesicles (EVs), labeled with fluorescence, were orally administered to the gastrointestinal tracts of mice, and fluorescence was measured at 0 h, and after 5 min, 3 h, 6 h, and 12 h.

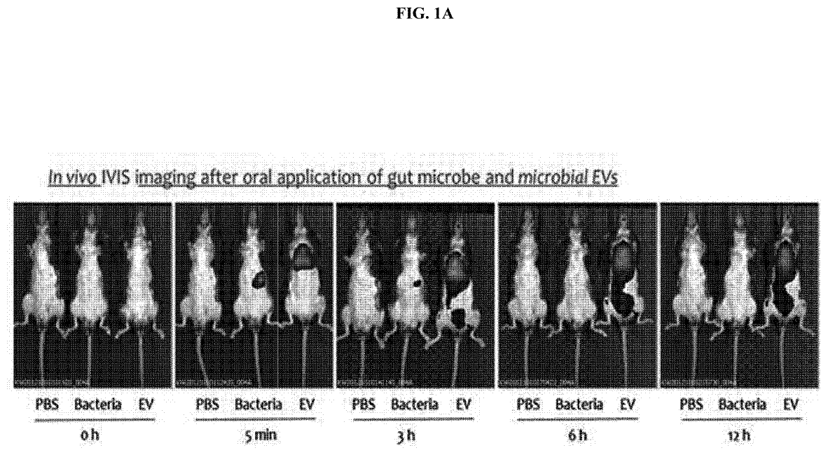

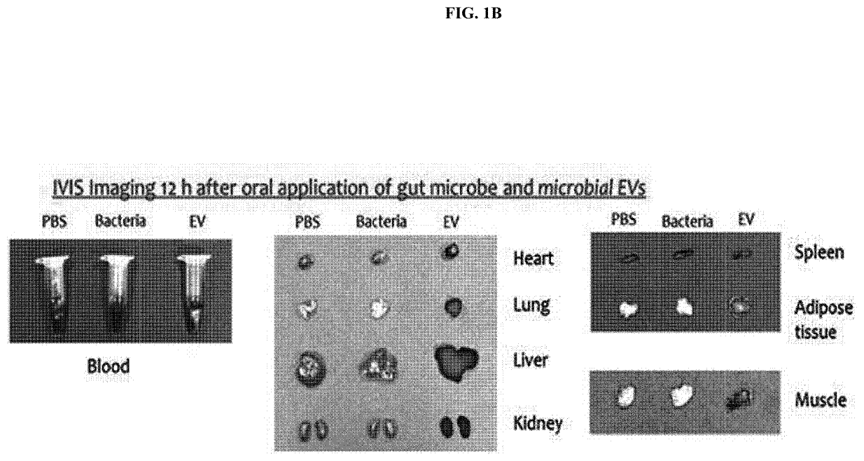

As a result of observing the entire images of mice, as illustrated in FIG. 1A, the bacteria were not systematically absorbed when the bacteria was administered, while the bacteria-derived EVs were systematically absorbed at 5 min after administration, and, at 30 minutes after administration, fluorescence was strongly observed in the bladder, from which it was confirmed that the EVs were excreted via the urinary system through blood, and were present in the bodies up to 12 h after administration.

In addition to the above results, after intestinal bacteria-derived extracellular vesicles were systematically absorbed, to evaluate a pattern of invasion of the intestinal bacteria-derived EVs into various organs, 50 μg of each of the bacteria and bacteria-derived EVs, labeled with fluorescence, were administered using the same method as that used above, and then, at 12 h after administration, blood, the heart, the lungs, the liver, the kidneys, the spleen, adipose tissue, and muscle were extracted from each mouse. As a result of observing fluorescence in the extracted tissues, as illustrated in FIG. 1B, it was confirmed that the intestinal bacteria were not absorbed into each organ, while the intestinal bacteria-derived EVs were distributed in the blood, heart, lungs, liver, kidneys, spleen, adipose tissue, and muscle.

### Example 2. Vesicle Isolation and DNA Extraction from Blood

First, blood was added to a 10 ml tube and centrifuged at 3,500×g and 4□ for 10 min to precipitate a suspension, and only a supernatant was then placed in a new 10 ml tube. Bacteria and impurities were removed using a 0.22 μm filter, and then placed in centripreigugal filters (50 kD) and centrifuged at 1500×g and 4□ for 15 min to discard materials with a smaller size than 50 kD, and then concentrated to 10 ml. Once again, bacteria and impurities were removed therefrom using a 0.22 μm filter, and then the resulting concentrate was subjected to ultra-high speed centrifugation at 150,000×g and 4□ to remove a supernatant, and the agglomerated pellet was dissolved with phosphate-buffered saline (PBS).

100 μl of the vesicles isolated according to the above-described method was boiled at 100□ to allow the internal DNA to come out of the lipid and then cooled on ice for 5 minutes. Next, the resulting vesicles were centrifuged at 10,000×g and 4□ for 30 minutes to remove the remaining suspension, only the supernatant was collected, and then the amount of DNA extracted was quantified using a NanoDrop sprectrophotometer. In addition, to verify whether bacteria-derived DNA was present in the extracted DNA, PCR was performed using 16s rDNA primers shown in Table 1 below to verify whether bacteria-derived DNA was present in the extracted DNA.

TABLE 1

| primer | | sequence | SEQ ID NO. |
|---|---|---|---|
| 16S rDNA | 16S_V3_F | 5′-TCGTCGGCAGCGTCAGATGTGTATAAG | 1 |
| | | AGACAGCCTACGGGNGGCWGCAG-3′ | |
| | 16S_V4_R | 5′-GTCTCGTGGGCTCGGAGATGTGTATAA | 2 |
| | | GAGACAGGACTACHVGGGTATCTAATCC-3 | |

### Example 3. 16S rDNA Sequencing Using Extracelluar Vesicle-Derived DNA Isolated from Blood

DNA was extracted using the same method as that used in Example 2, and then PCR was performed thereon using 16S rDNA primers shown in Table 1 to amplify DNA, followed by sequencing (Illumina MiSeq sequencer). The results were output as standard flowgram format (SFF) files, and the SFF files were converted into sequence files (.fasta) and nucleotide quality score files using GS FLX software (v2.9), and then credit rating for reads was identified, and portions with a window (20 bps) average base call accuracy of less than 99% (Phred score <20) were removed. After removing the low-quality portions, only reads having a length of 300 bps or more were used (Sickle version 1.33), and, for operational taxonomy unit (OTU) analysis, clustering was performed using UCLUST and USEARCH according to sequence similarity. In particular, clustering was performed based on sequence similarity values of 94% for genus, 90% for family, 85% for order, 80% for class, and 75% for phylum, and phylum, class, order, family, and genus levels of each OTU were classified, and bacteria with a sequence similarity of 97% or more were analyzed (QIIME) using 16S RNA sequence databases (108,453 sequences) of BLASTN and GreenGenes.

### Example 4. Myocardial infarction Diagnostic Model Based on Meta2enomic Profiling of Bacteria-Derived EVs

EVs were isolated from blood samples of 57 ST elevation myocardial infarction (STEMI) patients and 163 normal individuals, the two groups matched in age and gender, and then metagenomic sequencing was performed thereon using the method of Example 3. For the development of a diagnostic model, first, a strain exhibiting a p value of less than 0.05 between two groups in a t-test and a difference of two-fold or more between two groups was selected, and then an area under curve (AUC), accuracy, sensitivity, and specificity, which are diagnostic performance indexes, were calculated by logistic regression analysis.

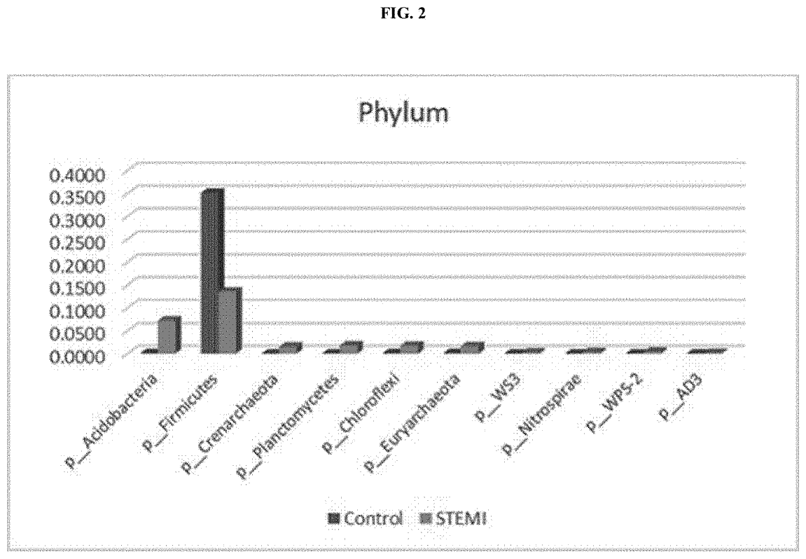

As a result of analyzing bacteria-derived EVs in blood at a phylum level, a diagnostic model developed using, as a biomarker, one or more bacteria from the phylum Acidobacteria, the phylum Firmicutes, the phylum Crenarchaeota, the phylum Planctomycetes, the phylum Chloroflexi, the phylum Euryarchaeota, the phylum WS3, the phylum Nitrospirae, the phylum WPS-2, and the phylum AD3 exhibited significant diagnostic performance for STEMI (see Table 2 and FIG. 2).

TABLE 2

| | | | | | | | Training Set | | | | Test Set | | | |
|---|---|---|---|---|---|---|---|---|---|---|---|---|---|---|
| | | | | | t-test | | | | Sen- | Spec- | | | Sen- | Spec- |
| | Control | | STEMI | | p- | Ra- | | Acu- | sitiv- | ific- | | Acu- | sitiv- | ific- |
| Taxon | Mean | SD | Mean | SD | value | tio | AUC | rracy | ity | ity | AUC | rracy | ity | ity |
| p_Firmicutes | 0.3515 | 0.1050 | 0.1354 | 0.0652 | 0.0000 | 0.39 | 0.97 | 0.93 | 0.95 | 0.88 | 0.94 | 0.85 | 0.86 | 0.81 |
| p_Euryarchaeota | 0.0013 | 0.0032 | 0.0160 | 0.0179 | 0.0000 | 11.95 | 0.81 | 0.87 | 0.98 | 0.56 | 0.77 | 0.85 | 0.96 | 0.50 |
| p_Chloroflexi | 0.0010 | 0.0036 | 0.0172 | 0.0169 | 0.0000 | 16.82 | 0.84 | 0.87 | 0.98 | 0.56 | 0.86 | 0.91 | 0.98 | 0.69 |
| p_WPS-2 | 0.0002 | 0.0018 | 0.0042 | 0.0080 | 0.0005 | 20.81 | 0.69 | 0.79 | 0.99 | 0.24 | 0.77 | 0.83 | 0.96 | 0.44 |
| p_AD3 | 0.0000 | 0.0003 | 0.0016 | 0.0042 | 0.0072 | 35.51 | 0.67 | 0.77 | 1.00 | 0.15 | 0.58 | 0.79 | 1.00 | 0.13 |
| p_Planctomycetes | 0.0004 | 0.0017 | 0.0178 | 0.0173 | 0.0000 | 44.17 | 0.90 | 0.91 | 0.97 | 0.73 | 0.92 | 0.89 | 0.96 | 0.69 |
| p_Acidobacteria | 0.0009 | 0.0027 | 0.0721 | 0.0402 | 0.0000 | 77.14 | 0.99 | 0.97 | 0.99 | 0.93 | 1.00 | 0.98 | 1.00 | 0.94 |
| p_Nitrospirae | 0.0000 | 0.0004 | 0.0034 | 0.0069 | 0.0006 | 94.81 | 0.72 | 0.81 | 0.99 | 0.32 | 0.60 | 0.79 | 1.00 | 0.13 |
| p_Crenarchaeota | 0.0000 | 0.0000 | 0.0150 | 0.0208 | 0.0000 | >100 | 0.92 | 0.95 | 1.00 | 0.78 | 0.92 | 0.92 | 1.00 | 0.75 |
| p_WS3 | 0.0000 | 0.0000 | 0.0026 | 0.0063 | 0.0000 | >100 | 0.72 | 0.85 | 1.00 | 0.38 | 0.65 | 0.76 | 1.00 | 0.20 |

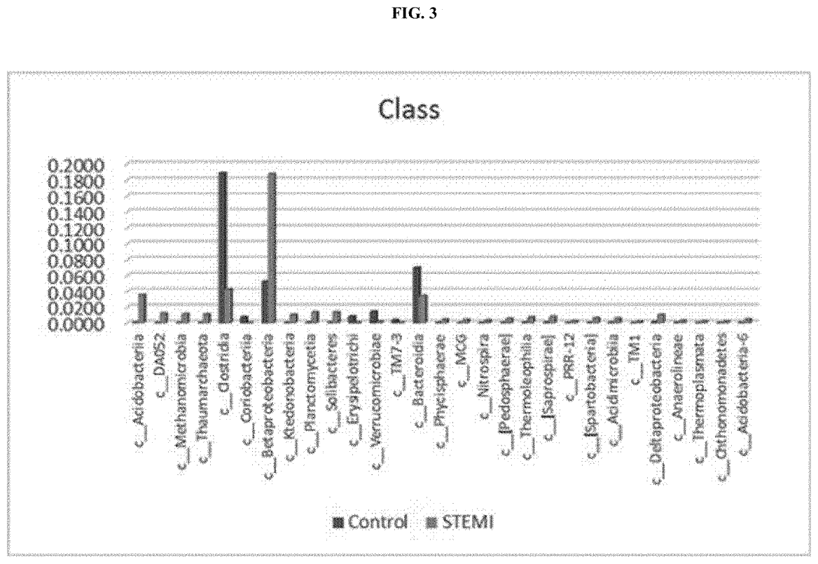

As a result of analyzing bacteria-derived EVs in blood at a class level, a diagnostic model developed using, as a biomarker, one or more bacteria from the class Acidobacteriia, the class DA052, the class Methanomicrobia, the class Thaumarchaeota, the class Clostridia, the class Coriobacteriia, the class Betaproteobacteria, the class Ktedonobacteria, the class Planctomycetia, the class Solibacteres, the class Erysipelotrichi, the class Verrucomicrobiae, the class TM7-3, the class Bacteroidia, the class Phycisphaerae, the class MCG, the class Nitrospira, the class Pedosphaerae, the class Thermoleophilia, the class Saprospirae, the class PRR-12, the class Spartobacteria, the class Acidimicrobiia, the class TM1, the class Deltaproteobacteria, the class Anaerolineae, the class Thermoplasmata, the class Chthonomonadetes, and the class Acidobacteria-6 exhibited significant diagnostic performance for STEMI (see Table 3 and FIG. 3).

TABLE 3

| | | | | | | | Training Set | | | | Test Set | | | |
|---|---|---|---|---|---|---|---|---|---|---|---|---|---|---|
| | | | | | t-test | | | | Sen- | Spec- | | | Sen- | Spec- |
| | Control | | STEMI | | p- | Ra- | | Acu- | sitiv- | ific- | | Acu- | sitiv- | ific- |
| Taxon | Mean | SD | Mean | SD | value | tio | AUC | rracy | ity | ity | AUC | rracy | ity | ity |
| c_Coriobacteriia | 0.0075 | 0.0091 | 0.0001 | 0.0003 | 0.0000 | 0.01 | 0.90 | 0.81 | 0.80 | 0.81 | 0.86 | 0.86 | 0.88 | 0.80 |
| c_Erysipelotrichi | 0.0082 | 0.0128 | 0.0006 | 0.0021 | 0.0000 | 0.07 | 0.82 | 0.76 | 0.96 | 0.24 | 0.87 | 0.79 | 0.96 | 0.20 |
| c_Verrucomicrobiae | 0.0143 | 0.0181 | 0.0017 | 0.0041 | 0.0000 | 0.12 | 0.81 | 0.77 | 0.81 | 0.64 | 0.90 | 0.82 | 0.86 | 0.67 |
| c_TM7-3 | 0.0037 | 0.0071 | 0.0006 | 0.0025 | 0.0000 | 0.16 | 0.81 | 0.73 | 1.00 | 0.00 | 0.68 | 0.77 | 1.00 | 0.00 |
| c_Clostridia | 0.1885 | 0.1143 | 0.0425 | 0.0313 | 0.0000 | 0.23 | 0.91 | 0.85 | 0.89 | 0.80 | 0.94 | 0.86 | 0.86 | 0.87 |
| c_Bacteroidia | 0.0695 | 0.0488 | 0.0343 | 0.0244 | 0.0000 | 0.49 | 0.79 | 0.77 | 0.92 | 0.36 | 0.67 | 0.74 | 0.88 | 0.27 |
| c_Betaproteobacteria | 0.0523 | 0.0422 | 0.1874 | 0.1471 | 0.0000 | 3.58 | 0.88 | 0.82 | 0.95 | 0.50 | 0.94 | 0.86 | 1.00 | 0.40 |
| c_Deltaproteobacteria | 0.0011 | 0.0023 | 0.0104 | 0.0149 | 0.0000 | 9.60 | 0.67 | 0.81 | 0.96 | 0.40 | 0.84 | 0.86 | 0.98 | 0.47 |
| c_[Saprospirae] | 0.0006 | 0.0023 | 0.0079 | 0.0166 | 0.0019 | 12.60 | 0.73 | 0.82 | 0.97 | 0.43 | 0.75 | 0.83 | 0.94 | 0.47 |
| c_Thermoleophilia | 0.0005 | 0.0035 | 0.0074 | 0.0105 | 0.0000 | 13.97 | 0.75 | 0.80 | 0.98 | 0.31 | 0.87 | 0.86 | 1.00 | 0.40 |
| c_Acidimicrobiia | 0.0003 | 0.0013 | 0.0056 | 0.0091 | 0.0000 | 20.03 | 0.70 | 0.82 | 0.97 | 0.40 | 0.73 | 0.82 | 1.00 | 0.20 |
| c_Acidobacteria-6 | 0.0002 | 0.0014 | 0.0045 | 0.0082 | 0.0003 | 25.38 | 0.62 | 0.78 | 0.99 | 0.21 | 0.72 | 0.85 | 1.00 | 0.33 |
| c_Planctomycetia | 0.0003 | 0.0015 | 0.0136 | 0.0154 | 0.0000 | 41.25 | 0.87 | 0.88 | 0.97 | 0.64 | 0.93 | 0.88 | 0.96 | 0.60 |
| c_[Spartobacteria] | 0.0001 | 0.0009 | 0.0062 | 0.0116 | 0.0003 | 49.89 | 0.72 | 0.81 | 0.98 | 0.33 | 0.86 | 0.86 | 1.00 | 0.40 |
| c_Anaerolineae | 0.0001 | 0.0005 | 0.0030 | 0.0065 | 0.0015 | 51.96 | 0.66 | 0.78 | 1.00 | 0.19 | 0.87 | 0.82 | 0.98 | 0.27 |
| c_[Pedosphaerae] | 0.0001 | 0.0006 | 0.0055 | 0.0084 | 0.0000 | 56.31 | 0.75 | 0.83 | 0.98 | 0.43 | 0.84 | 0.86 | 1.00 | 0.40 |
| c_Phycisphaerae | 0.0001 | 0.0006 | 0.0042 | 0.0087 | 0.0008 | 56.63 | 0.78 | 0.82 | 0.99 | 0.36 | 0.75 | 0.79 | 0.96 | 0.20 |
| c_Solibacteres | 0.0002 | 0.0016 | 0.0135 | 0.0155 | 0.0000 | 56.64 | 0.86 | 0.87 | 0.99 | 0.55 | 0.69 | 0.89 | 1.00 | 0.53 |
| c_Nitrospira | 0.0000 | 0.0004 | 0.0034 | 0.0069 | 0.0006 | 94.81 | 0.75 | 0.79 | 0.99 | 0.24 | 0.78 | 0.85 | 1.00 | 0.33 |
| c_Ktedonobacteria | 0.0000 | 0.0003 | 0.0105 | 0.0119 | 0.0000 | 312.66 | 0.87 | 0.89 | 0.99 | 0.62 | 0.94 | 0.91 | 1.00 | 0.60 |
| c_Acidobacteriia | 0.0001 | 0.0005 | 0.0354 | 0.0216 | 0.0000 | 415.08 | 0.97 | 0.97 | 1.00 | 0.88 | 1.00 | 0.98 | 1.00 | 0.93 |
| c_DA052 | 0.0000 | 0.0001 | 0.0121 | 0.0137 | 0.0000 | 1040.81 | 0.96 | 0.95 | 0.99 | 0.86 | 0.94 | 0.91 | 0.92 | 0.87 |
| c_Methanomicrobia | 0.0000 | 0.0000 | 0.0113 | 0.0145 | 0.0000 | >100 | 0.93 | 0.94 | 1.00 | 0.79 | 0.99 | 0.97 | 1.00 | 0.87 |
| c_Thaumarchaeota | 0.0000 | 0.0000 | 0.0110 | 0.0168 | 0.0000 | >100 | 0.91 | 0.93 | 1.00 | 0.74 | 0.90 | 0.94 | 1.00 | 0.73 |
| c_MCG | 0.0000 | 0.0000 | 0.0039 | 0.0118 | 0.0000 | >100 | 0.76 | 0.83 | 1.00 | 0.38 | 0.72 | 0.89 | 1.00 | 0.53 |
| c_PRR-12 | 0.0000 | 0.0000 | 0.0026 | 0.0063 | 0.0000 | >100 | 0.72 | 0.81 | 1.00 | 0.29 | 0.63 | 0.86 | 1.00 | 0.40 |
| c_TM1 | 0.0000 | 0.0000 | 0.0018 | 0.0046 | 0.0000 | >100 | 0.69 | 0.81 | 1.00 | 0.29 | 0.62 | 0.85 | 1.00 | 0.33 |
| c_Thermoplasmata | 0.0000 | 0.0000 | 0.0023 | 0.0090 | 0.0014 | >100 | 0.66 | 0.79 | 1.00 | 0.21 | 0.57 | 0.85 | 1.00 | 0.33 |
| c_Chthonomonadetes | 0.0000 | 0.0000 | 0.0018 | 0.0062 | 0.0003 | >100 | 0.65 | 0.77 | 1.00 | 0.14 | 0.55 | 0.82 | 1.00 | 0.20 |

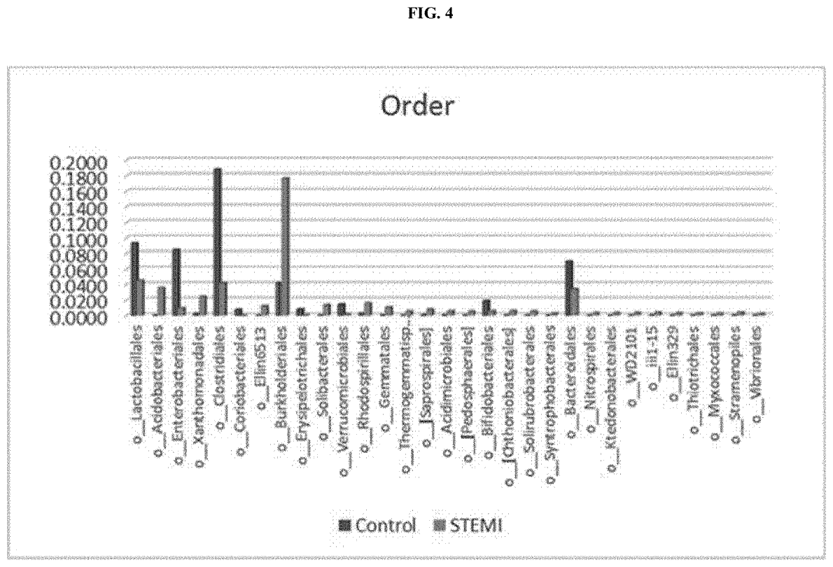

As a result of analyzing bacteria-derived EVs in blood at an order level, a diagnostic model developed using, as a biomarker, one or more bacteria from the order Lactobacillales, the order Acidobacteriales, the order Enterobacteriales, the order Xanthomonadales, the order Clostridiales, the order Coriobacteriales, the order Ellin6513, the order Burkholderiales, the order Erysipelotrichales, the order Solibacterales, the order Verrucomicrobiales, the order Rhodospirillales, the order Gemmatales, the order Thermogemmatisporales, the order Saprospirales, the order Acidimicrobiales, the order Pedosphaerales, the order Bifidobacteriales, the order Chthoniobacterales, the order Solirubrobacterales, the order Syntrophobacterales, the order Bacteroidales, the order Nitrospirales, the order Ktedonobacterales, the order WD2101, the order iii1-15, the order Ellin329, the order Thiotrichales, the order Myxococcales, the order Stramenopiles, and the order Vibrionales exhibited significant diagnostic performance for STEMI (see Table 4 and FIG. 4).

TABLE 4

| | | | | | | | Training Set | | | | Test Set | | | |
|---|---|---|---|---|---|---|---|---|---|---|---|---|---|---|
| | | | | | t-test | | | | Sen- | Spec- | | | Sen- | Spec- |
| | Control | | STEMI | | p- | Ra- | | Acu- | sitiv- | ific- | | Acu- | sitiv- | ific- |
| Taxon | Mean | SD | Mean | SD | value | tio | AUC | rracy | ity | ity | AUC | rracy | ity | ity |
| o_Coriobacteriales | 0.0075 | 0.0091 | 0.0001 | 0.0003 | 0.0000 | 0.01 | 0.90 | 0.82 | 0.83 | 0.77 | 0.80 | 0.79 | 0.73 | 0.94 |
| o_Erysipelotrichales | 0.0082 | 0.0128 | 0.0006 | 0.0021 | 0.0000 | 0.07 | 0.87 | 0.79 | 0.81 | 0.72 | 0.77 | 0.77 | 0.81 | 0.67 |
| o_Enterobacteriales | 0.0848 | 0.0618 | 0.0097 | 0.0154 | 0.0000 | 0.11 | 0.94 | 0.86 | 0.89 | 0.79 | 0.93 | 0.82 | 0.81 | 0.93 |
| o_Verrucomicrobiales | 0.0143 | 0.0181 | 0.0017 | 0.0041 | 0.0000 | 0.12 | 0.85 | 0.77 | 0.84 | 0.54 | 0.78 | 0.80 | 0.81 | 0.78 |
| o_Clostridiales | 0.1882 | 0.1142 | 0.0420 | 0.0315 | 0.0000 | 0.22 | 0.93 | 0.84 | 0.88 | 0.74 | 0.88 | 0.82 | 0.85 | 0.72 |
| o_Bifidobacteriales | 0.0189 | 0.0279 | 0.0057 | 0.0085 | 0.0000 | 0.30 | 0.77 | 0.81 | 0.97 | 0.33 | 0.71 | 0.64 | 0.83 | 0.11 |
| o_Lactobacillales | 0.0933 | 0.0552 | 0.0451 | 0.0329 | 0.0000 | 0.48 | 0.98 | 0.80 | 0.93 | 0.41 | 0.81 | 0.79 | 0.88 | 0.56 |
| o_Bacteroidales | 0.0695 | 0.0488 | 0.0343 | 0.0244 | 0.0000 | 0.49 | 0.75 | 0.73 | 0.91 | 0.21 | 0.75 | 0.74 | 0.96 | 0.17 |
| o_Burkholderiales | 0.0421 | 0.0420 | 0.1764 | 0.1468 | 0.0000 | 4.19 | 0.87 | 0.80 | 0.95 | 0.38 | 0.98 | 0.89 | 1.00 | 0.61 |
| o_Rhodospirillales | 0.0028 | 0.0070 | 0.0159 | 0.0170 | 0.0000 | 5.73 | 0.85 | 0.81 | 0.96 | 0.38 | 0.71 | 0.77 | 0.92 | 0.39 |
| o_Xanthomonadales | 0.0023 | 0.0047 | 0.0244 | 0.0194 | 0.0000 | 10.46 | 0.93 | 0.87 | 0.97 | 0.59 | 0.91 | 0.91 | 0.98 | 0.72 |
| o_Vibrionales | 0.0002 | 0.0009 | 0.0023 | 0.0056 | 0.0070 | 11.05 | 0.60 | 0.78 | 0.99 | 0.15 | 0.50 | 0.76 | 0.98 | 0.17 |
| o_Myxococcales | 0.0002 | 0.0010 | 0.0027 | 0.0052 | 0.0009 | 12.07 | 0.64 | 0.81 | 0.98 | 0.31 | 0.62 | 0.79 | 1.00 | 0.22 |
| o_[Saprospirales] | 0.0006 | 0.0023 | 0.0079 | 0.0166 | 0.0019 | 12.60 | 0.80 | 0.85 | 0.98 | 0.46 | 0.62 | 0.74 | 0.88 | 0.39 |
| o_Solirubrobacterales | 0.0004 | 0.0034 | 0.0055 | 0.0085 | 0.0001 | 13.77 | 0.76 | 0.86 | 1.00 | 0.44 | 0.62 | 0.77 | 0.92 | 0.39 |
| o_Acidimicrobiales | 0.0003 | 0.0013 | 0.0056 | 0.0091 | 0.0000 | 20.03 | 0.79 | 0.87 | 0.98 | 0.54 | 0.48 | 0.74 | 0.94 | 0.22 |
| o_Stramenopiles | 0.0002 | 0.0016 | 0.0041 | 0.0108 | 0.0093 | 22.29 | 0.62 | 0.77 | 0.98 | 0.15 | 0.61 | 0.79 | 1.00 | 0.22 |
| o_iii1-15 | 0.0002 | 0.0014 | 0.0040 | 0.0081 | 0.0009 | 22.71 | 0.68 | 0.79 | 0.99 | 0.21 | 0.57 | 0.77 | 1.00 | 0.17 |
| o_[Chthoniobacterales] | 0.0001 | 0.0009 | 0.0062 | 0.0116 | 0.0003 | 49.89 | 0.77 | 0.84 | 0.98 | 0.41 | 0.62 | 0.80 | 1.00 | 0.28 |
| o_Gemmatales | 0.0002 | 0.0012 | 0.0105 | 0.0130 | 0.0000 | 50.24 | 0.83 | 0.86 | 0.97 | 0.51 | 0.77 | 0.89 | 1.00 | 0.61 |
| o_[Pedosphaerales] | 0.0001 | 0.0006 | 0.0055 | 0.0084 | 0.0000 | 56.31 | 0.79 | 0.85 | 0.97 | 0.49 | 0.72 | 0.80 | 1.00 | 0.28 |
| o_Solibacterales | 0.0002 | 0.0016 | 0.0135 | 0.0155 | 0.0000 | 56.64 | 0.86 | 0.90 | 1.00 | 0.59 | 0.73 | 0.85 | 0.98 | 0.50 |
| o_Nitrospirales | 0.0000 | 0.0004 | 0.0034 | 0.0069 | 0.0006 | 94.81 | 0.74 | 0.83 | 1.00 | 0.33 | 0.57 | 0.76 | 0.98 | 0.17 |
| o_WD2101 | 0.0000 | 0.0004 | 0.0035 | 0.0082 | 0.0028 | 100.30 | 0.69 | 0.81 | 0.99 | 0.28 | 0.50 | 0.76 | 1.00 | 0.11 |
| o_Ellin329 | 0.0000 | 0.0004 | 0.0033 | 0.0087 | 0.0070 | 100.55 | 0.67 | 0.79 | 0.99 | 0.21 | 0.53 | 0.79 | 1.00 | 0.22 |
| o_Ktedonobacterales | 0.0000 | 0.0002 | 0.0034 | 0.0069 | 0.0005 | 174.45 | 0.71 | 0.81 | 0.99 | 0.28 | 0.56 | 0.77 | 1.00 | 0.17 |
| o_Thermogemmatisporales | 0.0000 | 0.0002 | 0.0057 | 0.0082 | 0.0000 | 407.10 | 0.82 | 0.89 | 0.99 | 0.59 | 0.75 | 0.77 | 1.00 | 0.17 |
| o_Acidobacteriales | 0.0001 | 0.0005 | 0.0354 | 0.0216 | 0.0000 | 415.08 | 0.96 | 0.97 | 1.00 | 0.87 | 1.00 | 0.98 | 1.00 | 0.94 |
| o_Thiotrichales | 0.0000 | 0.0000 | 0.0030 | 0.0064 | 0.0008 | 652.57 | 0.67 | 0.79 | 1.00 | 0.18 | 0.55 | 0.80 | 1.00 | 0.28 |
| o_Ellin6513 | 0.0000 | 0.0001 | 0.0121 | 0.0137 | 0.0000 | 1040.81 | 0.88 | 0.93 | 1.00 | 0.72 | 0.76 | 0.89 | 1.00 | 0.61 |
| o_Syntrophobacterales | 0.0000 | 0.0000 | 0.0029 | 0.0066 | 0.0000 | >100 | 0.76 | 0.86 | 1.00 | 0.43 | 0.64 | 0.77 | 1.00 | 0.25 |

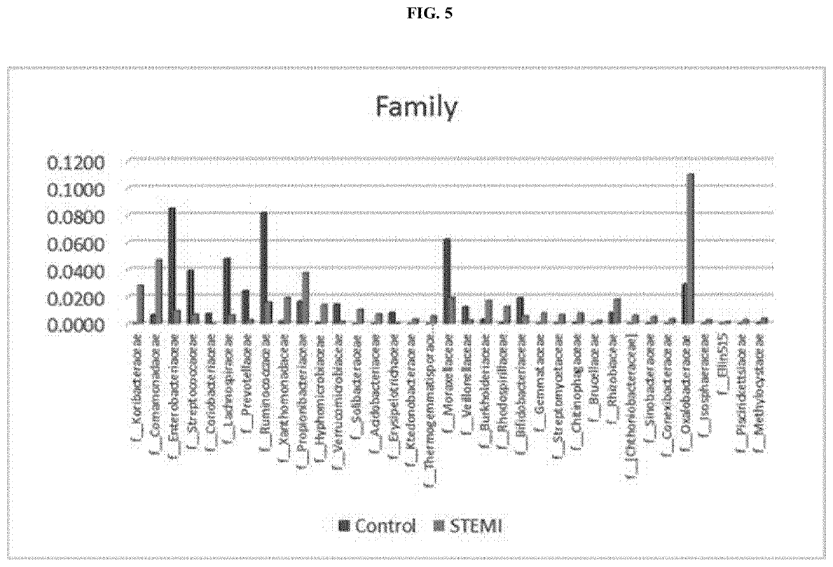

As a result of analyzing bacteria-derived EVs in blood at a family level, a diagnostic model developed using, as a biomarker, one or more bacteria from the family Koribacteraceae, the family Comamonadaceae, the family Enterobacteriaceae, the family Streptococcaceae, the family Coriobacteriaceae, the family Lachnospiraceae, the family Prevotellaceae, the family Ruminococcaceae, the family Xanthomonadaceae, the family Propionibacteriaceae, the family Hyphomicrobiaceae, the family Verrucomicrobiaceae, the family Solibacteraceae, the family Acidobacteriaceae, the family Erysipelotrichaceae, the family Ktedonobacteraceae, the family Thermogemmatisporaceae, the family Moraxellaceae, the family Veillonellaceae, the family Burkholderiaceae, the family Rhodospirillaceae, the family Bifidobacteriaceae, the family Gemmataceae, the family Streptomycetaceae, the family Chitinophagaceae, the family Brucellaceae, the family Rhizobiaceae, the family Chthoniobacteraceae, the family Sinobacteraceae, the family Conexibacteraceae, the family Oxalobacteraceae, the family Isosphaeraceae, the family Ellin515, the family Piscirickettsiaceae, and the family Methylocystaceae exhibited significant diagnostic performance for STEMI (see Table 5 and FIG. 5).

TABLE 5

| | | | | | | | Training Set | | | | Test Set | | | |
|---|---|---|---|---|---|---|---|---|---|---|---|---|---|---|
| | Control | | STEMI | | t-test | | | Ac- | Sensi- | Speci- | | Ac- | Sensi- | Speci- |
| Taxon | Mean | SD | Mean | SD | p-value | Ratio | AUC | urracy | tivity | ficity | AUC | urracy | tivity | ficity |
| f_ | 0.0075 | 0.0091 | 0.0001 | 0.0003 | 0.0000 | 0.01 | 0.90 | 0.81 | 0.81 | 0.82 | 0.88 | 0.76 | 0.74 | 0.85 |
| Coriobacteriaceae | | | | | | | | | | | | | | |
| f_ | 0.0082 | 0.0128 | 0.0006 | 0.0021 | 0.0000 | 0.07 | 0.82 | 0.75 | 0.81 | 0.61 | 0.90 | 0.83 | 0.85 | 0.77 |
| Erysipelotrichaceae | | | | | | | | | | | | | | |
| f_ | 0.0848 | 0.0618 | 0.0097 | 0.0154 | 0.0000 | 0.11 | 0.94 | 0.86 | 0.88 | 0.82 | 0.95 | 0.85 | 0.85 | 0.85 |
| Enterobacteriaceae | | | | | | | | | | | | | | |
| f_ | 0.0143 | 0.0181 | 0.0017 | 0.0041 | 0.0000 | 0.12 | 0.84 | 0.78 | 0.82 | 0.68 | 0.83 | 0.80 | 0.83 | 0.69 |
| Verrucomicrobiaceae | | | | | | | | | | | | | | |
| f_ | 0.0242 | 0.0238 | 0.0029 | 0.0066 | 0.0000 | 0.12 | 0.87 | 0.84 | 0.87 | 0.75 | 0.85 | 0.82 | 0.79 | 0.92 |
| Prevotellaceae | | | | | | | | | | | | | | |
| f_ | 0.0478 | 0.0402 | 0.0064 | 0.0115 | 0.0000 | 0.13 | 0.89 | 0.82 | 0.85 | 0.73 | 0.92 | 0.86 | 0.85 | 0.92 |
| Lachnospiraceae | | | | | | | | | | | | | | |
| f_ | 0.0390 | 0.0289 | 0.0070 | 0.0091 | 0.0000 | 0.18 | 0.91 | 0.84 | 0.88 | 0.73 | 0.88 | 0.85 | 0.92 | 0.54 |
| Streptococcaceae | | | | | | | | | | | | | | |
| f_ | 0.0816 | 0.0599 | 0.0158 | 0.0211 | 0.0000 | 0.19 | 0.87 | 0.81 | 0.85 | 0.68 | 0.90 | 0.83 | 0.85 | 0.77 |
| Ruminococcaccae | | | | | | | | | | | | | | |
| f_ | 0.0124 | 0.0113 | 0.0026 | 0.0047 | 0.0000 | 0.21 | 0.81 | 0.75 | 0.81 | 0.59 | 0.84 | 0.88 | 0.92 | 0.69 |
| Veillonellaceae | | | | | | | | | | | | | | |
| f_ | 0.0189 | 0.0279 | 0.0057 | 0.0085 | 0.0000 | 0.30 | 0.75 | 0.79 | 0.95 | 0.39 | 0.84 | 0.79 | 0.91 | 0.31 |
| Bifidobacteriaceae | | | | | | | | | | | | | | |
| f_ | 0.0621 | 0.0657 | 0.0193 | 0.0331 | 0.0000 | 0.31 | 0.81 | 0.77 | 0.94 | 0.36 | 0.91 | 0.92 | 1.00 | 0.62 |
| Moraxellaceae | | | | | | | | | | | | | | |
| f_ | 0.0081 | 0.0093 | 0.0179 | 0.0134 | 0.0000 | 2.22 | 0.72 | 0.73 | 0.95 | 0.18 | 0.88 | 0.83 | 0.94 | 0.38 |
| Rhizobiaceae | | | | | | | | | | | | | | |
| f_ | 0.0166 | 0.0200 | 0.0374 | 0.0274 | 0.0000 | 2.56 | 0.85 | 0.74 | 0.90 | 0.34 | 0.74 | 0.76 | 0.85 | 0.38 |
| Propionibacteriaceae | | | | | | | | | | | | | | |
| f_ | 0.0291 | 0.0413 | 0.1097 | 0.1497 | 0.0002 | 3.77 | 0.70 | 0.75 | 0.95 | 0.25 | 0.72 | 0.86 | 0.98 | 0.38 |
| Oxalobacteraceae | | | | | | | | | | | | | | |
| f_ | 0.0032 | 0.0087 | 0.0169 | 0.0164 | 0.0000 | 5.26 | 0.80 | 0.77 | 0.94 | 0.36 | 0.85 | 0.8 | 0.98 | 0.23 |
| Burkholderiaceae | | | | | | | | | | | | | | |
| f_ | 0.0006 | 0.0028 | 0.0040 | 0.0083 | 0.0033 | 7.03 | 0.60 | 0.75 | 0.98 | 0.18 | 0.77 | 0.83 | 0.96 | 0.31 |
| Methylocystaceae | | | | | | | | | | | | | | |
| f_ | 0.0067 | 0.0104 | 0.0469 | 0.0378 | 0.0000 | 7.03 | 0.96 | 0.90 | 0.95 | 0.75 | 0.93 | 0.88 | 0.92 | 0.69 |
| Comamonadaceae | | | | | | | | | | | | | | |
| f_ | 0.0019 | 0.0044 | 0.0192 | 0.0182 | 0.0000 | 9.95 | 0.87 | 0.86 | 0.97 | 0.59 | 0.98 | 0.92 | 0.98 | 0.69 |
| Xanthomonadaceae | | | | | | | | | | | | | | |
| f_ | 0.0006 | 0.0023 | 0.0077 | 0.0166 | 0.0023 | 12.59 | 0.73 | 0.84 | 0.99 | 0.45 | 0.67 | 0.79 | 0.89 | 0.38 |
| Chitinophagaceae | | | | | | | | | | | | | | |
| f_ | 0.0004 | 0.0019 | 0.0052 | 0.0078 | 0.0000 | 12.95 | 0.70 | 0.81 | 0.98 | 0.36 | 0.68 | 0.82 | 0.92 | 0.38 |
| Sinobacteraceae | | | | | | | | | | | | | | |
| f_ | 0.0009 | 0.0038 | 0.0127 | 0.0157 | 0.0000 | 13.80 | 0.77 | 0.86 | 0.98 | 0.57 | 0.87 | 0.91 | 0.94 | 0.77 |
| Rhodospirillaceae | | | | | | | | | | | | | | |
| f_ | 0.0008 | 0.0036 | 0.0139 | 0.0150 | 0.0000 | 16.94 | 0.85 | 0.88 | 0.97 | 0.64 | 0.77 | 0.83 | 0.96 | 0.31 |
| Hyphomicrobiaceae | | | | | | | | | | | | | | |
| f_ | 0.0001 | 0.0009 | 0.0024 | 0.0058 | 0.0053 | 20.46 | 0.73 | 0.78 | 0.98 | 0.27 | 0.66 | 0.80 | 0.98 | 0.08 |
| Brucellaceae | | | | | | | | | | | | | | |
| f_ | 0.0001 | 0.0006 | 0.0016 | 0.0033 | 0.0011 | 21.96 | 0.67 | 0.77 | 0.98 | 0.23 | 0.73 | 0.83 | 1.00 | 0.15 |
| Ellin515 | | | | | | | | | | | | | | |
| f_ | 0.0003 | 0.0015 | 0.0066 | 0.0090 | 0.0000 | 23.99 | 0.74 | 0.84 | 0.98 | 0.50 | 0.71 | 0.85 | 0.98 | 0.31 |
| Streptomycetaceae | | | | | | | | | | | | | | |
| f_ | 0.0001 | 0.0010 | 0.0028 | 0.0060 | 0.0015 | 26.15 | 0.68 | 0.78 | 0.99 | 0.25 | 0.71 | 0.82 | 0.98 | 0.15 |
| Isosphaeraceae | | | | | | | | | | | | | | |
| f_ | 0.0001 | 0.0009 | 0.0062 | 0.0116 | 0.0003 | 49.89 | 0.72 | 0.82 | 0.99 | 0.41 | 0.68 | 0.85 | 0.98 | 0.31 |
| [Chthoniobacteraceae] | | | | | | | | | | | | | | |
| f_ | 0.0001 | 0.0007 | 0.0077 | 0.0113 | 0.0000 | 75.29 | 0.75 | 0.85 | 1.00 | 0.48 | 0.76 | 0.85 | 0.96 | 0.38 |
| Gemmataceae | | | | | | | | | | | | | | |
| f_ | 0.0000 | 0.0002 | 0.0036 | 0.0076 | 0.0007 | 150.79 | 0.70 | 0.79 | 0.99 | 0.27 | 0.75 | 0.85 | 1.00 | 0.23 |
| Conexibacteraceae | | | | | | | | | | | | | | |
| f_ | 0.0000 | 0.0002 | 0.0034 | 0.0069 | 0.0005 | 174.45 | 0.81 | 0.85 | 1.00 | 0.48 | 0.77 | 0.85 | 0.98 | 0.31 |
| Ktedonobacteraceae | | | | | | | | | | | | | | |
| f_ | 0.0000 | 0.0003 | 0.0072 | 0.0095 | 0.0000 | 193.78 | 0.82 | 0.85 | 0.99 | 0.50 | 0.73 | 0.88 | 0.98 | 0.46 |
| Acidobacteriaceae | | | | | | | | | | | | | | |
| f_ | 0.0000 | 0.0002 | 0.0057 | 0.0082 | 0.0000 | 407.10 | 0.81 | 0.85 | 1.00 | 0.48 | 0.79 | 0.86 | 0.98 | 0.38 |
| Thermo- | | | | | | | | | | | | | | |
| gemmatisporaceae | | | | | | | | | | | | | | |
| f_ | 0.0000 | 0.0003 | 0.0282 | 0.0177 | 0.0000 | 582.60 | 0.97 | 0.97 | 1.00 | 0.89 | 0.96 | 0.95 | 1.00 | 0.77 |
| Koribacteraceae | | | | | | | | | | | | | | |
| f_ | 0.0000 | 0.0000 | 0.0028 | 0.0062 | 0.0016 | 594.18 | 0.64 | 0.77 | 1.00 | 0.20 | 0.63 | 0.82 | 0.98 | 0.15 |
| Piscirickettsiaceae | | | | | | | | | | | | | | |
| f_ | 0.0000 | 0.0001 | 0.0104 | 0.0122 | 0.0000 | 828.65 | 0.83 | 0.88 | 1.00 | 0.57 | 0.71 | 0.88 | 0.98 | 0.46 |
| Solibacteraceae | | | | | | | | | | | | | | |

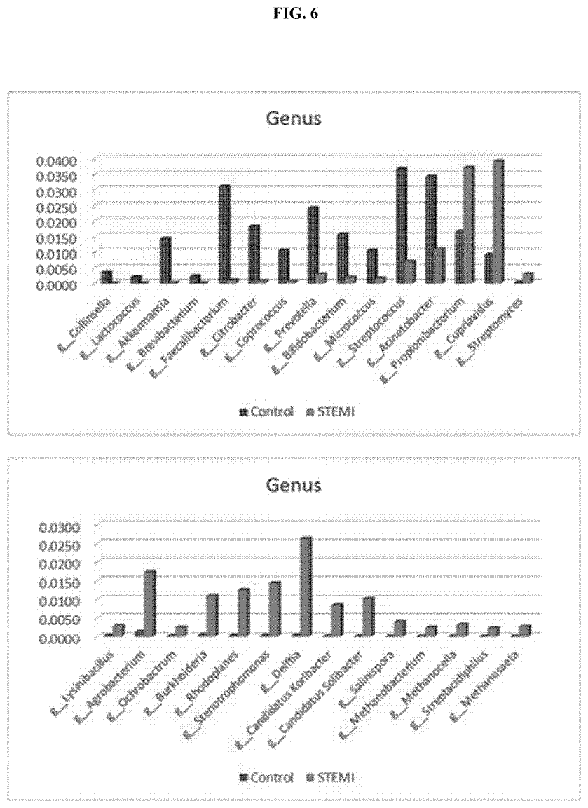

As a result of analyzing bacteria-derived EVs in blood at a genus level, a diagnostic model developed using, as a biomarker, one or more bacteria from the genus Delftia, the genus Agrobacterium, the genus Stenotrophomonas, the genus Faecalibacterium, the genus Candidatus Koribacter, the genus Akkermansia, the genus Streptococcus, the genus Salinispora, the genus Candidatus Solibacter, the genus Citrobacter, the genus Collinsella, the genus Burkholderia, the genus Coprococcus, the genus Rhodoplanes, the genus Acinetobacter, the genus Prevotella, the genus Propionibacterium, the genus Lactococcus, the genus Bifidobacterium, the genus Methanobacterium, the genus Micrococcus, the genus Methanocella, the genus Brevibacterium, the genus Streptacidiphilus, the genus Streptomyces, the genus Ochrobactrum, the genus Methanosaeta, the genus Lysinibacillus, and the genus Cupriavidus exhibited significant diagnostic performance for STEMI (see Table 6 and FIG. 6).

TABLE 6

| | | | | | | | Training Set | | | | Test Set | | | |
|---|---|---|---|---|---|---|---|---|---|---|---|---|---|---|
| | Control | | STEMI | | t-test | | | Ac- | Sensi- | Speci- | | Ac- | Sensi- | Speci- |
| Taxon | Mean | SD | Mean | SD | p-value | Ratio | AUC | urracy | tivity | ficity | AUC | urracy | tivity | ficity |
| g_ | 0.0036 | 0.0062 | 0.0000 | 0.0000 | 0.0000 | 0.00 | 0.85 | 0.75 | 0.74 | 0.79 | 0.85 | 0.76 | 0.76 | 0.73 |
| Collinsella | | | | | | | | | | | | | | |
| g_ | 0.0020 | 0.0038 | 0.0000 | 0.0000 | 0.0001 | 0.00 | 0.82 | 0.76 | 0.94 | 0.29 | 0.82 | 0.80 | 0.96 | 0.27 |
| Lactococcus | | | | | | | | | | | | | | |
| g_ | 0.0143 | 0.0181 | 0.0002 | 0.0013 | 0.0000 | 0.01 | 0.88 | 0.81 | 0.82 | 0.79 | 0.92 | 0.86 | 0.84 | 0.93 |
| Akkermansia | | | | | | | | | | | | | | |
| g_ | 0.0022 | 0.0054 | 0.0000 | 0.0003 | 0.0000 | 0.02 | 0.76 | 0.73 | 0.96 | 0.12 | 0.70 | 0.74 | 0.96 | 0.00 |
| Brevibacterium | | | | | | | | | | | | | | |
| g_ | 0.0311 | 0.0310 | 0.0010 | 0.0037 | 0.0000 | 0.03 | 0.90 | 0.83 | 0.79 | 0.93 | 0.89 | 0.86 | 0.88 | 0.80 |
| Faecalibacterium | | | | | | | | | | | | | | |
| g_ | 0.0182 | 0.0200 | 0.0008 | 0.0024 | 0.0000 | 0.04 | 0.87 | 0.80 | 0.85 | 0.67 | 0.90 | 0.82 | 0.86 | 0.67 |
| Citrobacter | | | | | | | | | | | | | | |
| g_ | 0.0105 | 0.0211 | 0.0007 | 0.0045 | 0.0000 | 0.07 | 0.84 | 0.79 | 0.91 | 0.48 | 0.89 | 0.85 | 0.98 | 0.40 |
| Coprococcus | | | | | | | | | | | | | | |
| g_ | 0.0242 | 0.0238 | 0.0029 | 0.0066 | 0.0000 | 0.12 | 0.83 | 0.77 | 0.85 | 0.57 | 0.97 | 0.92 | 0.94 | 0.87 |
| Prevotella | | | | | | | | | | | | | | |
| g_ | 0.0157 | 0.0266 | 0.0020 | 0.0042 | 0.0000 | 0.13 | 0.81 | 0.81 | 0.90 | 0.57 | 0.91 | 0.86 | 0.88 | 0.80 |
| Bifidobacterium | | | | | | | | | | | | | | |
| g_ | 0.0105 | 0.0165 | 0.0017 | 0.0043 | 0.0000 | 0.16 | 0.79 | 0.77 | 0.97 | 0.21 | 0.69 | 0.80 | 0.98 | 0.2 |
| Micrococcus | | | | | | | | | | | | | | |
| g_ | 0.0368 | 0.0287 | 0.0070 | 0.0091 | 0.0000 | 0.19 | 0.88 | 0.82 | 0.89 | 0.64 | 0.92 | 0.85 | 0.90 | 0.67 |
| Streptococcus | | | | | | | | | | | | | | |
| g_ | 0.0343 | 0.0391 | 0.0109 | 0.0254 | 0.0000 | 0.32 | 0.83 | 0.79 | 0.96 | 0.33 | 0.92 | 0.88 | 0.98 | 0.53 |
| Acinetobacter | | | | | | | | | | | | | | |
| g_ | 0.0165 | 0.0199 | 0.0372 | 0.0274 | 0.0000 | 2.25 | 0.82 | 0.74 | 0.93 | 0.24 | 0.82 | 0.79 | 0.92 | 0.33 |
| Propionibacterium | | | | | | | | | | | | | | |
| g_ | 0.0092 | 0.0145 | 0.0393 | 0.0663 | 0.0014 | 4.25 | 0.62 | 0.77 | 0.96 | 0.26 | 0.54 | 0.79 | 0.96 | 0.20 |
| Cupriavidus | | | | | | | | | | | | | | |
| g_ | 0.0003 | 0.0015 | 0.0029 | 0.0056 | 0.0011 | 10.84 | 0.75 | 0.77 | 0.97 | 0.24 | 0.70 | 0.79 | 1.00 | 0.07 |
| Streptomyces | | | | | | | | | | | | | | |
| g_ | 0.0002 | 0.0013 | 0.0028 | 0.0064 | 0.0035 | 12.28 | 0.68 | 0.79 | 0.99 | 0.24 | 0.66 | 0.80 | 0.98 | 0.20 |
| Lysinibacillus | | | | | | | | | | | | | | |
| g_ | 0.0012 | 0.0035 | 0.0173 | 0.0133 | 0.0000 | 14.01 | 0.97 | 0.94 | 0.96 | 0.86 | 0.74 | 0.86 | 0.94 | 0.60 |
| Agrobacterium | | | | | | | | | | | | | | |
| g_ | 0.0001 | 0.0009 | 0.0024 | 0.0058 | 0.0052 | 21.27 | 0.72 | 0.80 | 0.99 | 0.29 | 0.71 | 0.79 | 0.96 | 0.20 |
| Ochrobactrum | | | | | | | | | | | | | | |
| g_ | 0.0004 | 0.0019 | 0.0109 | 0.0136 | 0.0000 | 27.02 | 0.84 | 0.87 | 0.99 | 0.55 | 0.84 | 0.88 | 0.98 | 0.53 |
| Burkholderia | | | | | | | | | | | | | | |
| g_ | 0.0003 | 0.0024 | 0.0125 | 0.0150 | 0.0000 | 49.85 | 0.83 | 0.89 | 0.99 | 0.62 | 0.84 | 0.86 | 1.00 | 0.40 |
| Rhodoplanes | | | | | | | | | | | | | | |
| g_ | 0.0003 | 0.0008 | 0.0143 | 0.0165 | 0.0000 | 50.88 | 0.92 | 0.91 | 0.98 | 0.71 | 0.96 | 0.92 | 0.94 | 0.87 |
| Stenotrophomonas | | | | | | | | | | | | | | |
| g_Delftia | 0.0003 | 0.0010 | 0.0263 | 0.0202 | 0.0000 | 89.43 | 1.00 | 0.99 | 1.00 | 0.95 | 0.95 | 0.97 | 1.00 | 0.87 |
| g_ | 0.0000 | 0.0002 | 0.0085 | 0.0092 | 0.0000 | 413.93 | 0.90 | 0.90 | 0.99 | 0.67 | 0.74 | 0.85 | 0.98 | 0.40 |
| Candidatus | | | | | | | | | | | | | | |
| Koribacter | | | | | | | | | | | | | | |
| g_ | 0.0000 | 0.0001 | 0.0101 | 0.0120 | 0.0000 | 803.61 | 0.87 | 0.90 | 0.99 | 0.64 | 0.68 | 0.82 | 0.98 | 0.27 |
| Candidatus | | | | | | | | | | | | | | |
| Solibacter | | | | | | | | | | | | | | |
| g_ | 0.0000 | 0.0000 | 0.0038 | 0.0063 | 0.0000 | 10329.07 | 0.87 | 0.92 | 1.00 | 0.71 | 0.87 | 0.91 | 0.98 | 0.67 |
| Salinispora | | | | | | | | | | | | | | |
| g_ | 0.0000 | 0.0000 | 0.0023 | 0.0061 | 0.0000 | >100 | 0.80 | 0.84 | 1.00 | 0.43 | 0.66 | 0.83 | 1.00 | 0.27 |
| Methanobacterium | | | | | | | | | | | | | | |
| g_ | 0.0000 | 0.0000 | 0.0032 | 0.0077 | 0.0000 | >100 | 0.79 | 0.84 | 1.00 | 0.40 | 0.67 | 0.86 | 1.00 | 0.40 |
| Methanocella | | | | | | | | | | | | | | |
| g_ | 0.0000 | 0.0000 | 0.0022 | 0.0055 | 0.0000 | >100 | 0.75 | 0.84 | 1.00 | 0.40 | 0.74 | 0.86 | 1.00 | 0.40 |
| Streptacidiphilus | | | | | | | | | | | | | | |
| g_ | 0.0000 | 0.0000 | 0.0027 | 0.0061 | 0.0000 | >100 | 0.69 | 0.82 | 1.00 | 0.33 | 0.65 | 0.82 | 1.00 | 0.20 |
| Methanosaeta | | | | | | | | | | | | | | |

### Example 5. Dilated Cardiomyopathy Diagnostic Model Based on Meta2enomic Profiling of Bacteria-Derived EVs

EVs were isolated from blood samples of 72 dilated cardiomyopathy (DCMP) patients and 163 normal individuals, the two groups matched in age and gender, and then metagenomic sequencing was performed thereon using the method of Example 3. For the development of a diagnostic model, first, a strain exhibiting a p value of less than 0.05 between two groups in a t-test and a difference of two-fold or more between two groups was selected, and then an AUC, accuracy, sensitivity, and specificity, which are diagnostic performance indexes, were calculated by logistic regression analysis.

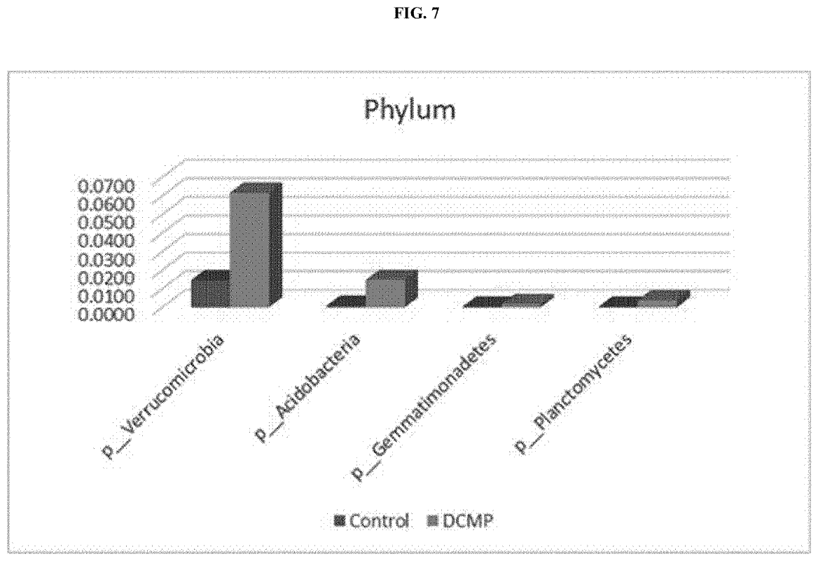

As a result of analyzing bacteria-derived EVs in blood at a phylum level, a diagnostic model developed using, as a biomarker, one or more bacteria from the phylum Verrucomicrobia, the phylum Acidobacteria, the phylum Gemmatimonadetes, and the phylum Planctomycetes exhibited significant diagnostic performance for DCMP (see Table 7 and FIG. 7).

TABLE 7

| | | | | | | | Training Set | | | | Test Set | | | |
|---|---|---|---|---|---|---|---|---|---|---|---|---|---|---|
| | Control | | DCMP | | t-test | | | Ac- | Sensi- | Speci- | | Ac- | Sensi- | Speci- |
| Taxon | Mean | SD | Mean | SD | p-value | Ratio | AUC | urracy | tivity | ficity | AUC | urracy | tivity | ficity |
| p_ | 0.0147 | 0.0183 | 0.0618 | 0.0628 | 0.0000 | 4.20 | 0.80 | 0.81 | 0.97 | 0.46 | 0.70 | 0.82 | 0.98 | 0.40 |
| Verrucomicrobia | | | | | | | | | | | | | | |
| p_ | 0.0004 | 0.0021 | 0.0019 | 0.0030 | 0.0004 | 4.44 | 0.71 | 0.71 | 0.97 | 0.13 | 0.74 | 0.73 | 0.98 | 0.10 |
| Gemmatimonadetes | | | | | | | | | | | | | | |
| p_ | 0.0004 | 0.0017 | 0.0040 | 0.0110 | 0.0079 | 9.88 | 0.64 | 0.71 | 0.96 | 0.17 | 0.65 | 0.75 | 0.98 | 0.15 |
| Planctomycetes | | | | | | | | | | | | | | |
| p_ | 0.0009 | 0.0027 | 0.0148 | 0.0278 | 0.0001 | 15.87 | 0.78 | 0.76 | 0.96 | 0.33 | 0.84 | 0.85 | 0.98 | 0.50 |
| Acidobacteria | | | | | | | | | | | | | | |

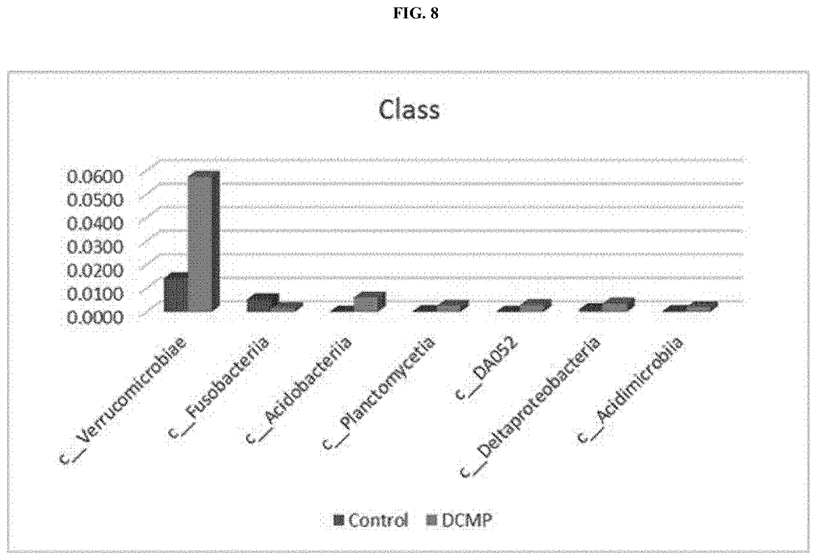

As a result of analyzing bacteria-derived EVs in blood at a class level, a diagnostic model developed using, as a biomarker, one or more bacteria from the class Verrucomicrobiae, the class Fusobacteriia, the class Acidobacteriia, the class Planctomycetia, the class DA052, the class Deltaproteobacteria, and the class Acidimicrobiia exhibited significant diagnostic performance for DCMP (see Table 8 and FIG. 8).

TABLE 8

| | | | | | | | Training Set | | | | Test Set | | | |
|---|---|---|---|---|---|---|---|---|---|---|---|---|---|---|
| | Control | | DCMP | | t-test | | | Ac- | Sensi- | Speci- | | Ac- | Sensi- | Speci- |
| Taxon | Mean | SD | Mean | SD | p-value | Ratio | AUC | urracy | tivity | ficity | AUC | urracy | tivity | ficity |
| c_ | 0.0053 | 0.0085 | 0.0016 | 0.0031 | 0.0000 | 0.30 | 0.70 | 0.66 | 0.95 | 0.08 | 0.60 | 0.70 | 0.92 | 0.11 |
| Fusobacteriia | | | | | | | | | | | | | | |
| c | 0.0011 | 0.0023 | 0.0035 | 0.0074 | 0.0073 | 3.30 | 0.60 | 0.71 | 0.97 | 0.15 | 0.63 | 0.75 | 0.92 | 0.26 |
| Deltaproteobacteria | | | | | | | | | | | | | | |
| c_ | 0.0143 | 0.0181 | 0.0575 | 0.0637 | 0.0000 | 4.01 | 0.71 | 0.79 | 0.98 | 0.40 | 0.67 | 0.85 | 1.00 | 0.42 |
| Verrucomicrobiae | | | | | | | | | | | | | | |
| c_ | 0.0003 | 0.0013 | 0.0019 | 0.0046 | 0.0042 | 6.87 | 0.60 | 0.72 | 0.98 | 0.17 | 0.62 | 0.76 | 0.98 | 0.16 |
| Acidimicrobiia | | | | | | | | | | | | | | |
| c_ | 0.0003 | 0.0015 | 0.0026 | 0.0067 | 0.0064 | 7.82 | 0.63 | 0.68 | 0.96 | 0.09 | 0.62 | 0.76 | 0.98 | 0.16 |
| Planctomycetia | | | | | | | | | | | | | | |
| c_ | 0.0001 | 0.0005 | 0.0061 | 0.0129 | 0.0002 | 71.46 | 0.69 | 0.77 | 0.96 | 0.38 | 0.74 | 0.87 | 1.00 | 0.53 |
| Acidobacteriia | | | | | | | | | | | | | | |
| c_ | 0.0000 | 0.0001 | 0.0029 | 0.0091 | 0.0085 | 251.08 | 0.63 | 0.74 | 0.99 | 0.23 | 0.63 | 0.83 | 1.00 | 0.37 |
| DA052 | | | | | | | | | | | | | | |

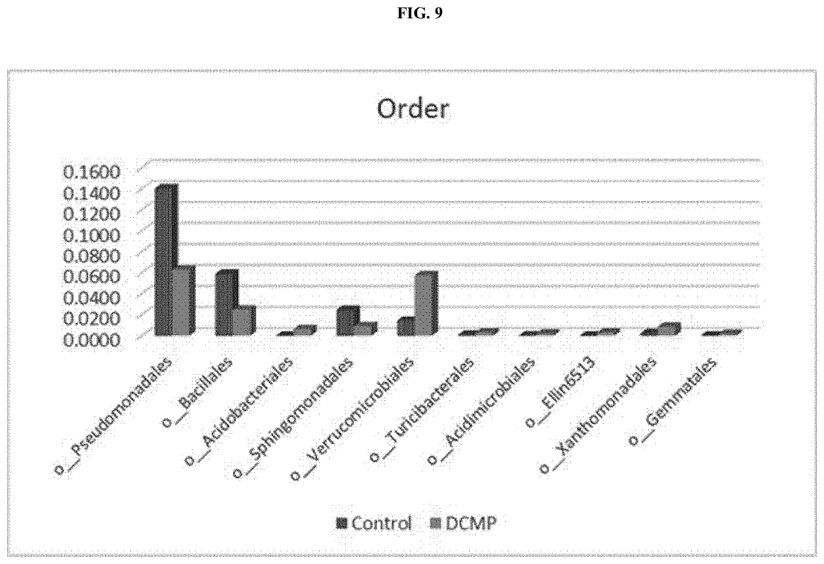

As a result of analyzing bacteria-derived EVs in blood at an order level, a diagnostic model developed using, as a biomarker, one or more bacteria from the order Pseudomonadales, the order Bacillales, the order Acidobacteriales, the order Sphingomonadales, the order Verrucomicrobiales, the order Turicibacterales, the order Acidimicrobiales, the order E11in6513, the order Xanthomonadales, and the order Gemmatales exhibited significant diagnostic performance for DCMP (see Table 9 and FIG. 9).

TABLE 9

| | | | | | t-test | | Training Set | | | | Test Set | | | |
|---|---|---|---|---|---|---|---|---|---|---|---|---|---|---|
| | Control | | DCMP | | p- | | | Ac- | Sensi- | Speci- | | Ac- | Sensi- | Speci- |
| Taxon | Mean | SD | Mean | SD | value | Ratio | AUC | urracy | tivity | ficity | AUC | urracy | tivity | ficity |
| c_ | 0.0053 | 0.0085 | 0.0016 | 0.0031 | 0.0000 | 0.30 | 0.70 | 0.66 | 0.95 | 0.08 | 0.60 | 0.70 | 0.92 | 0.11 |
| Fusobacteriia | | | | | | | | | | | | | | |
| c_ | 0.0011 | 0.0023 | 0.0035 | 0.0074 | 0.0073 | 3.30 | 0.60 | 0.71 | 0.97 | 0.15 | 0.63 | 0.75 | 0.92 | 0.26 |
| Deltaproteobacteria | | | | | | | | | | | | | | |
| c_ | 0.0143 | 0.0181 | 0.0575 | 0.0637 | 0.0000 | 4.01 | 0.71 | 0.79 | 0.98 | 0.40 | 0.67 | 0.85 | 1.00 | 0.42 |
| Verrcomicrobiae | | | | | | | | | | | | | | |
| c_ | 0.0003 | 0.0013 | 0.0019 | 0.0046 | 0.0042 | 6.87 | 0.60 | 0.72 | 0.98 | 0.17 | 0.62 | 0.76 | 0.98 | 0.16 |
| Acidimicrobiia | | | | | | | | | | | | | | |
| c_ | 0.0003 | 0.0015 | 0.0026 | 0.0067 | 0.0064 | 7.82 | 0.63 | 0.68 | 0.96 | 0.09 | 0.62 | 0.76 | 0.98 | 0.16 |
| Planctomycetia | | | | | | | | | | | | | | |
| c_ | 0.0001 | 0.0005 | 0.0061 | 0.0129 | 0.0002 | 71.46 | 0.69 | 0.77 | 0.96 | 0.38 | 0.74 | 0.87 | 1.00 | 0.53 |
| Acidobacteriia | | | | | | | | | | | | | | |
| c_ | 0.0000 | 0.0001 | 0.0029 | 0.0091 | 0.0085 | 251.08 | 0.63 | 0.74 | 0.99 | 0.23 | 0.63 | 0.83 | 1.00 | 0.37 |
| DA052 | | | | | | | | | | | | | | |

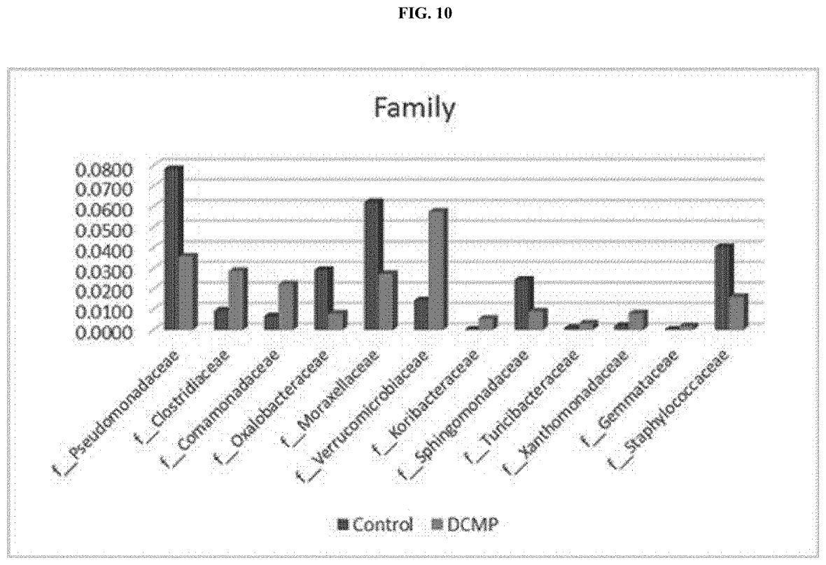

As a result of analyzing bacteria-derived EVs in blood at a family level, a diagnostic model developed using, as a biomarker, one or more bacteria from the family Pseudomonadaceae, the family Clostridiaceae, the family Comamonadaceae, the family Oxalobacteraceae, the family Moraxellaceae, the family Verrucomicrobiaceae, the family Koribacteraceae, the family Sphingomonadaceae, the family Turicibacteraceae, the family Xanthomonadaceae, the family Gemmataceae, and the family Staphylococcaceae exhibited significant diagnostic performance for DCMP (see Table 10 and FIG. 10).

TABLE 10

| | | | | | t-test | | Training Set | | | | Test Set | | | |
|---|---|---|---|---|---|---|---|---|---|---|---|---|---|---|
| | Control | | DCMP | | p- | | | Ac- | Sensi- | Speci- | | Ac- | Sensi- | Speci- |
| Taxon | Mean | SD | Mean | SD | value | Ratio | AUC | urracy | tivity | ficity | AUC | urracy | tivity | ficity |
| f_ | 0.0291 | 0.0413 | 0.0079 | 0.0105 | 0.0000 | 0.27 | 0.75 | 0.70 | 0.91 | 0.18 | 0.67 | 0.63 | 0.85 | 0.17 |
| Oxalobacteraceae | | | | | | | | | | | | | | |
| f_ | 0.0244 | 0.0258 | 0.0089 | 0.0116 | 0.0000 | 0.37 | 0.71 | 0.76 | 1.00 | 0.18 | 0.72 | 0.65 | 0.90 | 0.13 |
| Sphingomonadaceae | | | | | | | | | | | | | | |
| f_ | 0.0403 | 0.0549 | 0.0160 | 0.0134 | 0.0000 | 0.40 | 0.64 | 0.70 | 0.98 | 0.02 | 0.68 | 0.66 | 0.98 | 0.00 |
| Staphylococcaceae | | | | | | | | | | | | | | |
| f_ | 0.0621 | 0.0657 | 0.0272 | 0.0282 | 0.0000 | 0.44 | 0.74 | 0.73 | 0.93 | 0.27 | 0.76 | 0.65 | 0.81 | 0.30 |
| Moraxellaceae | | | | | | | | | | | | | | |
| f_ | 0.0781 | 0.0481 | 0.0356 | 0.0339 | 0.0000 | 0.46 | 0.84 | 0.83 | 0.92 | 0.61 | 0.67 | 0.65 | 0.77 | 0.39 |
| Pseudomonadaceae | | | | | | | | | | | | | | |
| f_ | 0.0093 | 0.0121 | 0.0285 | 0.0212 | 0.0000 | 3.06 | 0.78 | 0.81 | 0.94 | 0.51 | 0.78 | 0.77 | 0.96 | 0.39 |
| Clostridiaceae | | | | | | | | | | | | | | |
| f_ | 0.0009 | 0.0026 | 0.0030 | 0.0060 | 0.0052 | 3.28 | 0.68 | 0.70 | 0.96 | 0.10 | 0.66 | 0.69 | 1.00 | 0.04 |
| Turicibacteraceae | | | | | | | | | | | | | | |
| f_ | 0.0066 | 0.0104 | 0.0221 | 0.0293 | 0.0000 | 3.34 | 0.76 | 0.75 | 0.94 | 0.31 | 0.68 | 0.73 | 0.90 | 0.39 |
| Comamonadaceae | | | | | | | | | | | | | | |
| f_ | 0.0143 | 0.0181 | 0.0575 | 0.0637 | 0.0000 | 4.01 | 0.74 | 0.80 | 0.97 | 0.41 | 0.69 | 0.80 | 0.96 | 0.48 |
| Verrucomicrobiaceae | | | | | | | | | | | | | | |
| f_ | 0.0019 | 0.0044 | 0.0080 | 0.0154 | 0.0017 | 4.13 | 0.67 | 0.73 | 0.97 | 0.16 | 0.54 | 0.70 | 0.98 | 0.13 |
| Xanthomonadaceae | | | | | | | | | | | | | | |
| f_ | 0.0001 | 0.0007 | 0.0017 | 0.0045 | 0.0042 | 16.52 | 0.65 | 0.76 | 0.98 | 0.22 | 0.48 | 0.70 | 1.00 | 0.09 |
| Gemmataceae | | | | | | | | | | | | | | |
| f_ | 0.0000 | 0.0003 | 0.0053 | 0.0122 | 0.0005 | 110.08 | 0.72 | 0.80 | 0.98 | 0.37 | 0.72 | 0.85 | 1.00 | 0.52 |
| Koribacteraceae | | | | | | | | | | | | | | |

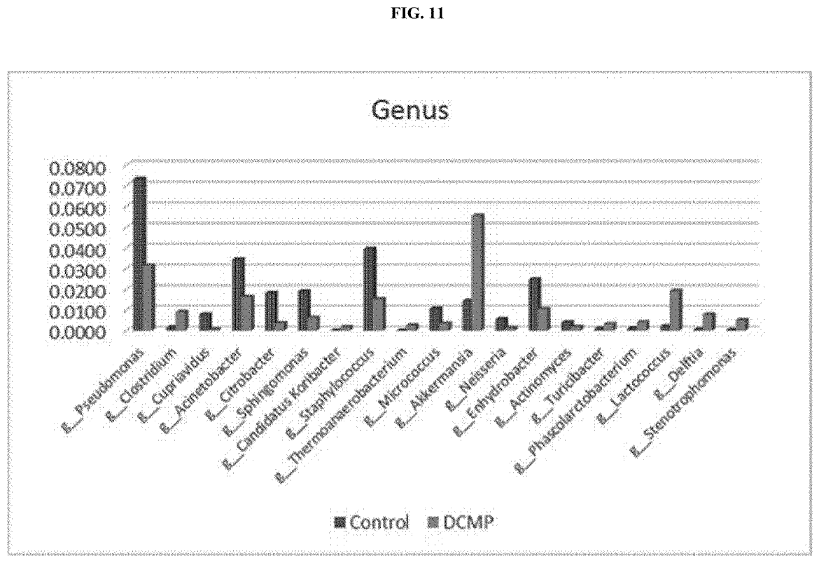

As a result of analyzing bacteria-derived EVs in blood at a genus level, a diagnostic model developed using, as a biomarker, one or more bacteria from the genus Pseudomonas, the genus Clostridium, the genus Cupriavidus, the genus Acinetobacter, the genus Citrobacter, the genus Sphingomonas, the genus Candidatus Koribacter, the genus Staphylococcus, the genus Thermoanaerobacterium, the genus Micrococcus, the genus Akkermansia, the genus Neisseria, the genus Enhydrobacter, the genus Actinomyces, the genus Turicibacter, the genus Phascolarctobacterium, the genus Lactococcus, the genus Delftia, and the genus Stenotrophomonas exhibited significant diagnostic performance for DCMP (see Table 11 and FIG. 11).

TABLE 11

| | | | | | t-test | | Training Set | | | | Test Set | | | |
|---|---|---|---|---|---|---|---|---|---|---|---|---|---|---|
| | Control | | DCMP | | p- | | | Ac- | Sen- | Speci- | | Ac- | Sen- | Speci- |
| Taxon | Mean | SD | Mean | SD | value | Ratio | AUC | urracy | sitivity | ficity | AUC | urracy | sitivity | ficity |
| g_ | 0.0077 | 0.0120 | 0.0006 | 0.0011 | 0.0000 | 0.07 | 0.75 | 0.65 | 0.63 | 0.70 | 0.75 | 0.72 | 0.78 | 0.50 |
| Cupriavidus | | | | | | | | | | | | | | |
| g_ | 0.0181 | 0.0199 | 0.0035 | 0.0045 | 0.0000 | 0.19 | 0.74 | 0.67 | 0.76 | 0.50 | 0.76 | 0.72 | 0.80 | 0.44 |
| Citrobacter | | | | | | | | | | | | | | |
| g_ | 0.0055 | 0.0109 | 0.0012 | 0.0037 | 0.0000 | 0.23 | 0.66 | 0.64 | 0.96 | 0.02 | 0.64 | 0.77 | 0.98 | 0.06 |
| Neisseria | | | | | | | | | | | | | | |
| g_ | 0.0105 | 0.0165 | 0.0033 | 0.0052 | 0.0000 | 0.32 | 0.67 | 0.63 | 0.95 | 0.00 | 0.66 | 0.79 | 1.00 | 0.06 |
| Micrococcus | | | | | | | | | | | | | | |
| g_ | 0.0189 | 0.0220 | 0.0062 | 0.0096 | 0.0000 | 0.33 | 0.73 | 0.68 | 0.88 | 0.29 | 0.73 | 0.73 | 0.87 | 0.25 |
| Sphingomonas | | | | | | | | | | | | | | |
| g_ | 0.0395 | 0.0545 | 0.0152 | 0.0129 | 0.0000 | 0.38 | 0.72 | 0.68 | 0.83 | 0.38 | 0.57 | 0.62 | 0.75 | 0.19 |
| Staphylococcus | | | | | | | | | | | | | | |
| g_ | 0.0246 | 0.0524 | 0.0103 | 0.0142 | 0.0015 | 0.42 | 0.66 | 0.66 | 0.96 | 0.09 | 0.64 | 0.80 | 1.00 | 0.13 |
| Enhydrobacter | | | | | | | | | | | | | | |
| g_ | 0.0733 | 0.0464 | 0.0313 | 0.0323 | 0.0000 | 0.43 | 0.82 | 0.78 | 0.86 | 0.63 | 0.79 | 0.75 | 0.76 | 0.69 |
| Pseudomonas | | | | | | | | | | | | | | |
| g_ | 0.0039 | 0.0060 | 0.0017 | 0.0027 | 0.0002 | 0.44 | 0.66 | 0.64 | 0.96 | 0.02 | 0.56 | 0.80 | 1.00 | 0.13 |
| Actinomyces | | | | | | | | | | | | | | |
| g_ | 0.0343 | 0.0391 | 0.0162 | 0.0208 | 0.0000 | 0.47 | 0.75 | 0.71 | 0.94 | 0.27 | 0.79 | 0.79 | 0.98 | 0.13 |
| Acinetobacter | | | | | | | | | | | | | | |
| g_ | 0.0009 | 0.0026 | 0.0030 | 0.0060 | 0.0052 | 3.28 | 0.66 | 0.69 | 0.93 | 0.23 | 0.62 | 0.79 | 0.89 | 0.44 |
| Turicibacter | | | | | | | | | | | | | | |
| g_ | 0.0011 | 0.0022 | 0.0040 | 0.0064 | 0.0004 | 3.73 | 0.65 | 0.71 | 0.96 | 0.23 | 0.63 | 0.79 | 0.89 | 0.44 |
| Pliaseolarcto- | | | | | | | | | | | | | | |
| bacterium | | | | | | | | | | | | | | |
| g_ | 0.0143 | 0.0181 | 0.0554 | 0.0645 | 0.0000 | 3.88 | 0.67 | 0.77 | 0.97 | 0.38 | 0.68 | 0.87 | 1.00 | 0.44 |
| Akkermansia | | | | | | | | | | | | | | |
| g_ | 0.0015 | 0.0024 | 0.0090 | 0.0110 | 0.0000 | 6.12 | 0.76 | 0.79 | 0.94 | 0.50 | 0.66 | 0.86 | 0.95 | 0.56 |
| Clostridium | | | | | | | | | | | | | | |
| g_ | 0.0020 | 0.0038 | 0.0191 | 0.0509 | 0.0060 | 9.45 | 0.64 | 0.72 | 0.96 | 0.25 | 0.72 | 0.82 | 0.91 | 0.50 |
| Lactococcus | | | | | | | | | | | | | | |
| g_Delftia | 0.0004 | 0.0023 | 0.0077 | 0.0202 | 0.0034 | 18.23 | 0.62 | 0.70 | 0.99 | 0.14 | 0.48 | 0.80 | 1.00 | 0.13 |
| g_ | 0.0003 | 0.0008 | 0.0051 | 0.0136 | 0.0042 | 18.42 | 0.61 | 0.68 | 0.98 | 0.11 | 0.54 | 0.83 | 1.00 | 0.25 |
| Stenotrophomonas | | | | | | | | | | | | | | |
| g_ | 0.0000 | 0.0002 | 0.0016 | 0.0050 | 0.0088 | 78.69 | 0.73 | 0.82 | 1.00 | 0.41 | 0.60 | 0.72 | 0.94 | 0.26 |
| Candidatus | | | | | | | | | | | | | | |
| Koribacter | | | | | | | | | | | | | | |
| g_ | 0.0000 | 0.0000 | 0.0025 | 0.0038 | 0.0000 | >100 | 0.71 | 0.82 | 1.00 | 0.41 | 0.76 | 0.83 | 1.00 | 0.48 |
| Thermo- | | | | | | | | | | | | | | |
| anaerobacterium | | | | | | | | | | | | | | |

### Example 6. Variant Angina Diagnostic Model Based on Metagenomic Profiling of Bacteria-Derived EVs

EVs were isolated from blood samples of 80 variant angina patients and 80 normal individuals, the two groups matched in age and gender, and then metagenomic sequencing was performed thereon using the method of Example 3. For the development of a diagnostic model, first, a strain exhibiting a p value equal to of 0.05 between two groups in a t-test and a difference of two-fold or more between two groups was selected, and then an AUC, accuracy, sensitivity, and specificity, which are diagnostic performance indexes, were calculated by logistic regression analysis.

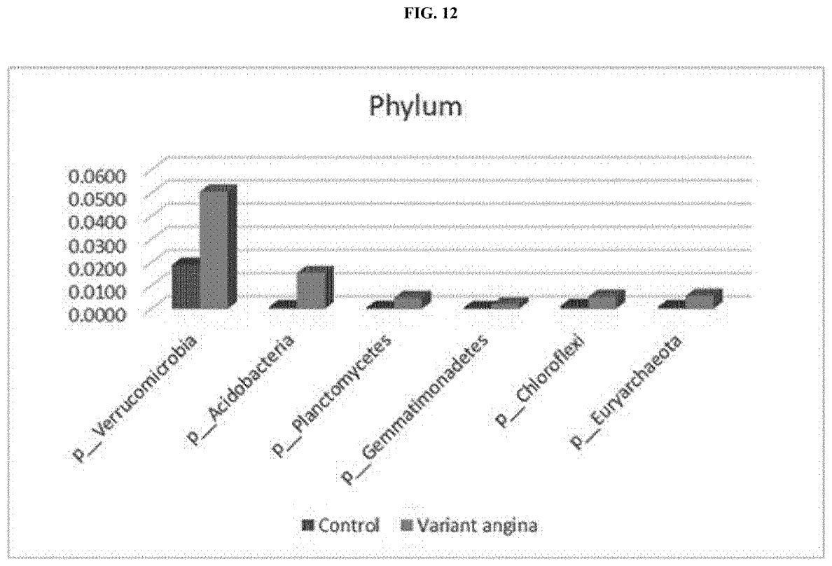

As a result of analyzing bacteria-derived EVs in blood at a phylum level, a diagnostic model developed using, as a biomarker, one or more bacteria from the phylum Verrucomicrobia, the phylum Acidobacteria, the phylum Planctomycetes, the phylum Gemmatimonadetes, the phylum Chloroflexi, and the phylum Euryarchaeota exhibited significant diagnostic performance for variant angina (see Table 12 and FIG. 12).

TABLE 12

| | | | variant | | t-test | | Training Set | | | | Test Set | | | |
|---|---|---|---|---|---|---|---|---|---|---|---|---|---|---|
| | Control | | angina | | p- | | | Ac- | Sen- | Speci- | | Ac- | Sen- | Speci- |
| Taxon | Mean | SD | Mean | SD | value | Ratio | AUC | urracy | sitivity | ficity | AUC | urracy | sitivity | ficity |
| p_ | 0.0190 | 0.0219 | 0.0506 | 0.0550 | 0.0000 | 2.66 | 0.73 | 0.65 | 0.78 | 0.53 | 0.56 | 0.56 | 0.76 | 0.35 |
| Verrucomicrobia | | | | | | | | | | | | | | |
| p_ | 0.0012 | 0.0049 | 0.0052 | 0.0115 | 0.0056 | 4.36 | 0.62 | 0.58 | 0.82 | 0.35 | 0.64 | 0.65 | 0.84 | 0.43 |
| Chloroflexi | | | | | | | | | | | | | | |
| p_ | 0.0003 | 0.0011 | 0.0018 | 0.0034 | 0.0003 | 6.15 | 0.65 | 0.65 | 0.91 | 0.40 | 0.59 | 0.63 | 0.92 | 0.30 |
| Gemmatimonadetes | | | | | | | | | | | | | | |
| p_ | 0.0007 | 0.0017 | 0.0055 | 0.0148 | 0.0049 | 7.98 | 0.54 | 0.54 | 0.71 | 0.37 | 0.60 | 0.52 | 0.60 | 0.43 |
| Euryarchaeota | | | | | | | | | | | | | | |
| p_ | 0.0004 | 0.0018 | 0.0047 | 0.0105 | 0.0005 | 13.28 | 0.68 | 0.63 | 0.87 | 0.39 | 0.69 | 0.71 | 0.92 | 0.48 |
| Planctomycetes | | | | | | | | | | | | | | |
| p_ | 0.0007 | 0.0020 | 0.0154 | 0.0284 | 0.0000 | 21.19 | 0.71 | 0.67 | 0.89 | 0.46 | 0.79 | 0.81 | 0.96 | 0.65 |
| Acidobacteria | | | | | | | | | | | | | | |

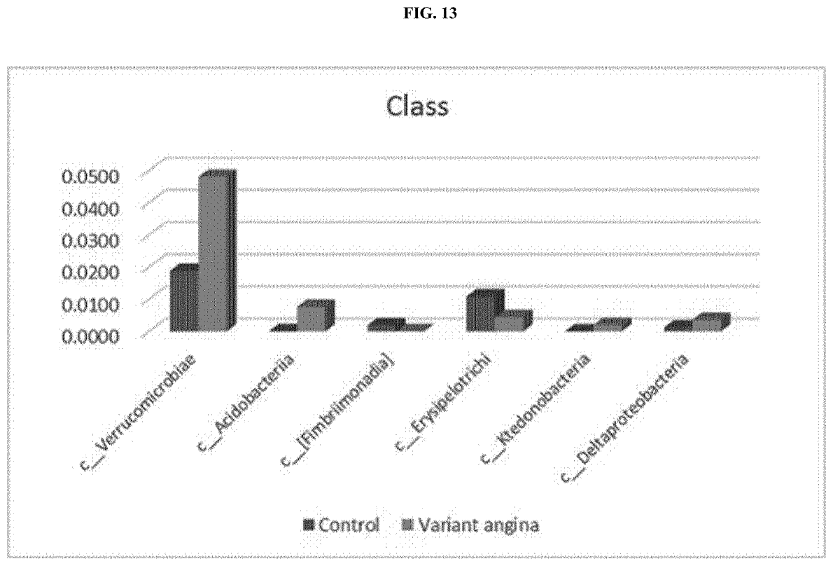

As a result of analyzing bacteria-derived EVs in blood at a class level, a diagnostic model developed using, as a biomarker, one or more bacteria from the class Verrucomicrobiae, the class Acidobacteriia, the class Fimbriimonadia, the class Erysipelotrichi, the class Ktedonobacteria, and the class Deltaproteobacteria exhibited significant diagnostic performance for variant angina (see Table 13 and FIG. 13).

TABLE 13

| | | | | | t- | | | | | | | | | |
|---|---|---|---|---|---|---|---|---|---|---|---|---|---|---|
| | | | variant | | test | | Training Set | | | | Test Set | | | |
| | Control | | angina | | p- | | | Acu- | Sensi- | Speci- | | Acu- | Sensi- | Speci- |
| Taxon | Mean | SD | Mean | SD | value | Ratio | AUC | rracy | tivity | ficity | AUC | rracy | tivity | ficity |
| c_[Fimbriimonadia] | 0.0019 | 0.0054 | 0.0001 | 0.0002 | 0.0030 | 0.03 | 0.65 | 0.62 | 0.27 | 0.96 | 0.62 | 0.63 | 0.38 | 0.88 |
| c_Erysipelotrichi | 0.0109 | 0.0158 | 0.0044 | 0.0062 | 0.0011 | 0.41 | 0.63 | 0.58 | 0.45 | 0.71 | 0.80 | 0.73 | 0.71 | 0.75 |
| c_Verrucomicrobiae | 0.0188 | 0.0218 | 0.0480 | 0.0550 | 0.0000 | 2.55 | 0.68 | 0.63 | 0.80 | 0.46 | 0.53 | 0.56 | 0.71 | 0.42 |
| c_Deltaproteobacteria | 0.0012 | 0.0027 | 0.0036 | 0.0073 | 0.0077 | 2.99 | 0.59 | 0.57 | 0.77 | 0.38 | 0.65 | 0.56 | 0.75 | 0.38 |
| c_Ktedonobacteria | 0.0000 | 0.0002 | 0.0020 | 0.0064 | 0.0073 | 78.53 | 0.61 | 0.56 | 0.93 | 0.20 | 0.63 | 0.65 | 0.96 | 0.33 |
| c_Acidobacteriia | 0.0001 | 0.0004 | 0.0076 | 0.0160 | 0.0001 | 128.78 | 0.67 | 0.63 | 0.95 | 0.32 | 0.76 | 0.75 | 0.92 | 0.58 |

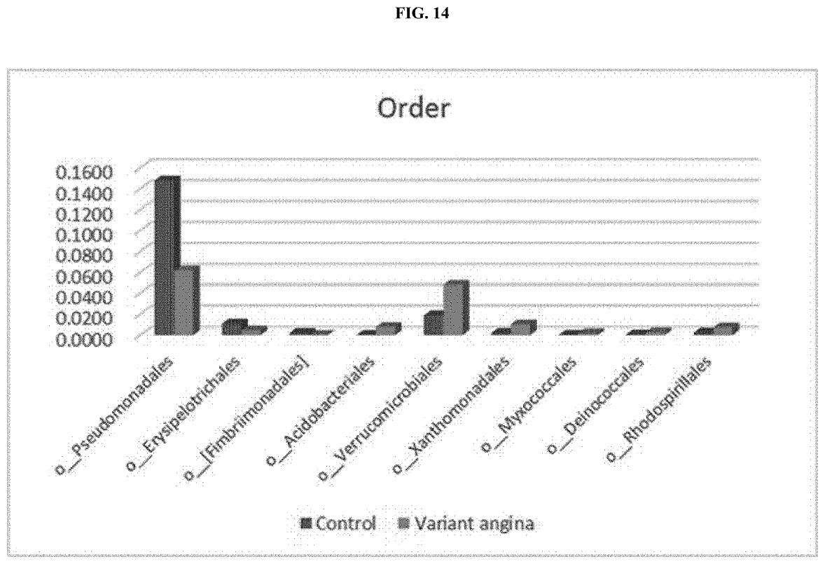

As a result of analyzing bacteria-derived EVs in blood at an order level, a diagnostic model developed using, as a biomarker, one or more bacteria from the order Pseudomonadales, the order Erysipelotrichales, the order Fimbriimonadales, the order Acidobacteriales, the order Verrucomicrobiales, the order Xanthomonadales, the order Myxococcales, the order Deinococcales, and the order Rhodospirillales exhibited significant diagnostic performance for variant angina (see Table 14 and FIG. 14).

TABLE 14

| | | | | | t- | | | | | | | | | |
|---|---|---|---|---|---|---|---|---|---|---|---|---|---|---|
| | | | variant | | test | | Training Set | | | | Test Set | | | |
| | Control | | angina | | p- | | | Acu- | Sensi- | Speci- | | Acu- | Sensi- | Speci- |
| Taxon | Mean | SD | Mean | SD | value | Ratio | AUC | rracy | tivity | ficity | AUC | rracy | tivity | ficity |
| o_[Fimbriimonadales] | 0.0019 | 0.0054 | 0.0001 | 0.0002 | 0.0030 | 0.03 | 0.69 | 0.66 | 0.53 | 0.81 | 0.53 | 0.52 | 0.30 | 0.68 |
| o_Erysipelotrichales | 0.0109 | 0.0158 | 0.0044 | 0.0062 | 0.0011 | 0.41 | 0.70 | 0.66 | 0.65 | 0.67 | 0.56 | 0.63 | 0.45 | 0.75 |
| o_Pseudomonadales | 0.1479 | 0.1176 | 0.0620 | 0.0514 | 0.0000 | 0.42 | 0.83 | 0.74 | 0.72 | 0.77 | 0.62 | 0.56 | 0.50 | 0.61 |
| o_Verrucomicrobiales | 0.0188 | 0.0218 | 0.0480 | 0.0550 | 0.0000 | 2.55 | 0.67 | 0.65 | 0.82 | 0.46 | 0.65 | 0.60 | 0.95 | 0.36 |
| o_Rhodospirillales | 0.0023 | 0.0053 | 0.0071 | 0.0142 | 0.0056 | 3.07 | 0.59 | 0.60 | 0.85 | 0.31 | 0.63 | 0.52 | 0.80 | 0.32 |
| o_Deinococcales | 0.0006 | 0.0016 | 0.0025 | 0.0061 | 0.0085 | 4.32 | 0.62 | 0.60 | 0.82 | 0.35 | 0.51 | 0.44 | 0.75 | 0.25 |
| o_Xanthomonadales | 0.0021 | 0.0048 | 0.0101 | 0.0208 | 0.0011 | 4.94 | 0.65 | 0.61 | 0.90 | 0.27 | 0.68 | 0.60 | 0.95 | 0.36 |
| o_Myxococcales | 0.0001 | 0.0003 | 0.0017 | 0.0037 | 0.0002 | 20.19 | 0.64 | 0.67 | 0.93 | 0.37 | 0.59 | 0.58 | 0.95 | 0.32 |
| o_Acidobacteriales | 0.0001 | 0.0004 | 0.0076 | 0.0160 | 0.0001 | 128.78 | 0.68 | 0.71 | 0.98 | 0.40 | 0.63 | 0.60 | 0.95 | 0.36 |

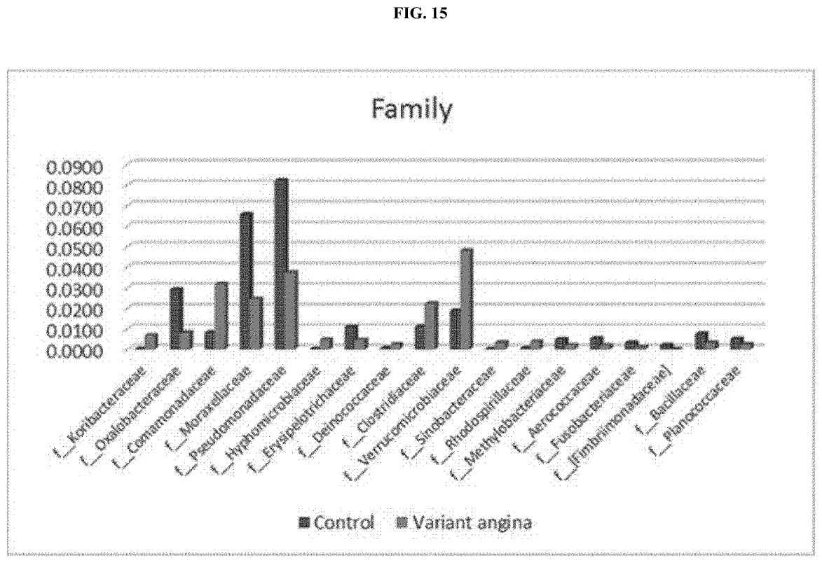

As a result of analyzing bacteria-derived EVs in blood at a family level, a diagnostic model developed using, as a biomarker, one or more bacteria from the family Koribacteraceae, the family Oxalobacteraceae, the family Comamonadaceae, the family Moraxellaceae, the family Pseudomonadaceae, the family Hyphomicrobiaceae, the family Erysipelotrichaceae, the family Deinococcaceae, the family Clostridiaceae, the family Verrucomicrobiaceae, the family Sinobacteraceae, the family Rhodospirillaceae, the family Methylobacteriaceae, the family Aerococcaceae, the family Fusobacteriaceae, the family Fimbriimonadaceae, the family Bacillaceae, and the family Planococcaceae exhibited significant diagnostic performance for variant angina (see Table 15 and FIG. 15).

TABLE 15

| | | | | | t- | | | | | | | | | |
|---|---|---|---|---|---|---|---|---|---|---|---|---|---|---|
| | | | variant | | test | | Training Set | | | | Test Set | | | |
| | Control | | angina | | p- | | | Acu- | Sensi- | Speci- | | Acu- | Sensi- | Speci- |
| Taxon | Mean | SD | Mean | SD | value | Ratio | AUC | rracy | tivity | ficity | AUC | rracy | tivity | ficity |
| f_[Fimbriimonadaceae] | 0.0019 | 0.0054 | 0.0001 | 0.0002 | 0.0030 | 0.03 | 0.62 | 0.59 | 0.32 | 0.86 | 0.67 | 0.67 | 0.50 | 0.83 |
| f_Oxalobacteraceae | 0.0291 | 0.0505 | 0.0081 | 0.0103 | 0.0005 | 0.28 | 0.73 | 0.66 | 0.57 | 0.75 | 0.63 | 0.58 | 0.38 | 0.79 |
| f_Fusobacteriaceae | 0.0032 | 0.0058 | 0.0010 | 0.0019 | 0.0019 | 0.32 | 0.62 | 0.55 | 0.45 | 0.66 | 0.60 | 0.58 | 0.46 | 0.71 |
| f_Aerococcaceae | 0.0052 | 0.0101 | 0.0018 | 0.0033 | 0.0054 | 0.35 | 0.63 | 0.62 | 0.41 | 0.82 | 0.55 | 0.58 | 0.42 | 0.75 |
| f_Moraxellaceae | 0.0657 | 0.0776 | 0.0245 | 0.0334 | 0.0000 | 0.37 | 0.72 | 0.70 | 0.59 | 0.80 | 0.81 | 0.71 | 0.58 | 0.83 |
| f_Methylobacteriaceae | 0.0048 | 0.0062 | 0.0020 | 0.0048 | 0.0014 | 0.41 | 0.66 | 0.62 | 0.43 | 0.80 | 0.71 | 0.69 | 0.58 | 0.79 |
| f_Erysipelotrichaceae | 0.0109 | 0.0158 | 0.0044 | 0.0062 | 0.0011 | 0.41 | 0.70 | 0.66 | 0.57 | 0.75 | 0.61 | 0.63 | 0.63 | 0.63 |
| f_Bacillaceae | 0.0075 | 0.0107 | 0.0032 | 0.0043 | 0.0014 | 0.43 | 0.61 | 0.60 | 0.45 | 0.75 | 0.64 | 0.54 | 0.54 | 0.54 |
| f_Pseudomonadaceae | 0.0822 | 0.0753 | 0.0374 | 0.0319 | 0.0000 | 0.46 | 0.70 | 0.70 | 0.61 | 0.79 | 0.74 | 0.67 | 0.58 | 0.75 |
| f_Planococcaceae | 0.0050 | 0.0074 | 0.0024 | 0.0035 | 0.0057 | 0.48 | 0.60 | 0.57 | 0.45 | 0.70 | 0.69 | 0.69 | 0.54 | 0.83 |
| f_Clostridiaceae | 0.0110 | 0.0163 | 0.0223 | 0.0230 | 0.0005 | 2.03 | 0.68 | 0.66 | 0.73 | 0.59 | 0.60 | 0.58 | 0.71 | 0.46 |
| f_Verrucomicrobiaceae | 0.0188 | 0.0218 | 0.0480 | 0.0550 | 0.0000 | 2.55 | 0.67 | 0.62 | 0.79 | 0.45 | 0.58 | 0.58 | 0.71 | 0.46 |
| f_Comamonadaceae | 0.0081 | 0.0109 | 0.0317 | 0.0529 | 0.0002 | 3.93 | 0.73 | 0.69 | 0.79 | 0.59 | 0.63 | 0.54 | 0.63 | 0.46 |
| f_Deinococcaceae | 0.0005 | 0.0014 | 0.0023 | 0.0060 | 0.0088 | 4.89 | 0.69 | 0.63 | 0.88 | 0.38 | 0.42 | 0.46 | 0.67 | 0.25 |
| f_Rhodospirillacea | 0.0005 | 0.0017 | 0.0036 | 0.0077 | 0.0006 | 7.62 | 0.67 | 0.57 | 0.75 | 0.39 | 0.47 | 0.54 | 0.75 | 0.33 |
| f_Hyphomicrobiaceae | 0.0001 | 0.0005 | 0.0046 | 0.0122 | 0.0016 | 38.87 | 0.70 | 0.63 | 0.89 | 0.36 | 0.46 | 0.52 | 0.79 | 0.25 |
| f_Sinobacteraceae | 0.0001 | 0.0003 | 0.0033 | 0.0095 | 0.0033 | 58.87 | 0.67 | 0.62 | 0.84 | 0.39 | 0.57 | 0.60 | 0.88 | 0.33 |
| f_Koribacteraceae | 0.0001 | 0.0004 | 0.0067 | 0.0142 | 0.0001 | 118.33 | 0.75 | 0.67 | 0.91 | 0.43 | 0.53 | 0.60 | 0.92 | 0.29 |

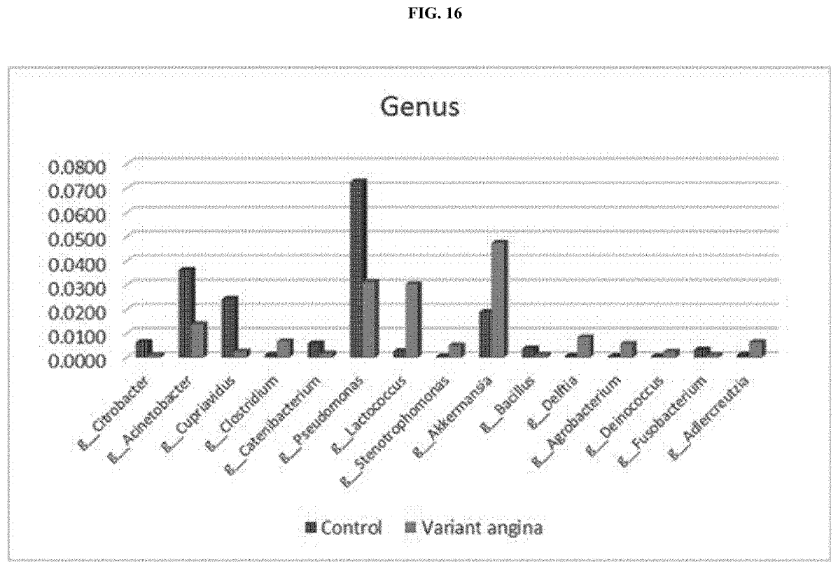

As a result of analyzing bacteria-derived EVs in blood at a genus level, a diagnostic model developed using, as a biomarker, one or more bacteria from the genus Citrobacter, the genus Acinetobacter, the genus Cupriavidus, the genus Clostridium, the genus Catenibacterium, the genus Pseudomonas, the genus Lactococcus, the genus Stenotrophomonas, the genus Akkermansia, the genus Bacillus, the genus Delftia, the genus Agrobacterium, the genus Deinococcus, the genus Fusobacterium, and the genus Adlercreutzia exhibited significant diagnostic performance for variant angina (see Table 16 and FIG. 16).

TABLE 16

| | | | | | t- | | | | | | | | | |
|---|---|---|---|---|---|---|---|---|---|---|---|---|---|---|
| | | | variant | | test | | Training Set | | | | Test Set | | | |
| | Control | | angina | | p- | | | Acu- | Sensi- | Speci- | | Acu- | Sensi- | Speci- |
| Taxon | Mean | SD | Mean | SD | value | Ratio | AUC | rracy | tivity | ficity | AUC | rracy | tivity | ficity |
| g_Citrobacter | 0.0063 | 0.0080 | 0.0009 | 0.0018 | 0.0000 | 0.15 | 0.81 | 0.71 | 0.54 | 0.91 | 0.69 | 0.65 | 0.57 | 0.72 |
| g_Acinetobacter | 0.0362 | 0.0522 | 0.0138 | 0.0252 | 0.0008 | 0.38 | 0.77 | 0.73 | 0.67 | 0.80 | 0.74 | 0.73 | 0.70 | 0.76 |
| g_Cupriavidus | 0.0242 | 0.0492 | 0.0024 | 0.0040 | 0.0002 | 0.10 | 0.74 | 0.68 | 0.56 | 0.80 | 0.86 | 0.81 | 0.61 | 1.00 |
| g_Clostridium | 0.0011 | 0.0024 | 0.0066 | 0.0087 | 0.0000 | 6.11 | 0.73 | 0.73 | 0.95 | 0.51 | 0.60 | 0.65 | 0.87 | 0.44 |
| g_Catenibacterium | 0.0058 | 0.0122 | 0.0016 | 0.0039 | 0.0046 | 0.28 | 0.72 | 0.67 | 0.54 | 0.80 | 0.69 | 0.73 | 0.65 | 0.80 |
| g_Pseudomonas | 0.0727 | 0.0649 | 0.0313 | 0.0308 | 0.0000 | 0.43 | 0.71 | 0.71 | 0.68 | 0.75 | 0.79 | 0.75 | 0.65 | 0.84 |
| g_Lactococcus | 0.0026 | 0.0048 | 0.0303 | 0.0710 | 0.0009 | 11.64 | 0.69 | 0.62 | 0.82 | 0.40 | 0.46 | 0.54 | 0.83 | 0.28 |
| g_Stenotrophomonas | 0.0005 | 0.0021 | 0.0050 | 0.0136 | 0.0051 | 10.01 | 0.66 | 0.64 | 0.93 | 0.35 | 0.50 | 0.54 | 0.91 | 0.20 |
| g_Akkermansia | 0.0188 | 0.0219 | 0.0474 | 0.0548 | 0.0000 | 2.53 | 0.66 | 0.65 | 0.77 | 0.53 | 0.59 | 0.60 | 0.74 | 0.48 |
| g_Bacillus | 0.0037 | 0.0074 | 0.0011 | 0.0026 | 0.0038 | 0.29 | 0.65 | 0.62 | 0.47 | 0.76 | 0.58 | 0.63 | 0.43 | 0.80 |
| g_Delftia | 0.0007 | 0.0035 | 0.0082 | 0.0224 | 0.0039 | 12.30 | 0.64 | 0.59 | 0.82 | 0.35 | 0.46 | 0.46 | 0.74 | 0.20 |
| g_Agrobacterium | 0.0005 | 0.0014 | 0.0056 | 0.0131 | 0.0010 | 10.73 | 0.62 | 0.54 | 0.84 | 0.24 | 0.58 | 0.58 | 0.91 | 0.28 |
| g_Deinococcus | 0.0005 | 0.0014 | 0.0023 | 0.0060 | 0.0088 | 4.89 | 0.60 | 0.59 | 0.86 | 0.31 | 0.57 | 0.60 | 0.87 | 0.36 |
| g_Fusobacterium | 0.0032 | 0.0058 | 0.0010 | 0.0019 | 0.0017 | 0.31 | 0.59 | 0.53 | 0.35 | 0.71 | 0.60 | 0.58 | 0.43 | 0.72 |
| g_Adlercreutzia | 0.0011 | 0.0026 | 0.0063 | 0.0147 | 0.0026 | 5.62 | 0.58 | 0.52 | 0.67 | 0.36 | 0.55 | 0.52 | 0.70 | 0.36 |

### Example 7. Atrial Fibrillation Diagnostic Model Based on Metagenomic Profiling of Bacteria-Derived EVs

EVs were isolated from blood samples of 34 atrial fibrillation patients and 62 normal individuals, the two groups matched in age and gender, and then metagenomic sequencing was performed thereon using the method of Example 3. For the development of a diagnostic model, first, a strain exhibiting a p value of less than 0.05 between two groups in a t-test and a difference of two-fold or more between two groups was selected, and then an AUC, accuracy, sensitivity, and specificity, which are diagnostic performance indexes, were calculated by logistic regression analysis.

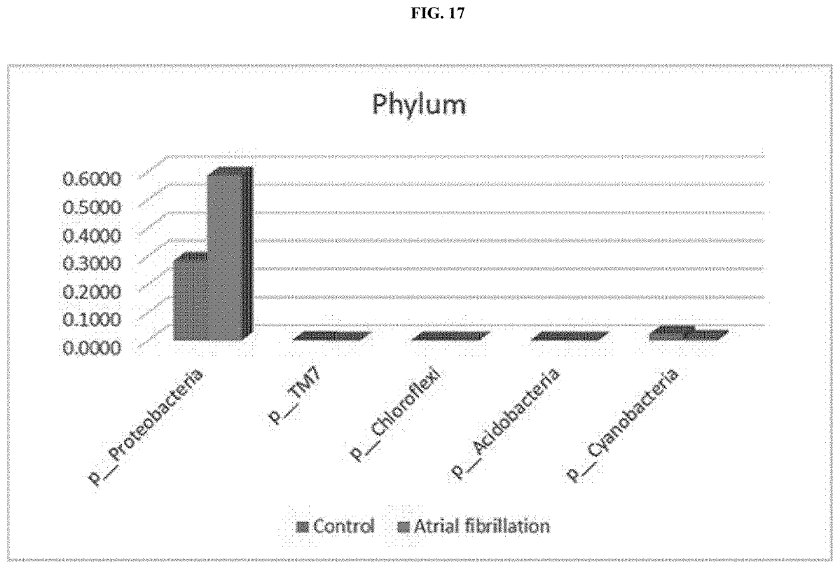

As a result of analyzing bacteria-derived EVs in blood at a phylum level, a diagnostic model developed using, as a biomarker, one or more bacteria from the phylum Proteobacteria, the phylum TM7, the phylum Chloroflexi, the phylum Acidobacteria, and the phylum Cyanobacteria exhibited significant diagnostic performance for atrial fibrillation (see Table 17 and FIG. 17).

TABLE 17

| | | | | | t- | | | | | | | | | |
|---|---|---|---|---|---|---|---|---|---|---|---|---|---|---|
| | | | Atrial | | test | | Training Set | | | | Test Set | | | |
| | Control | | fibrillation | | p- | | | Acu- | Sensi- | Speci- | | Acu- | Sensi- | Speci- |
| Taxon | Mean | SD | Mean | SD | value | Ratio | AUC | rracy | tivity | ficity | AUC | rracy | tivity | ficity |
| p_Chloroflexi | 0.0007 | 0.0017 | 0.0000 | 0.0002 | 0.0032 | 0.06 | 0.66 | 0.60 | 0.95 | 0.04 | 0.63 | 0.76 | 0.91 | 0.17 |
| p_Acidobacteria | 0.0008 | 0.0027 | 0.0001 | 0.0003 | 0.0346 | 0.07 | 0.66 | 0.60 | 0.90 | 0.12 | 0.59 | 0.72 | 0.83 | 0.33 |
| p_TM7 | 0.0021 | 0.0043 | 0.0002 | 0.0008 | 0.0010 | 0.09 | 0.70 | 0.67 | 0.85 | 0.38 | 0.56 | 0.62 | 0.74 | 0.17 |
| p_Cyanobacteria | 0.0225 | 0.0398 | 0.0063 | 0.0067 | 0.0025 | 0.28 | 0.64 | 0.64 | 0.85 | 0.31 | 0.56 | 0.72 | 0.87 | 0.17 |
| p_Proteobacteria | 0.2833 | 0.1040 | 0.5866 | 0.2046 | 0.0000 | 2.07 | 0.86 | 0.88 | 0.95 | 0.77 | 0.85 | 0.97 | 1.00 | 0.83 |

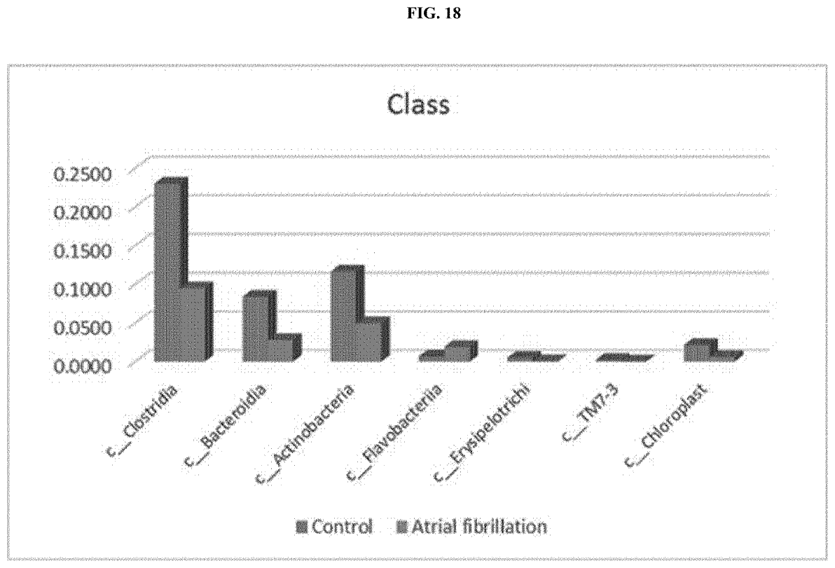

As a result of analyzing bacteria-derived EVs in blood at a class level, a diagnostic model developed using, as a biomarker, one or more bacteria from the class Clostridia, the class Bacteroidia, the class Actinobacteria, the class Flavobacteriia, the class Erysipelotrichi, the class TM7-3, and the class Chloroplast exhibited significant diagnostic performance for atrial fibrillation (see Table 18 and FIG. 18).

TABLE 18

| | | | | | t- | | | | | | | | | |
|---|---|---|---|---|---|---|---|---|---|---|---|---|---|---|
| | | | Atrial | | test | | Training Set | | | | Test Set | | | |
| | Control | | fibrillation | | p- | | | Acu- | Sensi- | Speci- | | Acu- | Sensi- | Speci- |
| Taxon | Mean | SD | Mean | SD | value | Ratio | AUC | rracy | tivity | ficity | AUC | rracy | tivity | ficity |
| c_TM | 0.0020 | 0.0043 | 0.0002 | 0.0008 | 0.0014 | 0.09 | 0.68 | 0.70 | 1.00 | 0.05 | 0.56 | 0.57 | 0.83 | 0.18 |
| 7-3 | | | | | | | | | | | | | | |
| c_Erysipelotrichi | 0.0048 | 0.0073 | 0.0004 | 0.0007 | 0.0000 | 0.09 | 0.78 | 0.72 | 0.80 | 0.52 | 0.55 | 0.48 | 0.67 | 0.18 |
| c_Chloroplast | 0.0215 | 0.0400 | 0.0059 | 0.0068 | 0.0037 | 0.27 | 0.65 | 0.67 | 0.93 | 0.10 | 0.44 | 0.52 | 0.83 | 0.00 |
| c_Bacteroidia | 0.0838 | 0.0466 | 0.0278 | 0.0225 | 0.0000 | 0.33 | 0.85 | 0.76 | 0.83 | 0.62 | 0.89 | 0.86 | 0.89 | 0.82 |
| c_Clostridia | 0.2298 | 0.1146 | 0.0946 | 0.0589 | 0.0000 | 0.41 | 0.86 | 0.84 | 0.91 | 0.67 | 0.88 | 0.79 | 0.89 | 0.64 |
| c_Actinobacteria | 0.1164 | 0.0741 | 0.0496 | 0.0426 | 0.0000 | 0.43 | 0.79 | 0.78 | 0.87 | 0.57 | 0.84 | 0.83 | 0.94 | 0.64 |
| c_Flavobacteriia | 0.0068 | 0.0159 | 0.0188 | 0.0125 | 0.0003 | 2.78 | 0.79 | 0.75 | 0.96 | 0.29 | 0.80 | 0.69 | 0.89 | 0.36 |

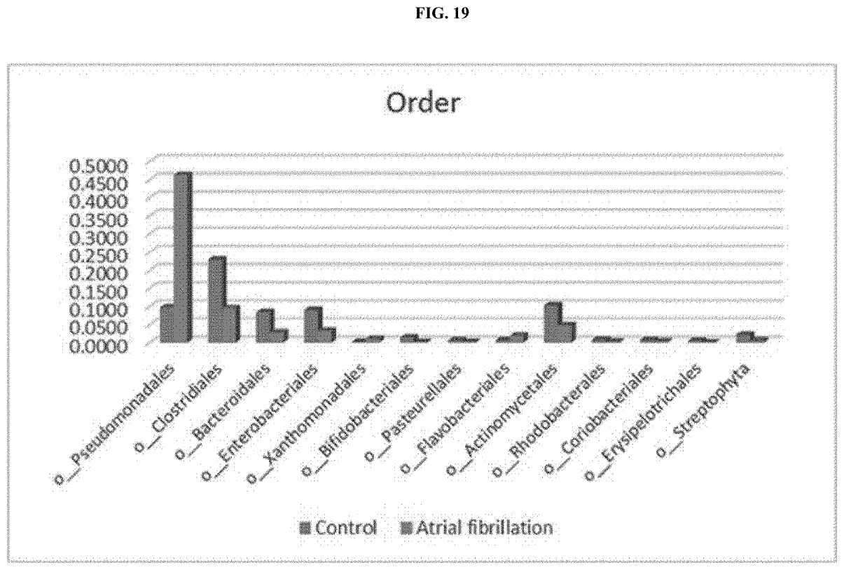

As a result of analyzing bacteria-derived EVs in blood at an order level, a diagnostic model developed using, as a biomarker, one or more bacteria from the order Pseudomonadales, the order Clostridiales, the order Bacteroidales, the order Enterobacteriales, the order Xanthomonadales, the order Bifidobacteriales, the order Pasteurellales, the order Flavobacteriales, the order Actinomycetales, the order Rhodobacterales, the order Coriobacteriales, the order Erysipelotrichales, and the order Streptophyta exhibited significant diagnostic performance for atrial fibrillation (see Table 19 and FIG. 19).

TABLE 19

| | | | | | t- | | | | | | | | | |
|---|---|---|---|---|---|---|---|---|---|---|---|---|---|---|
| | | | Atrial | | test | | Training Set | | | | Test Set | | | |
| | Control | | fibrillation | | p- | | | Acu- | Sensi- | Speci- | | Acu- | Sensi- | Speci- |
| Taxon | Mean | SD | Mean | SD | value | Ratio | AUC | rracy | tivity | ficity | AUC | rracy | tivity | ficity |
| o_Erysipelotrichales | 0.0048 | 0.0073 | 0.0004 | 0.0007 | 0.0000 | 0.09 | 0.70 | 0.63 | 0.70 | 0.48 | 0.81 | 0.72 | 0.85 | 0.44 |
| o_Bifidobacteriales | 0.0136 | 0.0154 | 0.0018 | 0.0020 | 0.0000 | 0.13 | 0.83 | 0.73 | 0.77 | 0.65 | 0.72 | 0.66 | 0.75 | 0.44 |
| o_Streptophyta | 0.0214 | 0.0401 | 0.0059 | 0.0068 | 0.0038 | 0.28 | 0.64 | 0.67 | 0.91 | 0.22 | 0.51 | 0.59 | 0.70 | 0.33 |
| o_Rhodobacterales | 0.0081 | 0.0144 | 0.0026 | 0.0045 | 0.0071 | 0.32 | 0.73 | 0.69 | 0.89 | 0.30 | 0.53 | 0.59 | 0.65 | 0.44 |
| o_Pasteurellales | 0.0061 | 0.0087 | 0.0020 | 0.0037 | 0.0021 | 0.33 | 0.77 | 0.69 | 0.89 | 0.30 | 0.56 | 0.62 | 0.70 | 0.44 |
| o_Bacteroidales | 0.0838 | 0.0466 | 0.0278 | 0.0225 | 0.0000 | 0.33 | 0.89 | 0.78 | 0.82 | 0.70 | 0.84 | 0.79 | 0.85 | 0.67 |
| o_Enterobacteriales | 0.0902 | 0.0565 | 0.0326 | 0.0390 | 0.0000 | 0.36 | 0.86 | 0.78 | 0.80 | 0.74 | 0.83 | 0.72 | 0.65 | 0.89 |
| o_Clostridiales | 0.2294 | 0.1144 | 0.0946 | 0.0589 | 0.0000 | 0.41 | 0.89 | 0.81 | 0.86 | 0.70 | 0.81 | 0.76 | 0.80 | 0.67 |
| o_Coriobacteriales | 0.0070 | 0.0087 | 0.0031 | 0.0061 | 0.0154 | 0.45 | 0.71 | 0.61 | 0.91 | 0.04 | 0.50 | 0.59 | 0.80 | 0.11 |
| o_Actinomycetales | 0.1028 | 0.0748 | 0.0479 | 0.0429 | 0.0000 | 0.47 | 0.74 | 0.70 | 0.84 | 0.43 | 0.75 | 0.62 | 0.65 | 0.56 |
| o_Flavobacteriales | 0.0068 | 0.0159 | 0.0188 | 0.0125 | 0.0003 | 2.78 | 0.76 | 0.84 | 0.91 | 0.70 | 0.84 | 0.83 | 0.85 | 0.78 |
| o_Xanthomonadales | 0.0023 | 0.0043 | 0.0103 | 0.0071 | 0.0000 | 4.57 | 0.84 | 0.79 | 0.91 | 0.57 | 0.88 | 0.93 | 1.00 | 0.78 |
| o_Pseudomonadales | 0.0971 | 0.0862 | 0.4622 | 0.2398 | 0.0000 | 4.76 | 0.89 | 0.88 | 0.98 | 0.70 | 0.87 | 0.90 | 0.95 | 0.78 |

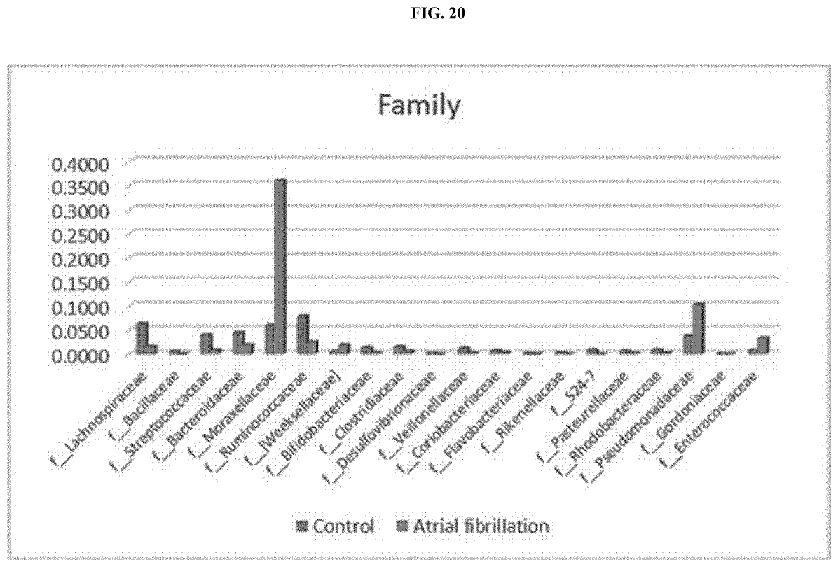

As a result of analyzing bacteria-derived EVs in blood at a family level, a diagnostic model developed using, as a biomarker, one or more bacteria from the family Lachnospiraceae, the family Bacillaceae, the family Streptococcaceae, the family Bacteroidaceae, the family Moraxellaceae, the family Ruminococcaceae, the family Weeksellaceae, the family Bifidobacteriaceae, the family Clostridiaceae, the family Desulfovibrionaceae, the family Veillonellaceae, the family Coriobacteriaceae, the family Flavobacteriaceae, the family Rikenellaceae, the family S24-7, the family Pasteurellaceae, the family Rhodobacteraceae, the family Pseudomonadaceae, the family Gordoniaceae, and the family Enterococcaceae exhibited significant diagnostic performance for atrial fibrillation (see Table 20 and FIG. 20).

TABLE 20

| | | | | | t- | | | | | | | | | |
|---|---|---|---|---|---|---|---|---|---|---|---|---|---|---|
| | | | Atrial | | test | | Training Set | | | | Test Set | | | |
| | Control | | fibrillation | | p- | | | Acu- | Sensi- | Speci- | | Acu- | Sensi- | Speci- |
| Taxon | Mean | SD | Mean | SD | value | Ratio | AUC | rracy | tivity | ficity | AUC | rracy | tivity | ficity |
| f_Desulfovibrionaceae | 0.0011 | 0.0029 | 0.0000 | 0.0000 | 0.0287 | 0.00 | 0.78 | 0.82 | 0.98 | 0.50 | 0.94 | 0.93 | 1.00 | 0.80 |
| f_S24-7 | 0.0086 | 0.0192 | 0.0001 | 0.0003 | 0.0008 | 0.01 | 0.70 | 0.69 | 0.93 | 0.18 | 0.72 | 0.62 | 0.89 | 0.10 |
| f_Bacillaceae | 0.0058 | 0.0072 | 0.0005 | 0.0011 | 0.0000 | 0.09 | 0.86 | 0.79 | 0.76 | 0.86 | 0.75 | 0.66 | 0.68 | 0.60 |
| f_Rikenellaceae | 0.0022 | 0.0059 | 0.0002 | 0.0005 | 0.0114 | 0.10 | 0.71 | 0.64 | 0.84 | 0.23 | 0.55 | 0.62 | 0.89 | 0.10 |
| f_Bifidobacteriaceae | 0.0136 | 0.0154 | 0.0018 | 0.0020 | 0.0000 | 0.13 | 0.80 | 0.76 | 0.80 | 0.68 | 0.73 | 0.62 | 0.74 | 0.40 |
| f_Gordoniaceae | 0.0006 | 0.0019 | 0.0001 | 0.0002 | 0.0357 | 0.13 | 0.54 | 0.69 | 1.00 | 0.05 | 0.37 | 0.55 | 0.84 | 0.00 |
| f_Veillonellaceae | 0.0119 | 0.0201 | 0.0020 | 0.0030 | 0.0003 | 0.17 | 0.77 | 0.70 | 0.84 | 0.41 | 0.80 | 0.59 | 0.79 | 0.20 |
| f_Streptococcaceae | 0.0395 | 0.0638 | 0.0077 | 0.0062 | 0.0002 | 0.19 | 0.85 | 0.82 | 0.93 | 0.59 | 0.81 | 0.83 | 0.89 | 0.70 |
| f_Flavobacteriaceae | 0.0009 | 0.0023 | 0.0002 | 0.0004 | 0.0194 | 0.22 | 0.72 | 0.70 | 0.91 | 0.27 | 0.58 | 0.55 | 0.79 | 0.10 |
| f_Lachnospiraceae | 0.0630 | 0.0694 | 0.0153 | 0.0074 | 0.00 00 | 0.24 | 0.88 | 0.87 | 0.98 | 0.64 | 0.92 | 0.93 | 1.00 | 0.80 |
| f_Ruminococcaceae | 0.0790 | 0.0512 | 0.0247 | 0.0231 | 0.0000 | 0.31 | 0.83 | 0.78 | 0.80 | 0.73 | 0.87 | 0.76 | 0.89 | 0.50 |
| f_Rhodobacteraceae | 0.0081 | 0.0144 | 0.0025 | 0.0045 | 0.0070 | 0.32 | 0.67 | 0.63 | 0.91 | 0.05 | 0.59 | 0.69 | 1.00 | 0.10 |
| f_Pasteurellaceae | 0.0061 | 0.0087 | 0.0020 | 0.0037 | 0.0021 | 0.33 | 0.70 | 0.66 | 0.96 | 0.05 | 0.54 | 0.62 | 0.95 | 0.00 |
| f_Clostridiaceae | 0.0154 | 0.0209 | 0.0060 | 0.0117 | 0.0068 | 0.39 | 0.79 | 0.78 | 0.98 | 0.36 | 0.83 | 0.76 | 1.00 | 0.30 |
| f_Bacteroidaceae | 0.0445 | 0.0348 | 0.0193 | 0.0177 | 0.0000 | 0.43 | 0.85 | 0.82 | 0.84 | 0.77 | 0.85 | 0.76 | 0.84 | 0.60 |
| f_Coriobacteriaceae | 0.0070 | 0.0087 | 0.0031 | 0.0061 | 0.0154 | 0.45 | 0.76 | 0.69 | 0.76 | 0.55 | 0.78 | 0.66 | 0.68 | 0.60 |
| f_Pseudomonadaceae | 0.0377 | 0.0587 | 0.1024 | 0.0473 | 0.0000 | 2.72 | 0.58 | 0.73 | 0.96 | 0.27 | 0.38 | 0.60 | 0.89 | 0.00 |
| f_[Weeksellaceaé] | 0.0058 | 0.0155 | 0.0186 | 0.0123 | 0.0001 | 3.19 | 0.81 | 0.72 | 0.84 | 0.45 | 0.78 | 0.76 | 0.84 | 0.60 |
| f_Enterococcaceae | 0.0076 | 0.0157 | 0.0331 | 0.0113 | 0.0000 | 4.34 | 0.52 | 0.70 | 1.00 | 0.09 | 0.38 | 0.62 | 0.95 | 0.00 |
| f_Moraxellaceae | 0.0594 | 0.0628 | 0.3596 | 0.1972 | 0.0000 | 6.05 | 0.84 | 0.85 | 0.89 | 0.77 | 0.81 | 0.76 | 0.79 | 0.70 |

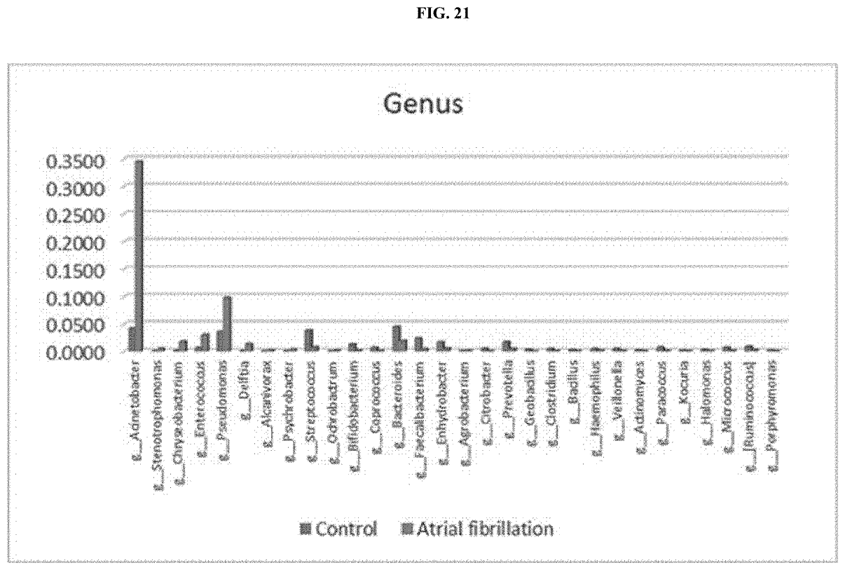

As a result of analyzing bacteria-derived EVs in blood at a genus level, a diagnostic model developed using, as a biomarker, one or more bacteria from the genus Acinetobacter, the genus Stenotrophomonas, the genus Chryseobacterium, the genus Enterococcus, the genus Pseudomonas, the genus Delftia, the genus Alcanivorax, the genus Psychrobacter, the genus Streptococcus, the genus Ochrobactrum, the genus Bifidobacterium, the genus Coprococcus, the genus Bacteroides, the genus Faecalibacterium, the genus Enhydrobacter, the genus Agrobacterium, the genus Citrobacter, the genus Prevotella, the genus Geobacillus, the genus Clostridium, the genus Bacillus, the genus Haemophilus, the genus Veillonella, the genus Actinomyces, the genus Paracoccus, the genus Kocuria, the genus Halomonas, the genus Micrococcus, the genus Ruminococcus, and the genus Porphyromonas exhibited significant diagnostic performance for atrial fibrillation (see Table 21 and FIG. 21).

TABLE 21

| | | | | | t- | | | | | | | | | |
|---|---|---|---|---|---|---|---|---|---|---|---|---|---|---|
| | | | Atrial | | test | | Training Set | | | | Test Set | | | |
| | Control | | fibrillation | | p- | | | Acu- | Sensi- | Speci- | | Acu- | Sensi- | Speci- |
| Taxon | Mean | SD | Mean | SD | value | Ratio | AUC | rracy | tivity | ficity | AUC | rracy | tivity | ficity |
| g_Geobacillus | 0.0036 | 0.0066 | 0.0000 | 0.0000 | 0.0132 | 0.00 | 0.73 | 0.66 | 0.79 | 0.42 | 0.71 | 0.69 | 0.86 | 0.25 |
| g_Citrobacter | 0.0048 | 0.0118 | 0.0003 | 0.0008 | 0.0034 | 0.06 | 0.75 | 0.69 | 0.81 | 0.46 | 0.45 | 0.48 | 0.62 | 0.13 |
| g_Coprococcus | 0.0067 | 0.0075 | 0.0008 | 0.0011 | 0.0000 | 0.12 | 0.78 | 0.64 | 0.67 | 0.58 | 0.80 | 0.69 | 0.71 | 0.63 |
| g_Bifidobacterium | 0.0130 | 0.0154 | 0.0016 | 0.0020 | 0.0000 | 0.13 | 0.84 | 0.76 | 0.87 | 0.50 | 0.66 | 0.55 | 0.71 | 0.33 |
| g_Faecalibacterium | 0.0239 | 0.0286 | 0.0040 | 0.0058 | 0.0000 | 0.17 | 0.76 | 0.75 | 0.89 | 0.40 | 0.80 | 0.62 | 0.94 | 0.17 |
| g_Bacillus | 0.0022 | 0.0033 | 0.0004 | 0.0011 | 0.0002 | 0.19 | 0.70 | 0.67 | 0.84 | 0.38 | 0.51 | 0.69 | 0.86 | 0.25 |
| g_Streptococcus | 0.0381 | 0.0638 | 0.0076 | 0.0062 | 0.0004 | 0.20 | 0.87 | 0.79 | 0.87 | 0.60 | 0.73 | 0.69 | 0.65 | 0.75 |
| g_Clostridium | 0.0043 | 0.0103 | 0.0009 | 0.0032 | 0.0173 | 0.20 | 0.71 | 0.67 | 0.77 | 0.50 | 0.40 | 0.55 | 0.71 | 0.13 |
| g_Veillonella | 0.0046 | 0.0067 | 0.0010 | 0.0021 | 0.0002 | 0.23 | 0.69 | 0.66 | 0.88 | 0.25 | 0.64 | 0.72 | 0.95 | 0.13 |
| g_Paracoccus | 0.0074 | 0.0144 | 0.0017 | 0.0043 | 0.0052 | 0.23 | 0.69 | 0.63 | 0.83 | 0.15 | 0.57 | 0.52 | 0.88 | 0.00 |
| g_Porphyromonas | 0.0017 | 0.0033 | 0.0004 | 0.0010 | 0.0061 | 0.25 | 0.64 | 0.67 | 0.98 | 0.13 | 0.49 | 0.69 | 0.95 | 0.61 |
| g_Kocuria | 0.0014 | 0.0022 | 0.0003 | 0.0005 | 0.0005 | 0.25 | 0.68 | 0.66 | 0.86 | 0.29 | 0.42 | 0.66 | 0.81 | 0.25 |
| g_Micrococcus | 0.0066 | 0.0113 | 0.0017 | 0.0016 | 0.0013 | 0.26 | 0.67 | 0.70 | 0.98 | 0.05 | 0.62 | 0.55 | 0.94 | 0.00 |
| g_Halomonas | 0.0026 | 0.0036 | 0.0007 | 0.0021 | 0.0018 | 0.26 | 0.67 | 0.67 | 0.98 | 0.13 | 0.57 | 0.69 | 0.95 | 0.00 |
| g_Prevotella | 0.0171 | 0.0240 | 0.0047 | 0.0059 | 0.0002 | 0.27 | 0.74 | 0.70 | 0.89 | 0.25 | 0.64 | 0.59 | 0.88 | 0.17 |
| g_[Ruminococcus] | 0.0087 | 0.0219 | 0.0028 | 0.0032 | 0.0420 | 0.32 | 0.65 | 0.72 | 0.98 | 0.10 | 0.52 | 0.55 | 0.94 | 0.00 |
| g_Enhydrobacter | 0.0164 | 0.0217 | 0.0054 | 0.0058 | 0.0003 | 0.33 | 0.76 | 0.66 | 0.78 | 0.35 | 0.47 | 0.41 | 0.71 | 0.00 |
| g_Actinomyces | 0.0019 | 0.0025 | 0.0006 | 0.0008 | 0.0004 | 0.33 | 0.69 | 0.67 | 0.86 | 0.33 | 0.62 | 0.66 | 0.81 | 0.25 |
| g_Haemophilus | 0.0045 | 0.0056 | 0.0018 | 0.0036 | 0.0062 | 0.40 | 0.70 | 0.61 | 0.88 | 0.13 | 0.51 | 0.69 | 0.90 | 0.13 |
| g_Bacteroides | 0.0445 | 0.0348 | 0.0193 | 0.0177 | 0.0000 | 0.43 | 0.78 | 0.72 | 0.87 | 0.35 | 0.68 | 0.59 | 0.88 | 0.17 |
| g_Agrobacterium | 0.0005 | 0.0016 | 0.0014 | 0.0018 | 0.0166 | 2.74 | 0.75 | 0.70 | 0.91 | 0.33 | 0.37 | 0.66 | 0.90 | 0.00 |
| g_Pseudomonas | 0.0356 | 0.0578 | 0.0980 | 0.0463 | 0.0000 | 2.75 | 0.91 | 0.88 | 0.94 | 0.75 | 0.70 | 0.72 | 0.88 | 0.50 |
| g_Enterococcus | 0.0067 | 0.0143 | 0.0302 | 0.0114 | 0.0000 | 4.49 | 0.92 | 0.85 | 0.94 | 0.65 | 1.00 | 0.76 | 1.00 | 0.24 |
| g_Acinetobacter | 0.0419 | 0.0569 | 0.3448 | 0.1938 | 0.0000 | 8.22 | 0.95 | 0.91 | 0.98 | 0.75 | 0.74 | 0.83 | 1.00 | 0.58 |
| g_Alcanivorax | 0.0002 | 0.0011 | 0.0019 | 0.0019 | 0.0000 | 9.44 | 0.89 | 0.81 | 0.98 | 0.50 | 0.74 | 0.79 | 0.95 | 0.38 |
| g_Chryseobacterium | 0.0017 | 0.0032 | 0.0181 | 0.0126 | 0.0000 | 10.8 | 0.93 | 0.90 | 0.96 | 0.75 | 0.73 | 0.83 | 1.00 | 0.58 |
| g_Psychrobacter | 0.0003 | 0.0009 | 0.0033 | 0.0025 | 0.0000 | 10.88 | 0.88 | 0.85 | 0.93 | 0.71 | 0.69 | 0.86 | 1.00 | 0.50 |
| g_Stenotrophomonas | 0.0002 | 0.0008 | 0.0049 | 0.0040 | 0.0000 | 20.54 | 0.94 | 0.90 | 0.95 | 0.79 | 0.70 | 0.83 | 0.90 | 0.63 |
| g_Ochrobactrum | 0.0001 | 0.0004 | 0.0017 | 0.0019 | 0.0000 | 23.19 | 0.87 | 0.82 | 0.95 | 0.58 | 0.73 | 0.90 | 1.00 | 0.63 |
| g_Delftia | 0.0003 | 0.0013 | 0.0135 | 0.0116 | 0.0000 | 42.36 | 0.91 | 0.91 | 0.98 | 0.75 | 0.76 | 0.83 | 0.94 | 0.67 |

The above description of the present invention is provided only for illustrative purposes, and it will be understood by one of ordinary skill in the art to which the present invention pertains that the invention may be embodied in various modified forms without departing from the spirit or essential characteristics thereof. Thus, the embodiments described herein should be considered in an illustrative sense only and not for the purpose of limitation.

### INDUSTRIAL APPLICABILITY

According to the present invention, a risk group for heart disease can be diagnosed early and predicted by diagnosing a causative factor of heart disease through metagenomic analysis of bacteria-derived extracellular vesicles from a human body-derived sample, and thus the onset of heart disease can be delayed or heart disease may be prevented through appropriate management, and, even after heart disease occurs, early diagnosis for heart disease can be implemented, thereby lowering a disease rate and increasing therapeutic effects. In addition, patients diagnosed with heart disease are able to avoid exposure to causative factors predicted by metagenomic analysis, whereby the progression of heart disease is ameliorated, or recurrence of heart disease can be prevented.

1. A method of providing information for heart disease diagnosis, the method comprising:
(a) extracting DNA from extracellular vesicles isolated from a subject sample;
(b) performing polymerase chain reaction (PCR) on the extracted DNA using a pair of primers having SEQ ID NO:1 and SEQ ID NO: 2; and
(c) comparing an increase or decrease in content of bacteria-derived extracellular vesicles of the subject sample with that of a normal individual-derived sample through sequencing of a product of the PCR,
wherein the comparing comprises comparing an increase or decrease in content of:
extracellular vesicles derived from one or more bacteria selected from the group consisting of the phylum Acidobacteria, the phylum Firmicutes, the phylum Crenarchaeota, the phylum Planctomycetes, the phylum Chloroflexi, the phylum Euryarchaeota, the phylum WS3, the phylum Nitrospirae, the phylum WPS-2, the phylum AD3, the phylum Verrucomicrobia, the phylum Gemmatimonadetes, the phylum Proteobacteria, the phylum TM7, and the phylum Cyanobacteria;
extracellular vesicles derived from one or more bacteria selected from the group consisting of the class Acidobacteriia, the class DA052, the class Methanomicrobia, the class Thaumarchaeota, the class Clostridia, the class Coriobacteriia, the class Betaproteobacteria, the class Ktedonobacteria, the class Planctomycetia, the class Solibacteres, the class Erysipelotrichi, the class Verrucomicrobiae, the class TM7-3, the class Bacteroidia, the class Phycisphaerae, the class MCG the class Nitrospira, the class Pedosphaerae, the class Thermoleophilia, the class Saprospirae, the class PRR-12, the class Spartobacteria, the class Acidimicrobiia, the class TM1, the class Deltaproteobacteria, the class Anaerolineae, the class Thermoplasmata, the class Chthonomonadetes, the class Acidobacteria-6, the class Fusobacteriia, the class Fimbriimonadia, the class Actinobacteria, the class Flavobacteriia, and the class Chloroplast;
extracellular vesicles derived from one or more bacteria selected from the group consisting of the order Lactobacillales, the order Acidobacteriales, the order Enterobacteriales, the order Xanthomonadales, the order Clostridiales, the order Coriobacteriales, the order Ellin6513, the order Burkholderiales, the order Erysipelotrichales, the order Solibacterales, the order Verrucomicrobiales, the order Rhodospirillales, the order Gemmatales, the order Thermogemmatisporales, the order Saprospirales, the order Acidimicrobiales, the order Pedosphaerales, the order Bifidobacteriales, the order Chthoniobacterales, the order Solirubrobacterales, the order Syntrophobacterales, the order Bacteroidales, the order Nitrospirales, the order Ktedonobacterales, the order WD2101, the order iiil-15, the order Ellin329, the order Thiotrichales, the order Myxococcales, the order Stramenopiles, the order Vibrionales, the order Pseudomonadales, the order Bacillales, the order Sphingomonadales, the order Turicibacterales, the order Fimbriimonadales, the order Deinococcales, the order Pasteurellales, the order Flavobacteriales, the order Actinomycetales, the order Rhodobacterales, and the order Streptophyta;
extracellular vesicles derived from one or more bacteria selected from the group consisting of the family Koribacteraceae, the family Comamonadaceae, the family Enterobacteriaceae, the family Streptococcaceae, the family Coriobacteriaceae, the family Lachnospiraceae, the family Prevotellaceae, the family Ruminococcaceae, the family Xanthomonadaceae, the family Propionibacteriaceae, the family Hyphomicrobiaceae, the family Verrucomicrobiaceae, the family Solibacteraceae, the family Acidobacteriaceae, the family Erysipelotrichaceae, the family Ktedonobacteraceae, the family Thermogemmatisporaceae, the family Moraxellaceae, the family Veillonellaceae, the family Burkholderiaceae, the family Rhodospirillaceae, the family Bifidobacteriaceae, the family Gemmataceae, the family Streptomycetaceae, the family Chitinophagaceae, the family Brucellaceae, the family Rhizobiaceae, the family Chthoniobacteraceae, the family Sinobacteraceae, the family Conexibacteraceae, the family Oxalobacteraceae, the family Isosphaeraceae, the family Ellin515, the family Piscirickettsiaceae, the family Methylocystaceae, the family Pseudomonadaceae, the family Clostridiaceae, the family Sphingomonadaceae, the family Turicibacteraceae, the family Staphylococcaceae, the family Deinococcaceae, the family Methylobacteriaceae, the family Aerococcaceae, the family Fusobacteriaceae, the family Fimbriimonadaceae, the family Bacillaceae, the family Planococcaceae, the family Bacteroidaceae, the family Weeksellaceae, the family Desulfovibrionaceae, the family Flavobacteriaceae, the family Rikenellaceae, the family S24-7, the family Pasteurellaceae, the family Rhodobacteraceae, the family Gordoniaceae, and the family Enterococcaceae; or
extracellular vesicles derived from one or more bacteria selected from the group consisting of the genus Delftia, the genus Agrobacterium, the genus Stenotrophomonas, the genus Faecalibacterium, the genus Candidatus Koribacter, the genus Akkermansia, the genus Streptococcus, the genus Salinispora, the genus Candidatus Solibacter, the genus Citrobacter, the genus Collinsella, the genus Burkholderia, the genus Coprococcus, the genus Rhodoplanes, the genus Acinetobacter, the genus Prevotella, the genus Propionibacterium, the genus Lactococcus, the genus Bifidobacterium, the genus Methanobacterium, the genus Micrococcus, the genus Methanocella, the genus Brevibacterium, the genus Streptacidiphilus, the genus Streptomyces, the genus Ochrobactrum, the genus Methanosaeta, the genus Lysinibacillus, the genus Cupriavidus, the genus Pseudomonas, the genus Clostridium, the genus Sphingomonas, the genus Staphylococcus, the genus Thermoanaerobacterium, the genus Neisseria, the genus Enhydrobacter, the genus Actinomyces, the genus Turicibacter, the genus Phascolarctobacterium, the genus Catenibacterium, the genus Bacillus, the genus Deinococcus, the genus Fusobacterium, the genus Adlercreutzia, the genus Chryseobacterium, the genus Enterococcus, the genus Alcanivorax, the genus Psychrobacter, the genus Bacteroides, the genus Geobacillus, the genus Haemophilus, the genus Veillonella, the genus Paracoccus, the genus Kocuria, the genus Halomonas, the genus Ruminococcus, and the genus Porphyromonas.

2. The method of claim 1, wherein the heart disease is myocardial infarction, cardiomyopathy, variant angina, or atrial fibrillation.

3. The method of claim 2, wherein, in process (c), myocardial infarction is diagnosed by at least one selected from the group consisting of: comparing an increase or decrease in content of extracellular vesicles derived from one or more bacteria selected from the group consisting of the phylum Acidobacteria, the phylum Firmicutes, the phylum Crenarchaeota, the phylum Planctomycetes, the phylum Chloroflexi, the phylum Euryarchaeota, the phylum WS3, the phylum Nitrospirae, the phylum WPS-2, and the phylum AD3;
comparing an increase or decrease in content of extracellular vesicles derived from one or more bacteria selected from the group consisting of the class Acidobacteriia, the class DA052, the class Methanomicrobia, the class Thaumarchaeota, the class Clostridia, the class Coriobacteriia, the class Betaproteobacteria, the class Ktedonobacteria, the class Planctomycetia, the class Solibacteres, the class Erysipelotrichi, the class Verrucomicrobiae, the class TM7-3, the class Bacteroidia, the class Phycisphaerae, the class MCU the class Nitrospira, the class Pedosphaerae, the class Thermoleophilia, the class Saprospirae, the class PRR-12, the class Spartobacteria, the class Acidimicrobiia, the class TM1, the class Deltaproteobacteria, the class Anaerolineae, the class Thermoplasmata, the class Chthonomonadetes, and the class Acidobacteria-6;
comparing an increase or decrease in content of extracellular vesicles derived from one or more bacteria selected from the group consisting of the order Lactobacillales, the order Acidobacteriales, the order Enterobacteriales, the order Xanthomonadales, the order Clostridiales, the order Coriobacteriales, the order Ellin6513, the order Burkholderiales, the order Erysipelotrichales, the order Solibacterales, the order Verrucomicrobiales, the order Rhodospirillales, the order Gemmatales, the order Thermogemmatisporales, the order Saprospirales, the order Acidimicrobiales, the order Pedosphaerales, the order Bifidobacteriales, the order Chthoniobacterales, the order Solirubrobacterales, the order Syntrophobacterales, the order Bacteroidales, the order Nitrospirales, the order Ktedonobacterales, the order WD2101, the order iiil-15, the order Ellin329, the order Thiotrichales, the order Myxococcales, the order Stramenopiles, and the order Vibrionales;
comparing an increase or decrease in content of extracellular vesicles derived from one or more bacteria selected from the group consisting of the family Koribacteraceae, the family Comamonadaceae, the family Enterobacteriaceae, the family Streptococcaceae, the family Coriobacteriaceae, the family Lachnospiraceae, the family Prevotellaceae, the family Ruminococcaceae, the family Xanthomonadaceae, the family Propionibacteriaceae, the family Hyphomicrobiaceae, the family Verrucomicrobiaceae, the family Solibacteraceae, the family Acidobacteriaceae, the family Erysipelotrichaceae, the family Ktedonobacteraceae, the family Thermogemmatisporaceae, the family Moraxellaceae, the family Veillonellaceae, the family Burkholderiaceae, the family Rhodospirillaceae, the family Bdobacteriaceae, the family Gemmataceae, the family Streptomycetaceae, the family Chitinophagaceae, the family Brucellaceae, the family Rhizobiaceae, the family Chthoniobacteraceae, the family Sinobacteraceae, the family Conexibacteraceae, the family Oxalobacteraceae, the family Isosphaeraceae, the family Ellin515, the family Piscirickettsiaceae, and the family Methylocystaceae; and
comparing an increase or decrease in content of extracellular vesicles derived from one or more bacteria selected from the group consisting of the genus Delftia, the genus Agrobacterium, the genus Stenotrophomonas, the genus Faecalibacterium, the genus Candidatus Koribacter, the genus Akkermansia, the genus Streptococcus, the genus Salinispora, the genus Candidatus Solibacter, the genus Citrobacter, the genus Collinsella, the genus Burkholderia, the genus Coprococcus, the genus Rhodoplanes, the genus Acinetobacter, the genus Prevotella, the genus Propionibacterium, the genus Lactococcus, the genus Bifidobacterium, the genus Methanobacterium, the genus Micrococcus, the genus Methanocella, the genus Brevibacterium, the genus Streptacidiphilus, the genus Streptomyces, the genus Ochrobactrum, the genus Methanosaeta, the genus Lysinibacillus, and the genus Cupriavidus.

4.-7. (canceled)

8. The method of claim 2, wherein, in process (c), cardiomyopathy is diagnosed by at least one selected from the group consisting of:
comparing an increase or decrease in content of extracellular vesicles derived from one or more bacteria selected from the group consisting of the phylum Verrucomicrobia, the phylum Acidobacteria, the phylum Gemmatimonadetes, and the phylum Planctomycetes;
comparing an increase or decrease in content of extracellular vesicles derived from one or more bacteria selected from the group consisting of the class Verrucomicrobiae, the class Fusobacteriia, the class Acidobacteriia, the class Planctomycetia, the class DA052, the class Deltaproteobacteria, and the class Acidimicrobiia;
comparing an increase or decrease in content of extracellular vesicles derived from one or more bacteria selected from the group consisting of the order Pseudomonadales, the order Bacillales, the order Acidobacteriales, the order Sphingomonadales, the order Verrucomicrobiales, the order Turicibacterales, the order Acidimicrobiales, the order Ellin6513, the order Xanthomonadales, and the order Gemmatales;
comparing an increase or decrease in content of extracellular vesicles derived from one or more bacteria selected from the group consisting of the family Pseudomonadaceae, the family Clostridiaceae, the family Comamonadaceae, the family Oxalobacteraceae, the family Moraxellaceae, the family Verrucomicrobiaceae, the family Koribacteraceae, the family Sphingomonadaceae, the family Turicibacteraceae, the family Xanthomonadaceae, the family Gemmataceae, and the family Staphylococcaceae; and
comparing an increase or decrease in content of extracellular vesicles derived from one or more bacteria selected from the group consisting of the genus Pseudomonas, the genus Clostridium, the genus Cupriavidus, the genus Acinetobacter, the genus Citrobacter, the genus Sphingomonas, the genus Candidatus Koribacter, the genus Staphylococcus, the genus Thermoanaerobacterium, the genus Micrococcus, the genus Akkermansia, the genus Neisseria, the genus Enhydrobacter, the genus Actinomyces, the genus Turicibacter, the genus Phascolarctobacterium, the genus Lactococcus, the genus Delftia, and the genus Stenotrophomonas.

9.-12. (canceled)

13. The method of claim 2, wherein, in process (c), variant angina is diagnosed by at least one selected from the group consisting of:
comparing an increase or decrease in content of extracellular vesicles derived from one or more bacteria selected from the group consisting of the phylum Verrucomicrobia, the phylum Acidobacteria, the phylum Planctomycetes, the phylum Gemmatimonadetes, the phylum Chloroflexi, and the phylum Euryarchaeota;
comparing an increase or decrease in content of extracellular vesicles derived from one or more bacteria selected from the group consisting of the class Verrucomicrobiae, the class Acidobacteriia, the class Fimbriimonadia, the class Erysipelotrichi, the class Ktedonobacteria, and the class Deltaproteobacteria;
comparing an increase or decrease in content of extracellular vesicles derived from one or more bacteria selected from the group consisting of the order Pseudomonadales, the order Erysipelotrichales, the order Fimbriimonadales, the order Acidobacteriales, the order Verrucomicrobiales, the order Xanthomonadales, the order Myxococcales, the order Deinococcales, and the order Rhodospirillales;
comparing an increase or decrease in content of extracellular vesicles derived from one or more bacteria selected from the group consisting of the family Koribacteraceae, the family Oxalobacteraceae, the family Comamonadaceae, the family Moraxellaceae, the family Pseudomonadaceae, the family Hyphomicrobiaceae, the family Erysipelotrichaceae, the family Deinococcaceae, the family Clostridiaceae, the family Verrucomicrobiaceae, the family Sinobacteraceae, the family Rhodospirillaceae, the family Methylobacteriaceae, the family Aerococcaceae, the family Fusobacteriaceae, the family Fimbriimonadaceae, the family Bacillaceae, and the family Planococcaceae; and
comparing an increase or decrease in content of extracellular vesicles derived from one or more bacteria selected from the group consisting of the genus Citrobacter, the genus Acinetobacter, the genus Cupriavidus, the genus Clostridium, the genus Catenibacterium, the genus Pseudomonas, the genus Lactococcus, the genus Stenotrophomonas, the genus Akkermansia, the genus Bacillus, the genus Delftia, the genus Agrobacterium, the genus Deinococcus, the genus Fusobacterium, and the genus Adlercreutzia.

14.-17. (canceled)

18. The method of claim 2, wherein, in process (c), atrial fibrillation is diagnosed by at least one selected from the group consisting of:
comparing an increase or decrease in content of extracellular vesicles derived from one or more bacteria selected from the group consisting of the phylum Proteobacteria, the phylum TM7, the phylum Chloroflexi, the phylum Acidobacteria, and the phylum Cyanobacteria;
comparing an increase or decrease in content of extracellular vesicles derived from one or more bacteria selected from the group consisting of the class Clostridia, the class Bacteroidia, the class Actinobacteria, the class Flavobacteriia, the class Erysipelotrichi, the class TM7-3, and the class Chloroplast;
comparing an increase or decrease in content of extracellular vesicles derived from one or more bacteria selected from the group consisting of the order Pseudomonadales, the order Clostridiales, the order Bacteroidales, the order Enterobacteriales, the order Xanthomonadales, the order Bifidobacteriales, the order Pasteurellales, the order Flavobacteriales, the order Actinomycetales, the order Rhodobacterales, the order Coriobacteriales, the order Erysipelotrichales, and the order Streptophyta;
comparing an increase or decrease in content of extracellular vesicles derived from one or more bacteria selected from the group consisting of the family Lachnospiraceae, the family Bacillaceae, the family Streptococcaceae, the family Bacteroidaceae, the family Moraxellaceae, the family Ruminococcaceae, the family Weeksellaceae, the family Bifidobacteriaceae, the family Clostridiaceae, the family Desulfovibrionaceae, the family Veillonellaceae, the family Coriobacteriaceae, the family Flavobacteriaceae, the family Rikenellaceae, the family S24-7, the family Pasteurellaceae, the family Rhodobacteraceae, the family Pseudomonadaceae, the family Gordoniaceae, and the family Enterococcaceae; and
comparing an increase or decrease in content of extracellular vesicles derived from one or more bacteria selected from the group consisting of the genus Acinetobacter, the genus Stenotrophomonas, the genus Chryseobacterium, the genus Enterococcus, the genus Pseudomonas, the genus Delftia, the genus Alcanivorax, the genus Psychrobacter, the genus Streptococcus, the genus Ochrobactrum, the genus Bifidobacterium, the genus Coprococcus, the genus Bacteroides, the genus Faecalibacterium, the genus Enhydrobacter, the genus Agrobacterium, the genus Citrobacter, the genus Prevotella, the genus Geobacillus, the genus Clostridium, the genus Bacillus, the genus Haemophilus, the genus Veillonella, the genus Actinomyces, the genus Paracoccus, the genus Kocuria, the genus Halomonas, the genus Micrococcus, the genus Ruminococcus, and the genus Porphyromonas.

19.-22. (canceled)

23. The method of claim 1, wherein the subject sample is blood.

24. The method of claim 23, wherein the blood is whole blood, serum, plasma, or blood mononuclear cells.

25. A method of diagnosing heart disease, the method comprising:
(a) extracting DNA from extracellular vesicles isolated from a subject sample;
(b) performing polymerase chain reaction (PCR) on the extracted DNA using a pair of primers having SEQ ID NO:1 and SEQ ID NO: 2; and
(c) comparing an increase or decrease in content of bacteria-derived extracellular vesicles of the subject sample with that of a normal individual-derived sample through sequencing of a product of the PCR,
wherein the comparing comprises comparing an increase or decrease in content of:
extracellular vesicles derived from one or more bacteria selected from the group consisting of the phylum Acidobacteria, the phylum Firmicutes, the phylum Crenarchaeota, the phylum Planctomycetes, the phylum Chloroflexi, the phylum Euryarchaeota, the phylum WS3, the phylum Nitrospirae, the phylum WPS-2, the phylum AD3, the phylum Verrucomicrobia, the phylum Gemmatimonadetes, the phylum Proteobacteria, the phylum TM7, and the phylum Cyanobacteria;
extracellular vesicles derived from one or more bacteria selected from the group consisting of the class Acidobacteriia, the class DA052, the class Methanomicrobia, the class Thaumarchaeota, the class Clostridia, the class Coriobacteriia, the class Betaproteobacteria, the class Ktedonobacteria, the class Planctomycetia, the class Solibacteres, the class Erysipelotrichi, the class Verrucomicrobiae, the class TM7-3, the class Bacteroidia, the class Phycisphaerae, the class MCG the class Nitrospira, the class Pedosphaerae, the class Thermoleophilia, the class Saprospirae, the class PRR-12, the class Spartobacteria, the class Acidimicrobiia, the class TM1, the class Deltaproteobacteria, the class Anaerolineae, the class Thermoplasmata, the class Chthonomonadetes, the class Acidobacteria-6, the class Fusobacteriia, the class Fimbriimonadia, the class Actinobacteria, the class Flavobacteriia, and the class Chloroplast;
extracellular vesicles derived from one or more bacteria selected from the group consisting of the order Lactobacillales, the order Acidobacteriales, the order Enterobacteriales, the order Xanthomonadales, the order Clostridiales, the order Coriobacteriales, the order Ellin6513, the order Burkholderiales, the order Erysipelotrichales, the order Solibacterales, the order Verrucomicrobiales, the order Rhodospirillales, the order Gemmatales, the order Thermogemmatisporales, the order Saprospirales, the order Acidimicrobiales, the order Pedosphaerales, the order Bifidobacteriales, the order Chthoniobacterales, the order Solirubrobacterales, the order Syntrophobacterales, the order Bacteroidales, the order Nitrospirales, the order Ktedonobacterales, the order WD2101, the order iiil-15, the order Ellin329, the order Thiotrichales, the order Myxococcales, the order Stramenopiles, the order Vibrionales, the order Pseudomonadales, the order Bacillales, the order Sphingomonadales, the order Turicibacterales, the order Fimbriimonadales, the order Deinococcales, the order Pasteurellales, the order Flavobacteriales, the order Actinomycetales, the order Rhodobacterales, and the order Streptophyta;
extracellular vesicles derived from one or more bacteria selected from the group consisting of the family Koribacteraceae, the family Comamonadaceae, the family Enterobacteriaceae, the family Streptococcaceae, the family Coriobacteriaceae, the family Lachnospiraceae, the family Prevotellaceae, the family Ruminococcaceae, the family Xanthomonadaceae, the family Propionibacteriaceae, the family Hyphomicrobiaceae, the family Verrucomicrobiaceae, the family Solibacteraceae, the family Acidobacteriaceae, the family Erysipelotrichaceae, the family Ktedonobacteraceae, the family Thermogemmatisporaceae, the family Moraxellaceae, the family Veillonellaceae, the family Burkholderiaceae, the family Rhodospirillaceae, the family Bifidobacteriaceae, the family Gemmataceae, the family Streptomycetaceae, the family Chitinophagaceae, the family Brucellaceae, the family Rhizobiaceae, the family Chthoniobacteraceae, the family Sinobacteraceae, the family Conexibacteraceae, the family Oxalobacteraceae, the family Isosphaeraceae, the family Ellin515, the family Piscirickettsiaceae, the family Methylocystaceae, the family Pseudomonadaceae, the family Clostridiaceae, the family Sphingomonadaceae, the family Turicibacteraceae, the family Staphylococcaceae, the family Deinococcaceae, the family Methylobacteriaceae, the family Aerococcaceae, the family Fusobacteriaceae, the family Fimbriimonadaceae, the family Bacillaceae, the family Planococcaceae, the family Bacteroidaceae, the family Weeksellaceae, the family Desulfovibrionaceae, the family Flavobacteriaceae, the family Rikenellaceae, the family S24-7, the family Pasteurellaceae, the family Rhodobacteraceae, the family Gordoniaceae, and the family Enterococcaceae; or
extracellular vesicles derived from one or more bacteria selected from the group consisting of the genus Delftia, the genus Agrobacterium, the genus Stenotrophomonas, the genus Faecalibacterium, the genus Candidatus Koribacter, the genus Akkermansia, the genus Streptococcus, the genus Salinispora, the genus Candidatus Solibacter, the genus Citrobacter, the genus Collinsella, the genus Burkholderia, the genus Coprococcus, the genus Rhodoplanes, the genus Acinetobacter, the genus Prevotella, the genus Propionibacterium, the genus Lactococcus, the genus Bifidobacterium, the genus Methanobacterium, the genus Micrococcus, the genus Methanocella, the genus Brevibacterium, the genus Streptacidiphilus, the genus Streptomyces, the genus Ochrobactrum, the genus Methanosaeta, the genus Lysinibacillus, the genus Cupriavidus, the genus Pseudomonas, the genus Clostridium, the genus Sphingomonas, the genus Staphylococcus, the genus Thermoanaerobacterium, the genus Neisseria, the genus Enhydrobacter, the genus Actinomyces, the genus Turicibacter, the genus Phascolarctobacterium, the genus Catenibacterium, the genus Bacillus, the genus Deinococcus, the genus Fusobacterium, the genus Adlercreutzia, the genus Chryseobacterium, the genus Enterococcus, the genus Alcanivorax, the genus Psychrobacter, the genus Bacteroides, the genus Geobacillus, the genus Haemophilus, the genus Veillonella, the genus Paracoccus, the genus Kocuria, the genus Halomonas, the genus Ruminococcus, and the genus Porphyromonas.


SEQUENCE LISTING (2 sequences)

SEQ ID NO: 1 - DNA, 50 bp, Artificial Sequence
  16S_V3_F
  misc_feature (42)..(42): any nucleotide
tcgtcggcagcgtcagatgtgtataagagacagcctacgggnggcwgcag

SEQ ID NO: 2 - DNA, 55 bp, Artificial Sequence
  16S_V4_R
gtctcgtgggctcggagatgtgtataagagacaggactachvgggtatctaatcc